(19) United States
(12) Patent Application Publication
(10) Pub. No.: US 2025/0099534 A1
(43) Pub. Date: Mar. 27, 2025

(54) UBIQUITIN HIGH AFFINITY CYCLIC PEPTIDES AND METHODS OF USE THEREOF

(71) Applicants: TECHNION RESEARCH & DEVELOPMENT FOUNDATION LIMITED, Haifa (IL); THE UNIVERSITY OF TOKYO, Tokyo (JP)

(72) Inventors: Ashraf BRIK, Haifa (IL); Guy KAMNESKY, Haifa (IL); Hiroaki SUGA, Tokyo (JP); Yichao HUANG, Tokyo (JP); Joseph ROGERS, Tokyo (JP)

(21) Appl. No.: 18/969,949

(22) Filed: Dec. 5, 2024

Related U.S. Application Data
(63) Continuation of application No. PCT/IL2023/050583, filed on Jun. 6, 2023.
(60) Provisional application No. 63/349,366, filed on Jun. 6, 2022.

(51) Int. Cl. A61K 38/12; C07K 7/64

(52) U.S. Cl. CPC A61K 38/12; C07K 7/64

(57) ABSTRACT

The present invention provides cyclic peptides, as well as methods of using the same, such as for ameliorating or treating a K63Ub-related disease in a subject in need thereof.

### CROSS-REFERENCE TO RELATED APPLICATIONS

This application is a Bypass Continuation of PCT Patent Application No. PCT/IL2023/050583 having International filing date of Jun. 6, 2023, which claims the benefit of priority of U.S. Provisional Application No. 63/349,366, titled “UBIQUITIN HIGH AFFINITY CYCLIC PEPTIDES AND METHODS OF USE THEREOF”, filed 6 Jun. 2022, the contents of which are incorporated herein by reference in their entirety.

### REFERENCE TO AN ELECTRONIC SEQUENCE LISTING

The contents of the electronic sequence listing (TECH-TOK-P-0269-PCT.xml; size: 15,910 bytes; and date of creation: Jun. 5, 2023) is herein incorporated by reference in its entirety.

### FIELD OF INVENTION

The present invention is in the field of peptide engineering and drug screening.

### BACKGROUND OF THE INVENTION

Ubiquitination is a complex post-translational modification (PTM) and is involved in various cellular processes. In ubiquitination, the C-terminal glycine of ubiquitin (Ub) is attached mainly to the ε-amine side chain of a lysine residue of a substrate protein—a process that is achieved by the action of three enzymes known as the E1-E3. PolyUb chains with different linkages (e.g., Lys63-linked Ub chains) can be formed by elongation of Ub via the addition of another Ub, to one of its seven lysine residues (e.g., Lys63) or N-terminus. Importantly, Ub chains with the different linkage types have distinct topologies and dynamics, where each Ub chain is recognized by a specific subset of cellular proteins. As a result, each chain could lead to a particular cell signaling, such as proteasomal degradation (e.g., Lys48-linked Ub chains), mitophagy, cell-cycle regulation, protein trafficking, autophagy, DNA repair (e.g., Lys63-linked Ub chain), and immune response. Like most other PTMs, ubiquitination is a reversible process in which a family of enzymes known as deubiquitinases (DUBs) trims or completely detaches the Ub chain from the ubiquitinated protein.

The major components of the Ub system (e.g., DUBs, E1-E3s, and 26S proteasome) are well-known targets in drug development, in which some of these already resulted in approved cancer drugs (e.g., Bortezomib). While most approaches focus on interfering with the activity of a specific enzyme involved in the Ub system, a different approach has emerged to target the Ub chain itself, as the code of signaling. Recently, we discovered a class of cyclic peptides, which bind specifically to Lys48-linked Ub chains, leading to interference with the specific DUBs as well as proteasomal degradation of ubiquitinated proteins. These cyclic peptides, which were selected using the Random Non-standard Peptides Integrated Discovery (RaPID) approach against synthetic Lys48-linked Ub chains, exhibited a high level of apoptosis and attenuated tumor growth in vivo.

Having succeeded with Lys48-linked chains using cyclic peptide modulators, we aimed to push the boundaries of this approach and explore targeting the Lys63-linked chains. These chains are known to be the second most predominant class after Lys48-linked Ub chains in cells. Finding a selective modulator for the Lys63-linked chains should allow us to interfere with other biological pathways, as they are involved in non-proteolytic cellular processes, such as DNA damage repair (DDR). A major challenge with this approach stems from the structural features of the Lys63-linked Ub chain, which has been shown to adopt an opened structure in the crystal and an ensemble of conformations in solution. This is vastly different from the more defined and closed conformation of the Lys48-linked Ub chains. Moreover, in Lys63-linked Ub chains, the hydrophobic patches of the proximal Ub monomer and the distal Ub do not lie on the same surface, presumably making each Ub in this chain behave as a separate unit. Therefore, developing specific cyclic peptide modulators that could interact with both Ub units and interfere with the function of these chains is very challenging and remained an unexplored area.

The RaPID method has gained special attention due to its ability to generate diverse libraries, with a unique chemical space, each composed of up to trillion thioether-macrocyclic peptides, using in vitro translation against a protein of interest (POI). Combining this feature with the inventors ability to synthesize any of the Ub chains with a defined length, linkage, and high purity, should allow us to selectively target any of the desired chains.

There is still a great need for novel macrocyclic peptides having specific binding affinity to Lys63-linked Ub chains.

### SUMMARY OF THE INVENTION

The present invention, in some embodiments, is based, at least in part, on the characterization of cyclic peptides and their chemically modified analogs, resulting in highly potent compounds that are cell permeable.

The present invention is directed to a cyclic peptide and methods of using same, such as for ameliorating, or treating Lys63 Ub-related diseases, such as, but not limited to, cancer, in a subject in need thereof.

The present invention is based, at least in part, on the findings that cyclic peptides bind Lys63-linked Ub chains with an affinity K_{D} at a nanomolar level. The present invention is further based, in part, on the surprising finding that cyclic peptides, e.g., Lys-63 Ub binders, as disclosed herein, inhibit DNA repair in a cancerous cell, which in turn, induce cell cycle arrest, apoptosis, or both, of the cancerous cell.

According to one aspect, there is provided cyclic peptide comprising the amino acid sequence: LLIWIGSSKNPYILCG (SEQ ID NO: 1) or a functional analog thereof having at least 80% homology or identity thereto.

According to another aspect, there is provided a dimeric cyclic peptide comprising the cyclic peptide disclosed herein.

According to another aspect, there is provided a pharmaceutical composition comprising the cyclic peptide disclosed herein; or the dimeric cyclic peptide disclosed herein, and an acceptable carrier.

According to another aspect, there is provided a method for ameliorating or treating a K63Ub-related disease in a subject in need thereof, the method comprising administering to the subject a therapeutically effective amount of any one of: (a) the cyclic peptide disclosed herein; (b) the dimeric cyclic peptide disclosed herein; (c) the pharmaceutical composition disclosed herein; and (d) any one of (a) to (c), thereby ameliorating or treating a K63Ub related disease in the subject.

In some embodiments, the cyclic comprises at least one cysteine residue substituting an amino acid residue of SEQ ID NO: 1.

In some embodiments, the cyclic peptide comprises an amino acid sequence selected from the group consisting of: CLIWIGSSKNPYILCG (SEQ ID NO: 2); LCIWIGSSKNPYILCG (SEQ ID NO: 3); LLCWIGSSKNPYILCG (SEQ ID NO: 4) LLICIGSSKNPYILCG (SEQ ID NO: 5); LLIWCGSSKNPYILCG (SEQ ID NO: 6); LLIWICSSKNPYILCG (SEQ ID NO: 7) LLIWIGCSKNPYILCG (SEQ ID NO: 8); LLIWIGSCKNPYILCG (SEQ ID NO: 9); LLIWIGSSKCPYILCG (SEQ ID NO: 10); LLIWIGSSKNCYILCG (SEQ ID NO: 11); LLIWIGSSKNPCILCG (SEQ ID NO: 12); and LLIWIGSSKNPYCLCG (SEQ ID NO: 13).

In some embodiments, the cyclic peptide further comprises at least one arginine reside.

In some embodiments, the at least one arginine reside is located at the C-terminus of the cyclic peptide.

In some embodiments, the cyclic peptide comprises an amino acid sequence selected from the group consisting of: CLIWIGSSKNPYILCGRR (SEQ ID NO: 15); CLIWIGSSKNPYILCRR (SEQ ID NO: 16); and CLIWIGSSKNPYILCR (SEQ ID NO: 17).

In some embodiments, the cyclic peptide comprises 14 to 20 amino acid residues.

In some embodiments, the amino acid at position one of the N terminus is conjugated to a cyclizing molecule.

In some embodiments, the cyclizing molecule comprises a halogen.

In some embodiments, the cyclizing molecule comprises any one of:

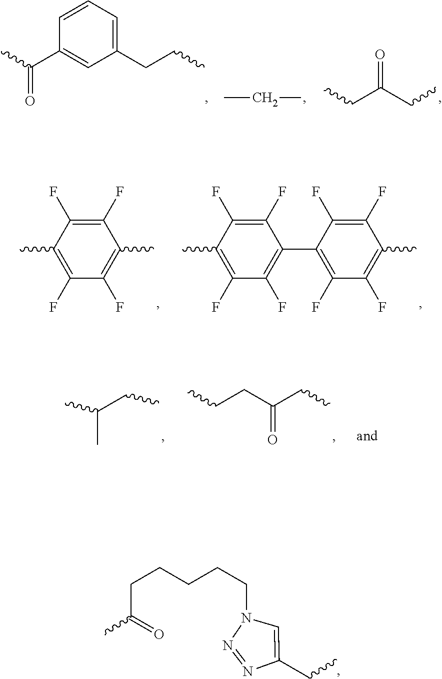

wherein each wavy bond represents an attachment point to the first amino acid residue or to the C-terminal amino acid, respectively.

In some embodiments, the cyclic peptide is chemically modified.

In some embodiments, the chemical modification is selected from the group consisting of: alkylation, arylation, addition of a thiol protecting group, and any combination thereof.

In some embodiments, the cyclic peptide is characterized by having: cell penetration capability, ubiquitin (Ub) binding capability, or any combination thereof. In some embodiments, the Ub is a polymeric Ub.

In some embodiments, the polymeric Ub comprises Ub monomers linked at their Lysine at position 63 (K63Ub).

In some embodiments, the cyclic peptide has increased affinity to Lys63-linked Ub chain, compared to a control Ub chain.

In some embodiments, the increased affinity is binding affinity with a dissociation constant (K_{D}) of 0.05-150 nM.

In some embodiments, the pharmaceutical composition is for use in the treatment of a K63Ub-related disease.

In some embodiments, the K63Ub-related disease is a cell proliferation-related disease.

In some embodiments, the cell-proliferation related disease comprises cancer.

In some embodiments, the ameliorating or treating comprises: increasing an amount of fragmented DNA in a cell of the subject, increasing an amount, rate, or both, of cell apoptosis in the subject, or any combination thereof.

In some embodiments, the cell is a caner or cancerous cell.

In some embodiments, the method further comprises administering to the subject a therapeutically effective amount of an anticancer agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B include schematic workflow images describing the strategy for the development of macrocyclic peptides against the Lys63-linked Ub chain. (1A) is a scheme of the general discovery process for macrocyclic peptides for Di-Ub chains using chemical protein synthesis and Random Non-standard Peptides Integrated Discovery (RaPID) approach to affect specific biological functions. (1B) is a schematic presentation of RaPID method for the identification of the novel binder 1 (CP1) for Lys63-linked Di-Ub.

FIGS. 2A-2E include synthesis scheme images and graphs demonstrating the chemical synthesis of cyclic peptides and their affinity screening for Lys63-linked Di-Ub. (2A) is a schematic general presentation for screening of the cyclic peptide library, chemically prepared to employ Fmoc-SPPS. (2B) and (2C) include synthetic scheme images demonstrating the preparation of cyclic peptides with Cys residue at various positions. (2D) is a bar chart demonstrating the binding affinity of cyclic peptides to Lys63-linked Di-Ub, normalized to the affinity of 1 (CP1; SEQ ID NO: 1). (2E) is a scatter plot binding curve of FITC-labeled cyclic peptide 1 (CP1-FITC). Determining the K_{D} (95.8±2.3 nM) of CP1-FITC. All measurements were performed in triplicates and at least three biological replicates. Error bars represent standard error.

FIGS. 3A-3B include a synthetic scheme, a table and a graph demonstrating the chemical synthesis of modified cyclic peptides containing Cys at position 1 (Cys1) and their affinity screening for Lys63-linked Di-Ub. (3A) is a schematic presentation for alkylation and arylation of Cys1. The alkylation and arylation substituents of cyclic peptide 2 are presented in the table. (3B) is a bar chart showing the binding affinity of the derivatives of cyclic peptides 2 to Lys63-linked Di-Ub, normalized to the affinity of 1 (CP1). All measurements were performed in triplicates and at least three biological replicates. Error bars represent standard error.

FIGS. 4A-4K include a synthetic scheme, immunofluorescence images, western blot images and graphs showing the disruption of DNA damage repair activity by cyclic peptides. (4A) is an illustration of the chemical composition of cyclic peptide 2 and its TAMRA labeled 26. (4B) demonstrates representative confocal images of 26 in live U2OS cells. Scale bars 20 μm. (4C) is a western blot analysis of lysates from U2OS cells treated with 33 (a scrambled sequence of cyclic peptide 2) and 2 (upper panels). H2AX was used as a loading control (lower panels). Representative of three independent experiments. (4D) is a bar graph showing the quantified relative γ-H2AX signals from C. (4E) is a western blot analysis of lysates from U2OS cells treated with 2 and 20 (upper panels). H2AX was used as a loading control (lower panels). Representative image of three independent experiments. (4F) is a bar graph demonstrating quantified relative γ-H2AX signal from E. (4G) exhibit immunofluorescence images of DNA damage, that was visualized by a “comet-like” vista green signal from the DNA of individual cells, analyzed over 100 cells in two independent experiments. (4H) is a bar graph demonstrating quantified relative tail moment (mean±SEM) of images from G. (4I) is an image of western blot using anti-flag antibody (upper panels) and anti-ubiquitin (lower panels) from anti-flag immunoprecipitated 293T cell lysates treated with and without 2 and transfected with RFN168 wt or mutant. (4J) is a bar graph showing the cell cycle distribution of HeLa cells treated with and without 2 after 72 and 96 h. From two independent flow cytometry experiments (>15,000 cells each condition). (4K) is a bar graph demonstrating the relative population of annexin V-FITC (apoptotic) cells after 96 h, from two independent flow cytometry experiments (>20,000 cells for each condition). Data were plotted as mean±SD (unless mentioned) and * is P<0.05, ** is P<0.005, *** is P<0.0005 and NS is non-significant.

FIGS. 5A-5D include schematic illustration, western blot images and graphs showing the identification of ubiquitinated proteins that bind peptides 31 and 38. (5A) is a schematic workflow of sample preparation and analysis by proteomics using biotin-conjugated cyclic peptides. (5B) is western blot analysis demonstrating the Ub chains that were pulled down by peptides 31 and 38 and detected by antibodies for Lys63 and Lys48-linked Ub chains. (5C) is a volcano plot showing the differentially enriched proteins. Proteins involved in processes mediated by Lys63-linked Ub are highlighted as: protein transport (blue), DNA repair (red), histone modification (green), and cell cycle (purple). (5D) is a horizontal bar graph showing the gene ontology (GO) analysis of genes from C with at least 3-fold enrichment compared to scramble control 38. The experiment was performed in triplicate.

FIGS. 6A-6B include synthetic scheme images and histograms demonstrating the synthesis of biotinylated-Lys63 linked Di-Ub. (6A) Schematic representation for the synthesis of biotin-Lys63 linked Di-Ub. (6B) Analytical HPLC and mass of the purified biotinylated-Lys63 linked Di-Ub with the observed mass 17592.7±0.2 Da (calcd. 17592.8, average isotopes).

FIG. 7 includes a bar graph showing the RaPID selection of three new libraries with mCIBz as an initiator and its real-time PCR results. (Negative=50% naked M280 streptavidin beads+50% Ub1-attached M280 streptavidin beads; Positive=Lys63 linked Di-Ub-attached M280 streptavidin beads).

FIG. 8 includes a schematic illustration showing a general presentation for screening the peptides using the fluoresce-based competitive assay.

FIGS. 9A-9B include synthetic scheme images and histograms demonstrating synthesis of CP1. (9A) is a schematic presentation of cyclic peptide synthesis. (9B) is a histogram of HPLC and mass analysis of cyclic peptide 1, CP1 with the observed mass 1891.5±0.1 Da (calcd. 1891.6 Da, average isotopes).

FIGS. 10A-10B include synthetic schemes and histograms showing the synthesis of different analogs of 1. (10A) is a schematic presentation of cysteine mutated cyclic peptides. (10B) demonstrates histograms of high performance liquid chromatography (HPLC) and mass spectrometry (MS) analysis of each cyclic peptide analog.

FIG. 11 includes a histogram of HPLC-MS analysis of cyclic peptide 15, CP1-L1C-CH3, with the observed mass of 1895.5±0.1 Da (calcd. 1895.3 Da, average isotopes).

FIG. 12 includes a histogram of HPLC-MS analysis of cyclic peptide 16, CP1-L1C-CH2C6H5, with the observed mass of 1971.5±0.2 Da (calcd. 1971.3 Da, average isotopes).

FIG. 13 includes a histogram of HPLC-MS analysis of cyclic peptide 17, CP1-L1C-CH2CONH2, with the observed mass of 1938.5±0.2 Da (calcd. 1938.3 Da, average isotopes).

FIG. 14 includes a histogram of HPLC-MS analysis of cyclic peptide 18, CP1-L1C-CH2C10H7, with the observed mass of 2021.5±0.2 Da (calcd. 2021.3 Da, average isotopes).

FIG. 15 includes a histogram of HPLC-MS analysis of cyclic peptide 19, CP1-L1C-CH2C10H7, with the observed mass of 2069.6±0.2 Da (calcd. 2069.3 Da, average isotopes).

FIG. 16 includes a histogram of HPLC-MS analysis of cyclic peptide 20, CP1-L1C-C6F5, with the observed mass of 2047.5±0.2 Da (calcd. 2047.3 Da, average isotopes).

FIG. 17 includes a histogram of HPLC-MS analysis of cyclic peptide 21, CP1-L1C-C10F9, with the observed mass of 2195.6±0.2 Da (calcd. 2195.3 Da, average isotopes).

FIGS. 18A-18B include synthetic scheme images and histograms showing the synthesis of CP1-FITC cyclic peptide. (18A) Schematic presentation for the synthesis of CP1-FITC. (18B) (i) HPLC-MS analysis of FITC labeled cyclic peptide, CP1-FITC with the observed mass of 2492.7±0.1 Da (calcd. 2492.5 Da, average isotopes). (ii) HPLC-MS analysis of TAMRA labeled cyclic peptide, CP1-TAMRA with the observed mass of 2546.5±0.1 Da (calcd. 2546.5 Da, average isotopes).

FIG. 19 includes a histogram showing HPLC-MS analysis of cyclic peptide 23 with the observed mass of 2214.5±0.1 Da (calcd. 2214.5 Da, average isotopes).

FIG. 20 includes a histogram demonstrating HPLC-MS analysis of cyclic peptide 24 with the observed mass of 2126.6±0.2 Da (calcd. 2126.5 Da, average isotopes).

FIG. 21 includes a histogram demonstrating HPLC-MS analysis of cyclic peptide 25 with the observed mass of 2607.5±0.1 Da (calcd. 2607.5 Da, average isotopes).

FIG. 22 includes a histogram demonstrating HPLC-MS analysis of cyclic peptide 26 with the observed mass of 2536.9±0.2 Da (calcd. 2537.1 Da, average isotopes).

FIGS. 23A-23B include synthetic scheme images and histograms showing the synthesis of TAMRA labeled CP1-L1C-C6F5 cyclic peptide. (23A) is a schematic presentation for the synthesis of TAMRA labeled CP1-L1C-C6F5. (23B) HPLC-MS analysis of cyclic peptides in which the peak corresponds to (i) 26 with the observed mass of 2537.1±0.2 Da (calcd. 2537.2 Da, average isotopes) (ii) 27 with the observed mass of 2702.6±0.2 Da (calcd. 2702.5 Da, average isotopes). Where TAMRA=Tetramethylrhodamine-5-maleimide.

FIG. 24 includes graphs showing the binding affinity of cyclic peptides 1 and 2 against Ub chains with different linkages and lengths. (i) Binding of cyclic peptide 1 on Lys11 linked Di-Ub and observed no binding by SPR. (ii) Binding of cyclic peptide 1 on Lys29 linked di-Ub. (Red: original trace, Black: fitting curve) (iii) Relative binding of cyclic peptides 1 and 2 on linear di-Ub. (iv) Relative binding of cyclic peptides 1 and 2 on Lys48 linked Di-Ub. (v) Relative binding of cyclic peptides 1 and 2 on Lys48 linked Tetra-Ub.

FIG. 25 includes a histogram of HPLC-MS analysis of cyclic peptide 28 with the observed mass of 2553.7±0.1 Da (calcd. 2553.5 Da, average isotopes).

FIGS. 26A-26B include synthetic scheme images and histogram showing (26A) the synthesis of FITC labeled cyclic peptide 29 and the (26B) HPLC-MS analysis of cyclic peptide 29 with the observed mass of 2482.6±0.1 Da (calcd. 2482.5 Da, average isotopes).

FIG. 27 includes a graph showing the binding of cyclic peptide 1 against Lys63 linked Di-Ub by SPR.

FIG. 28 includes a curve representing the binding of CP1-FITC to Lys63 linked Di-Ub. Using Y=Bmax*X/(K_{D}+X) formula, the determined K_{D} value=95.8±2.3 nM. All measurements were performed in triplicates.

FIG. 29 includes a curve representing the binding of CP1-TAMRA to Lys63 linked Di-Ub. Using Y=Bmax*X/(K_{D}+X) formula, the determined K_{D} value=101.9±3.6 nM. All measurements were performed in triplicates.

FIG. 30 includes a curve representing the binding of 2-FITC to Lys63 linked Di-Ub. Using Y=Bmax*X/(K_{D}+X) formula, the determined K_{D} value=43.2±4 nM. All measurements were performed in triplicates.

FIG. 31 includes a curve representing the binding of 2-TAMRA to Lys63 linked Di-Ub. Using Y=Bmax*X/(K_{D}+X) formula, the determined K_{D} value is 47.4±5.9 nM. All measurements were performed in triplicates.

FIGS. 32A-32B include synthetic scheme images and histograms demonstrating (32A) the synthesis of Biotin-PEG₆ coupled cyclic peptide 31. (32B) HPLC-MS analysis of cyclic peptide 31 with the observed mass of 2757.5±0.1 Da (calcd. 2757.6 Da, average isotopes).

FIGS. 33A-33C include synthetic scheme images, histogram and bar-chart demonstrating the synthesis of scrambled cyclic peptide 33. (33A) is a schematic presentation for the synthesis of cyclic peptide 33. (33B) is a histogram of HPLC-MS analysis of cyclic peptide 33 with the observed mass of 1881.4±0.1 Da (calcd. 1881.3 Da, average isotopes). (33C) is a bar chart showing the relative binding of cyclic peptide 33 and mJ08-L8W on Lys63 linked Di-Ub.

FIGS. 34A-34B include synthetic scheme images and histogram showing the synthesis of Biotin-PEG6 coupled scrambled cyclic peptide 38. (34A) is a schematic presentation for the synthesis of biotinylated cyclic peptide 38. (34B) is a histogram of HPLC-MS analysis of cyclic peptide 38 with the observed mass of 2757.1±0.1 Da (calcd. 2757.6 Da, average isotopes).

FIG. 35A-35L include fluorescent representative images of delivery of cyclic peptide 26 (2-TAMRA) and 29 (2-FITC) to live U2OS cells. CLSM images of 26, DMSO, and 29 treated cells; (35A-35D), (35E-35H), and (35I-35L), respectively. 35A, 35E) (TAMRA, red channel). 35I) (FITC, green channel). 35B, 35F, 35J) Hoechst (blue channel). 35C, 35G) TAMRA and Hoechst channels combined. 35K) FITC and Hoechst channels combined. 35D, 35H, 35L) Bright field channel. (Scale bars 20 μm). The experiment was repeated twice in duplicate each time at 2 μM.

FIG. 36A-36B include a western blot image and a graph demonstrating: (36A) western blot analysis of lysates from HeLa cells treated with 2 and 20 (upper panels). H2AX was used as a loading control (lower panels). Representative of three independent experiments. (36B) is a bar graph demonstrating quantified relative γ-H2AX signals from A. Data were plotted as mean±SD and * is P<0.05, ** is P<0.005.

FIGS. 37A-37B include dot plot graphs demonstrating flow cytometry analysis. Cells were double-stained with Annexin V-FITC and PI, subsequently analyzed by CYTEK Aurora flow cytometer. Two independent experiments were performed; HeLa cells treated with DMSO (37A) and cyclic peptide 2 (37B) for 96 h, and representative dot plots were shown here (>20,000 cells each condition).

FIGS. 38A-38C include histograms demonstrating flow cytometry analysis. Cell cycle distribution of HeLa cells treated with DMSO (38A) and cyclic peptide 2 for 72 (38B) and 96 h (38C). The representative heat plots showed a relative population of cells at G1, S, and G2/M phases in green, red, and blue boundaries, respectively (>15,000 cells each condition). Representative histograms of two independent experiments.

FIG. 39 includes an image of proteomics analysis after pull-down from streptavidin beads of Lys63-linked Ub modified protein components in U2OS cell lysate. STRING network of DDR proteins (identified by their gene names) enriched by 31. Data were shown for at least 3-fold enrichment compared to scramble control 38. The experiment was performed in triplicate.

FIGS. 40A-40C include peptide illustrations, vertical bar graphs, and a photograph showing argenylation in the cyclic peptide sequence for the improvement of physical and biological properties of a parent peptide (2). (40A) The chemical library of 2 with arginine conjugation (39-42), was chemically prepared by employing Fmoc-SPPS. (40B) The binding affinity of cyclic peptides to Lys63-linked Di-Ub, normalized to the affinity of 1. Data were plotted as mean±SEM for n=3 biologically independent experiments and each in triplicates. (40C) Western blot analysis of lysates from HeLa cells treated with 2 and its argenylated derivatives (39-42). H2AX was used as a loading control which was performed in a different blot. Representative image of n=4 independent experiments and quantified relative γ-H2AX signal. Data are presented as mean values±SD.

FIGS. 41A-41E include peptide illustrations, vertical bar graphs, and photographs showing cyclic peptides with different linkers for cyclization to improve biological properties of 40. (41A) The chemical library of 43-50 was chemically prepared by employing Fmoc-SPPS. (41B) The binding affinity of cyclic peptides to Lys63-linked Di-Ub, normalized to the affinity of 1. Data were plotted as mean±SD for n=2 biologically independent experiments and each in triplicates. (41C) Western blot analysis of lysates from HeLa cells treated with 40 and its derivatives with different linkers, 43-46. H2AX was used as a loading control which was performed in a different blot. Representative image of n=4 independent experiments and quantified relative γ-H2AX signal. Data are presented as mean values±SEM. (41D) Western blot analysis of lysates from HeLa cells treated with 43 (the best compound from our last biological screening in c) and other derivatives with different linkers, 47-49. H2AX was used as a loading control which was performed in a different blot. Representative image of n=4 independent experiments and quantified relative γ-H2AX signal. Data are presented as mean values±SEM. e Western blot analysis of lysates from HeLa cells treated with 43 and cleavable derivative in presence of GSH, 50.

FIGS. 42A-42H include exemplary synthetic schemes demonstrating the synthesis of cyclic peptides of the invention, such as peptides 39-42 (42A), peptide 43 (42B), peptide 44 (42C), peptide 45 (42D), peptide 46 (42E), peptide 50 (42F), peptide 48 (42G) and peptide 49 (42H).

FIG. 43 includes exemplary synthetic schemes demonstrating the synthesis of peptide 47 and a general synthetic scheme of a solid-phase synthesis of an amino acid sequence including propargyl glycine, which is a precursor for peptides 47-49.

### DETAILED DESCRIPTION OF THE INVENTION

The present invention, in some embodiments, is directed to a cyclic peptide and methods of using same, such as for binding Ub of a cell, or for ameliorating, or treating a disease in a subject in need thereof. The present invention in some embodiments, combines the powerful random non-standard peptides integrated discovery (RaPID) method, with the inventors ability to synthesize specific Ub chains, such as Ub chains linked via their Lysine 63 residue (Lys63-linked Ub), to discover new cyclic peptides that bind specifically, and with high affinity of nanomolar level, to Lys63-linked Ub. The present invention, in some embodiments, is further based, at least in part, on the capability of these new cyclic peptides to affect diverse cellular processes, including DNA repair and apoptosis, highlighting these cyclic peptides as a therapeutic strategy to diseases in which Lys63 ubiquitination is dysregulated, such as, but not limited to, cancer.

### Cyclic Peptides

According to some embodiments, the invention is directed to a peptide.

In some embodiments, the peptide is capable of penetrating a cell (e.g. a cancer cell), binding to ubiquitin, or a combination thereof.

In some embodiments, a peptide of the invention comprises or consists of the amino acid sequence: LLIWIGSSKNPYILCG (SEQ ID NO: 1).

In some embodiments, a peptide of the invention comprises or consists of the amino acid sequence: CLIWIGSSKNPYILCG (SEQ ID NO: 2).

In some embodiments, a peptide of the invention comprises or consists of the amino acid sequence: LCIWIGSSKNPYILCG (SEQ ID NO: 3).

In some embodiments, a peptide of the invention comprises or consists of the amino acid sequence: LLCWIGSSKNPYILCG (SEQ ID NO: 4).

In some embodiments, a peptide of the invention comprises or consists of the amino acid sequence: LLICIGSSKNPYILCG (SEQ ID NO: 5).

In some embodiments, a peptide of the invention comprises or consists of the amino acid sequence: LLIWCGSSKNPYILCG (SEQ ID NO: 6).

In some embodiments, a peptide of the invention comprises or consists of the amino acid sequence: LLIWICSSKNPYILCG (SEQ ID NO: 7).

In some embodiments, a peptide of the invention comprises or consists of the amino acid sequence: LLIWIGCSKNPYILCG (SEQ ID NO: 8).

In some embodiments, a peptide of the invention comprises or consists of the amino acid sequence: LLIWIGSCKNPYILCG (SEQ ID NO: 9).

In some embodiments, a peptide of the invention comprises or consists of the amino acid sequence: LLIWIGSSKCPYILCG (SEQ ID NO: 10).

In some embodiments, a peptide of the invention comprises or consists of the amino acid sequence: LLIWIGSSKNCYILCG (SEQ ID NO: 11).

In some embodiments, a peptide of the invention comprises or consists of the amino acid sequence: LLIWIGSSKNPCILCG (SEQ ID NO: 12).

In some embodiments, a peptide of the invention comprises or consists of the amino acid sequence: LLIWIGSSKNPYCLCG (SEQ ID NO: 13).

In some embodiments, a peptide of invention comprises a functional analog of any one of SEQ ID Nos: 1-17.

In some embodiments, a functional analog is characterized by having at least 70%, 80%, 85%, 90%, 95% or 99% homology or identity to any one of SEQ ID Nos: 1-17, or any value and range therebetween. Each possibility represents a separate embodiment of the invention. In some embodiments, a functional analog is characterized by having 70-100%, 80-100%, 85-100%, 90-100%, 95-100% or 97-100% to any one of SEQ ID Nos: 1-17. Each possibility represents a separate embodiment of the invention.

The term “functional analog,” as used herein, generally refers to any peptide characterized by having functionally being essentially the same as a cyclic peptide disclosed herein. In some embodiments, a functional analog as disclosed herein binds Lys63Ub with a binding affinity being at least 50%, 60%, 70%, 80%, 90%, 95%, or 99% of the binding affinity of the peptide of the invention as disclosed herein, or any value and range therebetween. Each possibility represents a separate embodiment of the invention. In some embodiments, a functional analog is characterized by having 70-100%, 80-100%, or 90-100% homology or identity to any one of SEQ ID Nos: 1-17. Each possibility represents a separate embodiment of the invention.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percentage identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

The term “analog” as used herein, refers to a polypeptide that is similar, but not identical, to the peptide of the invention that still is capable of binding Ub, such as Lys63 Ub dimers, oligomers, or polymers. An analog may have deletions or mutations that result in an amino acids sequence that is different than the amino acid sequence of the peptide of the invention. It should be understood that all analogs of the peptide of the invention would still be capable of binding Ub. Further, an analog may be analogous to a fragment of the peptide of the invention, however, in such a case the fragment must comprise at least 14 consecutive amino acids of the peptide of the invention.

In some embodiments, the peptide of the invention is linear or cyclic.

As defined herein, the amino acid sequence of a peptide of the invention is cited from the N-terminus to the C-terminus. In some embodiments, amino acid residue positioned at the N-terminus of a peptide is located at the first position of the peptide. In some embodiments, a cited position of a given amino acid residue within a cyclic peptide is referred to, based on the position of the amino acid residue in the linear form of the peptide.

In some embodiments, the N-terminus of a cyclic peptide as disclosed herein is defined as the “endocyclic end or position”.

In some embodiments, the N-terminal amino acid of the cyclic peptide as disclosed herein is located in the endocyclic position of the cyclic peptide.

In some embodiments, the C-terminus of a cyclic peptide as disclosed herein is defined as the “exocyclic end or position”.

In some embodiments, the C-terminal amino acid of the cyclic peptide as disclosed herein is located in the exocyclic position of the cyclic peptide.

In some embodiments, the peptide further comprises a positively charged amino acid residue. In some embodiments, a positively charged amino acid residue is positively charged in physiological condition, e.g., pH, temperature, osmolarity, etc., such as, but no limited to a human cell and/or body.

In some embodiments, the peptide further comprises at least one amino acid residue being selected from: arginine, lysine, histidine, or any combination thereof.

In some embodiments, at least one amino acid residue comprises a plurality of amino acid residues. In some embodiments, a plurality comprises any integer being equal to or greater than 2. In some embodiments, a plurality comprises 2 to 8, 2 to 6, or 2 to 4 amino acid residues. Each possibility represents a separate embodiment of the invention.

In some embodiments, the plurality of amino acid residues are covalently bound to one another. In some embodiments, the plurality of amino acid residues are bound to one another by a peptide bond.

In some embodiments, at least one amino acid residue or plurality thereof is or are positioned in the endocyclic position or end of the peptide.

In some embodiments, at least one amino acid residue or plurality thereof is or are positioned in the exocyclic position or end of the peptide.

In some embodiments, at least one amino acid residue comprises or consists of arginine.

In some embodiments, the peptide further comprises at least one arginine residue.

In some embodiments, the peptide further comprises a plurality of arginine residues.

In some embodiments, the peptide further comprises 2 arginine residues. In some embodiments, the peptide further comprises 2 arginine residues bound to one another by a peptide bond.

In some embodiments, at least one arginine or plurality thereof is or are positioned in the endocyclic position or end of the peptide.

In some embodiments, at least one arginine or plurality thereof is or are positioned in the exocyclic position or end of the peptide.

In some embodiments, the peptide further comprises 2 arginine residues bound to one another by a peptide bond, and being located in the endocyclic position of the peptide.

In some embodiments, the peptide further comprises 2 arginine residues bound to one another by a peptide bond, and being located in the exocyclic position of the peptide.

In some embodiments, a peptide of the invention comprises or consists of the amino acid sequence: CRIWIGSSKNPYILCG (SEQ ID NO: 14).

In some embodiments, a peptide of the invention comprises or consists of the amino acid sequence: CLIWIGSSKNPYILCGRR (SEQ ID NO: 15).

In some embodiments, a peptide of the invention comprises or consists of the amino acid sequence: CLIWIGSSKNPYILCRR (SEQ ID NO: 16).

In some embodiments, a peptide of the invention comprises or consists of the amino acid sequence: CLIWIGSSKNPYILCR (SEQ ID NO: 17).

In some embodiments, the peptide comprises 14-20 amino acids. In some embodiments, at least 14 amino acid residues comprises at least 14, 15, 16, 17, 18, 19, or 20 amino acid residues, or any value and range therebetween. Each possibility represents a separate embodiment of the invention. In some embodiments, 14-20 amino acid residues comprises 14-19, 14-18, 14-17, 14-16, 14-15, 15-20. 15-19, 15-18, 15-17, 15-16, 16-20, 16-19, 16-18, 16-17, 17-20, 17-19, 17-18, 18-20, 18-19, or 19-20 amino acid residues. Each possibility represents a separate embodiment of the invention.

In some embodiments, the peptide of the invention comprises at least one amino acid substitution compared to SEQ ID NO: 1.

In some embodiments, the amino acid substitution comprises a substitution to a cysteine residue.

In some embodiments, the substituting cysteine residue is chemically modified. In some embodiments, the substituting cysteine residue is functionalized.

In some embodiments, the peptide of the invention comprises at least one chemical modification.

In some embodiments, the chemical modification comprises a protection or protective group. In some embodiments, the protection or protective group comprises a thiol protecting group. In some embodiments, the thiol protecting group comprises acetamidomethyl (Acm) group.

A “thiol protecting group” is well known in the art and includes those described in detail in Protecting Groups in Organic Synthesis, T. W. Greene and P. G. M. Wuts, 3.sup.rd edition, John Wiley & Sons, 1999, the entirety of which is incorporated herein by reference. Examples of protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of ester groups include formates, acetates, proprionates, pentanoates, crotonates, and benzoates. Specific examples of ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl groups include trimethylsilyl, triethylsilyl, t-butyldimethylsilyl, t-butyldiphenylsilyl, triisopropylsilyl ether, and other trialkylsilyl ethers. Examples of alkyl groups include methyl, benzyl, p-methoxybenzyl, 3,4-dimethoxybenzyl, trityl, t-butyl, and allyl ether, or derivatives thereof. Examples of arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In some embodiments, at least one chemical modification is selected from: alkylation, arylation, oxidation, or any combination thereof.

In some embodiments, the functionalized cysteine as disclosed herein is conjugated to a carbon chain. In some embodiments, the peptide of the invention comprises a functionalized cysteine residue being conjugated to a carbon chain. In some embodiments, a carbon chain comprises one or more carbons. In some embodiments, a carbon chain comprising one or more carbons comprises at least 2, at least 3, at least 4, or at least five carbons, or any value and range therebetween. Each possibility represents a separate embodiment of the invention. In some embodiments, a carbon chain comprising one or more carbons comprises 2-3, 2-4, 2-5, 3-4, 3-5, or 4-5 carbons. Each possibility represents a separate embodiment of the invention. In some embodiments, amino acid residues of a polypeptide of the invention as mentioned above are functionalized by conjugation to a methyl group, ethyl group, propyl group, butyl group, or any combination thereof.

In some embodiments, alkylation or arylation comprises the conjugation of one of the groups: CH₃, CH₂C₆H₅, CH₂CONH₂, CH₂C₁₀H₇, CH₂C₉H₅O₂, CH₂C₁₀H₇O₃, C₆F₅, C₁₀F₉, or a combination thereof. In some embodiments, alkylation or arylation comprises the conjugation of one of the groups: CH₃, CH₂C₆H₅, CH₂CONH₂, CH₂C₁₀H₇, CH₂C₉H₅O₂, CH₂C₁₀H₇₀₃, C₆F₅, C₁₀F₉, or a combination thereof, to the first residue in SEQ ID NO: 2 (Cys1).

In some embodiments, the peptide is conjugated to a fluorophore. In some embodiments, the peptide is conjugated to a fluorophore selected from: Fluorescein-5-isothiocyanate (FITC) and carboxytetramethylrhodamine (TAMRA). In some embodiments, conjugated peptide-fluorophore is used to trace it in a cell, for assessment of cell permeability, or for screening of additional unlabeled peptides that compete with the peptide-fluorophore for Ub binding.

In some embodiments, a peptide of the invention is capable of binding ubiquitin (Ub). In some embodiments, a peptide of the invention has specific binding affinity to ubiquitin (Ub). In some embodiments, a peptide of the invention has increased binding affinity to a ubiquitin molecule, compared to a control.

As used herein, the term “Ubiquitin” refers to the regulatory protein which is added to other proteins by means of post translational modification. In some embodiments Ub is a dimeric Ub (Di-Ub). In some embodiments, Ub is a polymeric Ub (poly-Ub). In some embodiments, a polymeric Ub comprises at least 2, at least 3, at least 4, or at least 5 Ub monomers, or any value and range therebetween. Each possibility represents a separate embodiment of the invention. In some embodiments, a polymeric Ub comprises 2-3, 2-4, 2-5, 3-4, 3-5, or 4-5 Ub monomers. Each possibility represents a separate embodiment of the invention. In some embodiments, a Ub dimer comprises two Ub monomers linked to one another at Lys residue at position 63 (Lys63 Di-Ub).

In some embodiments, a peptide of the invention has a greater binding affinity to Lys63-linked Ub compared to other Ub dimers, oligomers, or polymers, such as, but not limited to, any one of Lys11-linked Ub, Lys29-linked Ub, and Lys48-linked Ub. In some embodiments, a peptide of the invention has a greater binding affinity to Lys63-linked Di-Ub compared to any one of: Lys11-linked Di-Ub, Lys29-linked Di-Ub, and Lys48-linked Di-Ub.

In some embodiments, a control Ub chain is devoid of Lys63Ub. In some embodiments, a control Ub chain does not comprise Lys63 Ub. In some embodiments, a control Ub chain comprises a Ub chain (e.g., polymer) wherein Ub monomers of the Ub chain are not linked to one another via Lys63. In some embodiments, a control Ub chain comprises a Ub chain (e.g., polymer) wherein Ub monomers of the Ub chain are linked to one another via Lys11, Lys29, Lys48, or any combination thereof.

In some embodiments, a Ub is further conjugated to a biotin tag. In some embodiments, Lys-63 Di-Ub conjugated to biotin is used to screen peptides that selectively bind Lys-63 Di-Di-Ub. In some embodiments, screened peptide(s) comprise cyclic peptide(s).

In some embodiments, a peptide of the invention is capable of binding to Lys63 Di-Ub in vitro, in vivo, ex vivo, or any combination thereof. In some embodiments, a peptide of the invention is capable of penetrating a cell. In some embodiments, the peptide requires no additional elements to penetrate a cell. In some embodiments, the peptide may be further formulated with other elements for enhancing cell penetration. In some embodiments, the peptide may be used as a carrier or vehicle to carry other elements into a cell.

In some embodiments, a peptide of the invention or a functional analog thereof, has Lys63 Di-Ub binding affinity with a KD of 0.05-1 nM, 0.5-5 nM, 1-10 nM, 5-15 nM, 10-20 nM, 15-30 nM, 20-40 nM, 35-50 nM, 45-60 nM, 55-70 nM, 65-80 nM, 75-90 nM, 85-95 nM, 90-120 nM, 100-500 nM, 250-750 nM, 0.7-1.5 μM, 1-5 μM, 4-10 μM, 8-20 μM, 1 nM to 1 μM, or 15-40 μM. Each possibility represents a separate embodiment of the invention. In some embodiments, a peptide of the invention, or a functional analog thereof, has Ub binding affinity with KD of 0.1 nM at most, 0.5 nM at most, 1 nM at most, 5 nM at most, 10 nM at most, 20 nM at most, 30 nM at most, 40 nM at most, 50 nM at most, 60 nM at most, 70 nM at most, 80 nM at most, 90 nM at most, 100 nM at most, 110 nM at most, 150 nM at most, 250 nM at most, 500 nM at most, 750 nM at most, 1,500 nM at most, 1 μM at most, 5 μM at most, 10 μM at most, 15 μM at most, 20 μM at most, or 30 μM at most, or any value and range therebetween. Each possibility represents a separate embodiment of the invention.

The present invention encompasses derivatives of the peptide of the invention. The term “derivative” or “chemical derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, amides (e.g. —CONH2), methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine (O) may be substituted for lysine.

In addition, a peptide derivative can differ from the natural sequence of the peptide of the invention by chemical modifications including, but are not limited to, terminal-NH2 acylation, acetylation, or thioglycolic acid amidation, and by terminal-carboxlyamidation, e.g., with ammonia, methylamine, and the like. Peptides can be either linear, cyclic, or branched and the like, having any conformation, which can be achieved using methods known in the art. In some embodiments, the peptide of the invention further comprises any posttranslational modification (PTM) excluding mammalian naturally occurring PTM.

The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

The term “amino acid” as used herein means an organic compound containing both a basic amino group and an acidic carboxyl group. Included within this term are naturally occurring amino acids, modified, unusual, non-naturally occurring amino acids, as well as amino acids which are known to occur biologically in free or combined form but usually do not occur in proteins. Included within this term are modified and unusual amino acids, such as those disclosed in, for example, Roberts and Vellaccio (1983) The Peptides. 5:342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalanine (2Np), aminoheptanoic acid (Ahp), phenylglycine, 1-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-,3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAI).

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to an aspartic acid (D).

As used herein, the phrase “conservative substitution” also includes the use of a chemically derivatized residue in place of a non-derivatized residue provided that such peptide displays the requisite function as specified herein.

The peptide derivatives according to the principles of the present invention can also include side chain bond modifications, including but not limited to —CH₂—NH—, —CH₂—S—, —CH₂—S=0, OC—NH—, —CH₂—O—, —CH₂—CH₂—, S—C—NH—, and —CH═CH—, and backbone modifications such as modified peptide bonds. Peptide bonds (—CO—NH—) within the peptide can be substituted, for example, by N-methylated bonds (—N(CH₃)—CO—); ester bonds (—C(R) H—C—O—O—C(R)H—N); ketomethylene bonds (—CO—CH₂—); a-aza bonds (—NH—N(R)—CO—), wherein R is any alkyl group, e.g., methyl; carba bonds (—CH₂—NH—); hydroxyethylene bonds (—CH(OH)—CH₂—); thioamide bonds (—CS—NH); olefmic double bonds (—CH═CH—); and peptide derivatives (—N(R)—CH₂—CO—), wherein R is the “normal” side chain, naturally presented on the carbon atom. These modifications can occur at one or more of the bonds along the peptide chain and even at several (e.g., 2-3) at the same time.

The invention further includes peptides and derivatives thereof, which can contain one or more D-isomer forms of the amino acids. Production of retro-inverso D-amino acid peptides where at least one amino acid and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. Diastereomeric peptides may be highly advantageous over all L- or all D-amino acid peptides having the same amino acid sequence because of their higher water solubility, lower immunogenicity, and lower susceptibility to proteolytic degradation. The term “diastereomeric peptide” as used herein refers to a peptide comprising both L-amino acid residues and D-amino acid residues. The number and position of D-amino acid residues in a diastereomeric peptide of the preset invention may be variable so long as the peptide is capable of displaying the function of disclosed chimera of the invention.

In some embodiments, the peptide of the invention is cyclized. In some embodiments, cyclized is via a linkage between the first amino acid residue of the N terminus of the peptide of the invention and a C terminal amino acid. In some embodiments, the C terminal amino acid is positioned at the position ranging between 3 and 20, between 5 and 20, between 5 and 18, between 5 and 16, between 5 and 14, between 10 and 20, between 10 and 18, between 10 and 16, between 10 and 14, including any range between. In some embodiments, the linkage is a covalent bond. In some embodiments, the linkage is formed between (i) the N-terminus of the first amino acid residue and the side chain of the C-terminal amino acid; or (ii) between the side chain of the first amino acid residue and the side chain of the C-terminal amino acid. In some embodiments, the first amino acid residue is a cysteine residue, and the linkage is via a thio group of the first amino acid. In some embodiments, the covalent bond is via direct conjugation of the side-chains (i.e. without a spacer, such as an S—S bond between cysteine residues). In some embodiments, the covalent bond comprises a linkage between the cysteine residue (e.g. N-terminal amino group, or thio group thereof) and the C-terminal amino acid via a cyclizing molecule. In some embodiments, the first amino acid residue of the N terminus of a peptide of the invention (endocyclic position) is conjugated to a cyclizing molecule. In some embodiments, the first amino acid residue of the peptide (positioned at the N terminus or endocyclic position) and another amino residue of the peptide (positioned at the C terminus) are bound to one another, thereby resulting in a cyclic peptide. In some embodiments, the cyclizing molecule is bound to both the first amino acid residue and to another amino acid residue located at the C terminus. In some embodiments, the cyclizing molecule facilitates the binding of the first amino acid residue (located N terminus or endocyclic position) and the C terminal amino acid residue. In some embodiments, the cyclizing molecule is conjugated to both the first amino acid residue (located N terminus or endocyclic position) and the C terminal amino acid residue. As defined herein, a “C terminal amino acid residue” refers to an amino acid residue located in a position closer to the C terminal end (exocyclic position) of a linear peptide compared to the N terminal end of the peptide. In some embodiments, a C terminal amino acid residue is positioned 8 before last, 7 before last, 6 before last, 5 before last, 4 before last, 3 before last, 2 before last, 1 before last, or is the last amino acid residue in a linear peptide. Each possibility represents a separate embodiment of the invention. According to a non-limiting example, an amino acid residue at position 9 of a peptide comprising 16 amino acid residues is considered as a C terminal amino acid residue. A variety of methods are available for cyclizing a polypeptide (e.g., macrocyclization) as reviewed, for example by White and Yudin (2011).

In some embodiments, the cyclizing molecule is a linker. In some embodiments, the linker comprises an optionally substituted C₁-C₁₀ alkyl, alkyl-aryl, halo-aryl, halo-biaryl, alkyl-C(O)-alkyl, a heteroatom (e.g., O, N, NH, or S), —C(O)NH—, —C(O)O—, —C(O)—, —C(O)S—, —C(NH) NH—, —C(NH)O—, —C(NH)S—, —S—S—, —S—C(═O), Y—(C₀-10)alkyl-X—(C0-10)alkyl-Y, or a click reaction product including any combination thereof; wherein each X and Y is absent or is independently selected from a heteroatom, an oligomer, a click reaction product, oxo, —CONR′—, —CNNR′—, —CSNR′—, —NC(═O)O—, —NC(═S)O—, —NC(═S)N—, —SO2-, —SO—, —SR′, —C(═O)—, —OC(═O)—, —OC(═O)O—, —OC(═S)O—, and —OC(═S)N—. In some embodiments, the oligomer comprises between 2 and 15 repeating units. In some embodiments, the repeating unit is selected form alkylene oxide, a natural or an unnatural amino acid residue, an alpha-hydroxy carboxylic acid residue, including any copolymer and any combination thereof.

Click reactions are well-known in the art and comprise inter alia Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction etc.

Accordingly, the term “click reaction product” encompasses a moiety formed via a click reaction, wherein the click reaction is as described hereinabove. In some embodiments, the click reaction product comprises a product formed by any of: Michael addition of maleimide and thiol (resulting in the formation of a succinimide-thioether); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction, or any combination thereof.

In some embodiments, the cyclizing molecule comprises any one of

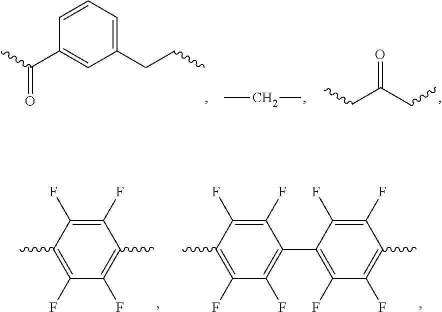

wherein the wavy bonds represent attachment points to the first amino acid residue and to the C-terminal amino acid, respectively.

In some embodiments, the linkage is between the side chain or N-terminal amino group of the first amino acid residue and the C-terminal amino acid via the cyclizing molecule, wherein the cyclizing molecule bound to the amino group of the C-terminal amino acid. Exemplary linkage is as depicted hereinbelow:

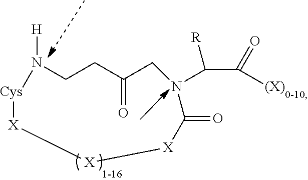

wherein each X independently represents an amino acid, R is a side chain of the C-terminal amino acid; the arrow points towards the attachment point of the cyclizing molecule to the amino group of the C-terminal amino acid; and the dashed arrow points towards the N-terminal amino group of the first amino acid residue. A skilled artisan will appreciate that the cyclizing molecule depicted above is only a non-limiting exemplary cyclizing molecule, and the peptide of the invention may include any cyclizing molecule, as disclosed herein.

Additional exemplary cyclizing molecules include but are not limited to:

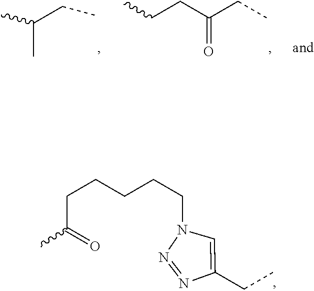

wherein the wavy bond represents an attachment point to the first amino acid residue (e.g. N-terminal amino group thereof), and wherein a dashed bond represents an attachment point to the amino group of the C-terminal amino acid.

In some embodiments, the cyclizing molecule comprises one or more halogen atoms selected from: Fluoride (F), Chlorine (CI), Bromide (Br), Iodine (I) and Astatine (At), or any combination thereof. Non-limiting examples for a cyclizing molecule comprising a halogen include, but are not limited to: chloroacetyl (ClAc), 3-(chloromethyl)benzoic acid (mCIBz), 4-(chloromethyl)benzoic acid (pCIBz), chloracetyl chloride, 3-chlorobenzoyl (3-ClBz), 4-chlorobenzoyl (4-ClBz) or C12SAc. In one embodiment, a cyclizing molecule comprising a halogen group is conjugated to the first amino acid residue of a polypeptide's N terminus and nucleophilicaly attacks a thiol group of a cysteine residue located at the C terminal end of the peptide, thereby resulting in a cyclic peptide.

In some embodiments, the cyclizing molecule is selected from: chloroacetyl (ClAc), 3-(chloromethyl)benzoic acid (mCIBz), 4-(chloromethyl)benzoic acid (pCIBz), chloracetyl chloride, 3-chlorobenzoyl (3-ClBz), 4-chlorobenzoyl (4-ClBz) or C12SAc.

In some embodiments, a cyclizing molecule as disclosed herein comprises a cyclizing precursor molecule. In some embodiments, the cyclizing precursor molecule comprises at least one halogen atom.

In some embodiments, the peptide is prepared using a cyclizing molecule comprising at least one halogen.

In some embodiments, the peptide of the invention is represented by a Formula 1:

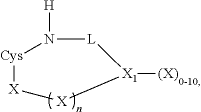

wherein each X independently represents an amino acid residue, wherein X₁ is the C-terminal amino acid; L represents the cyclizing molecule or is a bond; and n is an integer ranging between 0 and 16, between 1 and 16, between 2 and 16, between 3 and 16, between 4 and 16, between 5 and 16, including any range between.

In some embodiments, there is provided a dimeric peptide comprising the peptide of the invention. In some embodiments, there is provided a dimeric cyclic peptide comprising the cyclic peptide of the invention. In some embodiments, the dimeric peptide is a homodimer or a heterodimer. In some embodiments, the dimeric peptide is a homodimer. In some embodiments, the dimeric peptide comprises two monomeric peptides covalently linked to one another via cysteine residues. In some embodiments, the monomeric peptides are linked via a disulfide bond (—S—S—). In some embodiments, the monomeric peptides are linked via a linker, e.g., a carbon chain of one or more carbon atoms. In some embodiments, the monomeric peptides are linked via a CH₂ linker. In some embodiments, the monomeric peptides are linked via the following bond: —S—CH₂—S—.

### Peptides Synthesis

According to one embodiment, the peptide of the invention may be synthesized or prepared by any method and/or technique known in the art for peptide synthesis. According to another embodiment, peptide may be synthesized by a solid phase peptide synthesis method of Merrifield (see J. Am. Chem. Soc, 85:2149, 1964). According to another embodiment, the peptide of the invention can be synthesized using standard solution methods, which are well known in the art (see, for example, Bodanszky, M., Principles of Peptide Synthesis, Springer-Verlag, 1984).

In general, the synthesis methods comprise sequential addition of one or more amino acids or suitably protected amino acids to a growing peptide chain bound to a suitable resin. Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha, alpha)-dimethyl-3,5 dimethoxybenzyloxycarbonyl, o-nitrophenylsulfenyl, 2-cyano-t-butyloxycarbonyl, 9-fluorenylmethyloxycarbonyl (Fmoc) and the like. In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

In another embodiment, a peptide of the invention may be synthesized such that one or more of the bonds, which link the amino acid residues of the peptide are non-peptide bonds. In another embodiment, the non-peptide bonds include, but are not limited to, imino, ester, hydrazide, semicarbazide, and azo bonds, which can be formed by reactions well known to one skilled in the art.

The term “linker” refers to a molecule or macromolecule serving to connect different moieties of a peptide or a polypeptide. In one embodiment, a linker may also facilitate other functions, including, but not limited to, preserving biological activity, maintaining sub-units and domains interactions, and others.

In some embodiments, a peptide of the invention may be attached or linked to another molecule via a chemical linker. In some embodiments, attached is to be meant in a conjugation reaction. Chemical linkers are well known in the art and include, but are not limited to, dicyclohexylcarbodiimide (DCC), N-hydroxysuccinimide (NHS), maleiimidobenzoyl-N-hydroxysuccinimide ester (MBS), N-ethyloxycarbonyl-2-ethyloxy-1,2-dihydroquinoline (EEDQ), N-isobutyloxy-carbonyl-2-isobutyloxy-1,2-dihydroquinoline (IIDQ). In another embodiment, linkers may also be monomeric entities such as a single amino acid. In another embodiment, amino acids with small side chains are especially preferred, or a small polypeptide chain, or polymeric entities of several amino acids. In another embodiment, a polypeptide linker is fifteen amino acids long or less, ten amino acids long or less, or five amino acids long or less. In one embodiment, a linker may be a nucleic acid encoding a small polypeptide chain. In another embodiment, a linker encodes a polypeptide linker of fifteen amino acids long or less, ten amino acids long or less, or five amino acids long or less.

Recombinant technology may be used to express the peptide of the invention, and is well known in the art. In another embodiment, the linker may be a cleavable linker, resulting in cleavage of the peptide of the invention once delivered to the tissue or cell of choice. In such an embodiment, the cell or tissue would have endogenous (either naturally occurring enzyme or be recombinantly engineered to express the enzyme) or have exogenous (e.g., by injection, absorption or the like) enzyme capable of cleaving the cleavable linker.

In another embodiment, the linker may be biodegradable such that the polypeptide of the invention is further processed by hydrolysis and/or enzymatic cleavage inside cells. In one embodiment, tumor specifically-expressed proteases, can be used in the delivery of prodrugs of cytotoxic agents, with the linker being selective for a site-specific proteolysis. In some embodiments, a readily-cleavable group include acetyl, trimethylacetyl, butanoyl, methyl succinoyl, t-butyl succinoyl, ethoxycarbonyl, methoxycarbonyl, benzoyl, 3-aminocyclohexylidenyl, and the like.

The invention further encompasses a polynucleotide sequence comprising a nucleic acid encoding any of the peptides of the invention. In another embodiment, the nucleic acid sequence encoding the peptide is at least 70%, or alternatively at least 80%, or alternatively at least 90%, or alternatively at least 95%, or alternatively at least 99% homologous to the nucleic acid sequence encoding the nucleic acid sequence of the peptides of the invention or a derivative thereof, or any value and range therebetween. Each possibility represents a separate embodiment of the invention.

In some embodiments, the invention provides a polynucleotide encoding the peptide of the invention.

In some embodiments, the polynucleotide of the invention is ligated into an expression vector, comprising a transcriptional control of a cis-regulatory sequence (e.g., promoter sequence). In some embodiments, the cis-regulatory sequence is suitable for directing constitutive expression of the peptide of the invention. In some embodiments, the cis-regulatory sequence is suitable for directing tissue-specific expression of the polypeptide of the invention. In some embodiments, the cis-regulatory sequence is suitable for directing inducible expression of the peptide of the invention.

The term “polynucleotide” refers to a nucleic acid (e.g., DNA or RNA) sequence that comprises coding sequences necessary for the production of a peptide. In one embodiment, a polynucleotide refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA-dependent DNA polymerase. In one embodiment, the sequence can be subsequently amplified in vivo or in vitro using a DNA polymerase.

In one embodiment, “genomic polynucleotide sequence” refers to a sequence derived (or isolated) from a chromosome and, thus it represents a contiguous portion of a chromosome.

In one embodiment, “composite polynucleotide sequence” refers to a sequence which is at least partially complementary and at least partially genomic. In one embodiment, a composite sequence can include some exonal sequences required to encode the polypeptide of the invention, as well as some intronic sequences interposing therebetween. In one embodiment, the intronic sequences can be of any source, including of other genes, and typically may include conserved splicing signal sequences. In one embodiment, intronic sequences include cis-acting expression regulatory elements.

In some embodiments, a polynucleotide of the invention is prepared using PCR techniques, or any other method or procedure known to one of ordinary skill in the art.

In one embodiment, a polynucleotide of the invention is inserted into expression vectors (i.e., a nucleic acid construct) to enable expression of a recombinant peptide as disclosed herein. In one embodiment, the expression vector includes additional sequences which render this vector suitable for replication and integration in prokaryotes. In one embodiment, the expression vector includes additional sequences which render this vector suitable for replication and integration in eukaryotes. In one embodiment, the expression vector includes a shuttle vector which renders this vector suitable for replication and integration in both prokaryotes and eukaryotes. In some embodiments, cloning vectors comprise transcription and translation initiation sequences (e.g., promoters, enhancers) and transcription and translation terminators (e.g., polyadenylation signals).

In one embodiment, a variety of prokaryotic or eukaryotic cells can be used as host-expression systems to express the peptide of the invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In some embodiments, non-bacterial expression systems are used (e.g. mammalian expression systems) to express the peptide of the invention. In one embodiment, the expression vector is used to express the polynucleotide of the invention in mammalian cells.

In some embodiments, in bacterial systems, a number of expression vectors can be advantageously selected depending upon the use intended for the peptide expressed. In one embodiment, large quantities of peptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector may further include additional polynucleotide sequences that allow, for example, the translation of several proteins from a single mRNA such as an internal ribosome entry site (IRES).

In some embodiments, mammalian expression vectors include, but are not limited to, pcDNA3, pcDNA3.1 (±), pGL3, pZeoSV2 (±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses can be used. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression of the peptide of the invention. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, the viral vectors that are produced are unable to spread laterally. In one embodiment, this characteristic can be useful if the desired purpose is to introduce a specified gene into only a localized number of targeted cells.

Various methods can be used to introduce an expression vector into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al., [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct can also include sequences engineered to optimize stability, production, purification, yield or activity of the expressed peptide.

In some embodiments, transformed cells are cultured under effective conditions, which allow for the expression of high amounts of a recombinant peptide. In some embodiments, effective culture conditions include, but are not limited to, effective media, bioreactor, temperature, pH and oxygen conditions that permit protein production. In one embodiment, an effective medium refers to any medium in which a cell is cultured to produce a recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, the cells can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant peptide of the invention either remains within the recombinant cell, secreted into the fermentation medium, secreted into a space between two cellular membranes, such as the periplasmic space in E. coli; or retained on the outer surface of a cell or viral membrane. In one embodiment, following a predetermined time in culture, recovery of the recombinant polypeptide is affected.

In one embodiment, the phrase “recovering the recombinant peptide” as used herein, refers to collecting the whole fermentation medium containing the peptide and need not imply additional steps of separation or purification.

In one embodiment, a peptide of the invention is purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode the polypeptide of the invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety, and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the peptide of the invention is retrieved in “substantially pure” form that allows for the effective use of the protein in the applications described herein.

As used herein, the term “substantially pure” describes a peptide/polypeptide or other material which has been separated from its native contaminants. Typically, a monomeric peptide is substantially pure when at least about 60 to 75% of a sample exhibits a single peptide backbone. Minor variants or chemical modifications typically share the same peptide sequence. A substantially pure peptide can comprise over about 85 to 90% of a peptide sample, and can be over 95% pure, over 97% pure, or over about 99% pure, or any value and range therebetween. Each possibility represents a separate embodiment of the invention. Purity can be measured on a polyacrylamide gel, with homogeneity determined by staining. Alternatively, for certain purposes high resolution may be necessary and HPLC or a similar means for purification can be used. For most purposes, a simple chromatography column or polyacrylamide gel can be used to determine purity.

The term “purified” does not require the material to be present in a form exhibiting absolute purity, exclusive of the presence of other compounds. Rather, it is a relative definition. A peptide is in the “purified” state after purification of the starting material or of the natural material by at least one order of magnitude, 2 or 3, or 4 or 5 orders of magnitude.

In one embodiment, the peptide of the invention is substantially free of naturally-associated host cell components. The term “substantially free of naturally-associated host cell components” describes a peptide or other material which is separated from the native contaminants which accompany it in its natural host cell state. Thus, a peptide which is chemically synthesized or synthesized in a cellular system different from the host cell from which it naturally originates will be free from its naturally-associated host cell components.

In one embodiment, the peptide of the invention can also be synthesized using in vitro expression systems. In one embodiment, in vitro synthesis methods are well known in the art and the components of the system are commercially available. Non-limited example for in vitro system includes but is not limited to in vitro translation.

### Pharmaceutical Composition

According to some embodiments, there is provided a composition comprising any one of: (a) the peptide of the invention; (b) the dimeric peptide of the invention; and (c) a combination of (a) and (b), and an acceptable carrier.

In some embodiments, the composition is a pharmaceutical composition. In some embodiments, the carrier is a pharmaceutical carrier.

In some embodiments, the pharmaceutical composition is for use in the treatment or prevention of a K63Ub-related disease, in a subject in need thereof. In some embodiments, a K63Ub-related disease comprises a disease related to dysregulation of Lys63 Ub.

The term “K63Ub-related disease” refers to any disease wherein Lys63 Ub is involved in, induces, enhances, propagates, or any combination thereof, the pathogenesis, pathophysiology, or both, of the disease.

In some embodiments, a subject afflicted with or at increased risk of developing a K63Ub-related disease is characterized by increased or elevated levels of Lys63 Ub, compared to a healthy subject. In some embodiments, a subject afflicted with or at increased risk of developing K63Ub-related disease is characterized by elevated levels of ubiquitin E3 ligases compared to a healthy subject. In some embodiments, a subject afflicted with or at increased risk of developing K63Ub-related disease is characterized by increased levels of ring finger protein 8 (RNF8), or RNF168, compared to a healthy subject. In some embodiments, a subject afflicted with or at increased risk of developing K63Ub-related disease is characterized by elevated levels of mutated DNA, compared to a healthy subject. In some embodiments, a subject afflicted with or at increased risk of developing K63Ub-related disease is characterized by increased levels, abundance, phosphorylation levels, or any combination thereof, of H2A histone family member X (H2AX), compared to a healthy subject.

In some embodiments, a K63-related disease comprises a cell proliferation-related disease. In some embodiments, a cell proliferation-related disease comprises cancer. In some embodiments cancer comprises adenocarcinoma. In some embodiments, cancer comprises squamous cell carcinoma. In some embodiments, cancer comprises sarcoma. In some embodiments, cancer comprises osteosarcoma.

The term “cell proliferation-related disease” refers to any disease in which dysregulation of cell proliferation is at least one mechanism involved in the disease pathogenesis.

In some embodiments, the pharmaceutical composition is characterized by having pro-apoptotic activity. In some embodiments, the pharmaceutical composition is characterized by cell cycle arrest activity.

As used herein, the term “pro-apoptotic activity” refers to a compound's ability to induce, increase, enhance, propagate, facilitate, contribute, being involved, or any combination thereof, with programed cell death.

As used herein, the term “cell cycle arrest” refers to slowing, halting, inhibiting, blocking, or any combination thereof, progression of a cell through the cell cycle. The cell can be induced to arrest at any point/phase/stage of a cell cycle. In some embodiments, cell cycle arrest activity comprises halting, inhibiting, blocking, or any combination thereof, progression of a cell through any one of: G0, G1, S, G2, or M.

In some embodiments, the pharmaceutical composition is for use in the treatment of cancer in a subject in need thereof.

In some embodiments, the pharmaceutical composition facilitates administration of a compound to an organism. According to another embodiment, the invention provides a pharmaceutical composition comprising as an active ingredient a therapeutically effective amount of the peptide of the invention, the dimeric peptide of the invention, or both.

In another embodiment, the pharmaceutical composition of the invention may be formulated in the form of a pharmaceutically acceptable salt of the peptides of the present invention or their analogs, or derivatives thereof. In another embodiment, pharmaceutically acceptable salts include those salts formed with free amino groups such as salts derived from non-toxic inorganic or organic acids such as hydrochloric, phosphoric, acetic, oxalic, tartaric acids, and the like, and those salts formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

As used herein, the term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the polypeptide of the invention, preferably in a substantially purified form, together with a suitable amount of carrier so as to provide the form for proper administration to the subject.

According to an embodiment of the invention, pharmaceutical compositions contain 0.1-95% of the peptide(s) of the invention, functional analog thereof, or derivatives thereof. According to another embodiment of the invention, pharmaceutical compositions contain 1-70% of the peptide(s). According to another embodiment of the invention, the composition or formulation to be administered may contain a quantity of peptide(s), according to embodiments of the invention in an amount effective to treat the condition or disease of the subject being treated.

An embodiment of the invention relates to a peptide, dimeric peptide, or both, of the invention, presented in unit dosage form and prepared by any of the methods well known in the art of pharmacy. In an embodiment of the invention, the unit dosage form is in the form of a tablet, capsule, lozenge, wafer, patch, ampoule, vial or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

According to one embodiment, the compositions of the invention are administered in the form of a pharmaceutical composition comprising at least one of the active components of this invention (e.g., peptide, such as, but not limited to a cyclic peptide) together with a pharmaceutically acceptable carrier or diluent. In another embodiment, the compositions of this invention can be administered either individually or together in any conventional oral, parenteral or transdermal dosage form. In some embodiments, the pharmaceutical composition further comprises at least one anticancer agent such as a chemotherapeutic agent. In some embodiments, the pharmaceutical composition is adopted for combined administration with an anti-cancer therapy such as chemotherapy, radiotherapy, immunotherapy, hormonal therapy, toxin therapy or surgery.

As used herein, the terms “administering”, “administration”, and like terms refer to any method which, in sound medical practice, delivers a composition containing an active agent to a subject in such a manner as to provide a therapeutic effect.

Depending on the location of the tissue of interest, the peptide of the invention can be administered in any manner suitable for the provision of the peptide to cells within the tissue of interest. Thus, for example, a composition containing the peptide of the invention can be introduced, for example, into the systemic circulation, which will distribute the peptide to the tissue of interest. Alternatively, a composition can be applied topically to the tissue of interest (e.g., injected, or pumped as a continuous infusion, or as a bolus within a tissue, applied to all or a portion of the surface of the skin, etc.).

In some embodiments, the pharmaceutical compositions comprising the peptide are administered via oral, rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, a peptide of the invention, derivative, functional analog thereof or a fragment thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

According to some embodiments, the peptide, dimeric peptide, or both, of the invention, can be delivered in a controlled release system. In another embodiment, an infusion pump can be used to administer a peptide such as the one that is used, for example, for delivering insulin or chemotherapy to specific organs or tumors. In another embodiment, the peptide, dimeric peptide, or both, of the invention is administered in combination with a biodegradable, biocompatible polymeric implant, which releases the peptide over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

The presently described peptide may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The compositions also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In one embodiment, it will be appreciated that the peptide, dimeric peptide, or both, of the invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the peptide is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the polypeptide is the amount of the polypeptide necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

Pharmaceutical compositions containing the presently described peptide(s) as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, compositions including the preparation of the present invention formulated in a compatible pharmaceutical carrier are prepared, placed in an appropriate container, and labeled for treatment of an indicated condition.

In one embodiment, compositions of the invention are presented in a pack or dispenser device, such as an FDA approved kit, which contains one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods of Treatment

According to some embodiments, there is provided a method for ameliorating or treating a subject afflicted with a K63Ub-related disease, the method comprising administering to the subject a therapeutically effective amount of any one of: the peptide of the invention; the dimeric peptide of the invention; the pharmaceutical composition of the invention, or any combination thereof, thereby ameliorating or treating the subject afflicted with K63Ub-related disease.

According to some embodiments, there is provided a method for ameliorating or treating a subject afflicted with a cell-proliferation related disease, the method comprising administering to the subject a therapeutically effective amount of any one of: the peptide of the invention; the dimeric peptide of the invention; the pharmaceutical composition of the invention, or any combination thereof, thereby ameliorating or treating the subject afflicted with cell-proliferation related disease.

According to some embodiments, there is provided a method for ameliorating or treating a subject afflicted with cancer, the method comprising administering to the subject a therapeutically effective amount of any one of: the peptide of the invention; the dimeric peptide of the invention; the pharmaceutical composition of the invention, or any combination thereof, thereby ameliorating or treating the subject afflicted with cancer. In some embodiments cancer is adenocarcinoma. In some embodiments, cancer is squamous cell carcinoma. In some embodiments, cancer is sarcoma. In some embodiments, cancer is osteosarcoma.

In some embodiments, there present invention is directed to a method for treating, ameliorating, reducing and/or preventing a condition associated with increased accumulation of K63 Ub in a cell of a subject in need thereof, the method comprising the step of: administering to the subject a pharmaceutical composition comprising a therapeutically effective amount of the peptide of the invention, the dimeric peptide of the invention, or both.

In some embodiments, there present invention is directed to a method for treating, ameliorating, reducing and/or preventing a condition associated with increased proliferation activity of a cell in a subject in need thereof, the method comprising the step of: administering to the subject a pharmaceutical composition comprising a therapeutically effective amount of the peptide of the invention; the dimeric peptide of the invention; or both.

In some embodiments, there present invention is directed to a method for treating, ameliorating, reducing and/or preventing a condition or a disease associated with increased apoptosis-resistance activity of a cell in a subject in need thereof, the method comprising administering to the subject a therapeutically effective amount of a pharmaceutical composition as disclosed herein.

In some embodiments, any one of a K63Ub-related disease is characterized by increased accumulation of K63Ub in a cell of a subject.

As used herein the terms “cancer” or “pre-malignancy” refer to diseases associated with cell proliferation. Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma, and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refer to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but are not limited to germinoma, dysgerminoma and seminoma. In one embodiment, non-germinomatous or non-seminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

As used herein, “cancer or pre-malignant cell proliferation” is a molecular process which further to increased cell proliferation rates requires increased deubiquitination activity.

According to some embodiments, there is provided a method for increasing an amount of fragmented DNA in a cell of a subject, comprising contacting the cell with an effective amount of any one of: the peptide of the invention; the dimeric peptide of the invention; or both.

According to some embodiments, there is provided a method for reducing abundance, levels, or both, of Lys63 Ub in a cell, the method comprising contacting the cell with an effective amount of any one of: the peptide of the invention; the dimeric peptide of the invention; and a composition comprising any one of same.

According to some embodiments, there is provided a method for inducing or increasing apoptosis rate of a cell, comprising contacting the cell with an effective amount of any one of: the peptide of the invention; the dimeric peptide of the invention; and a composition comprising any one of same.

According to some embodiments, there is provided a method for inducing a cell cycle arrest in a cell, the method comprising contacting a cell with an effective amount of any one of: the peptide of the invention; the dimeric peptide of the invention; and a composition any one of same.

In some embodiments, the cell is a cancer or cancerous cell. In some embodiments, the cell is a cancer cell or a cancerous cell of a subject. In some embodiments, the cell is obtained or derived from a subject.

In some embodiments, the method comprises reducing deubiquitination activity or rates, abundance or levels of deubiquitinated protein(s), reduced proteasomal activity or proteasomal protein degradation rate, or any combination thereof, in a cell. In some embodiments, the cell is a cancer or cancerous cell. In some embodiments, the cell is a cell of a subject. In some embodiments, the cell is obtained or derived from a subject.

In some embodiments, the treating or ameliorating comprises: increasing DNA fragmentation rate, increasing the numbers of mutations, such as, but not limited to deleterious mutations, increasing the accumulation rate, levels, or both, of fragmented DNA, or any combination thereof, in a cancer or cancerous cell in a subject. In some embodiments, the treating comprises reducing drug resistance of a cancer or a cancerous cell in a subject. In some embodiments, a cancer or cancerous cell is characterized by increased deubiquitination activity compared to a non-cancerous cell or a benign cell. In another embodiment, the treating comprises reducing deubiquitination activity in a cancer or cancerous cell in a subject. In some embodiments, the treating comprises reducing viability or survival a cancer or cancerous cell in a subject. In some embodiments, reducing deubiquitination activity comprises increasing apoptosis rates in or of a cancer or cancerous cell in a subject.

In some embodiments, the terms “reduce” or “reducing” comprise at least 10%, at least 20%, at least 30%, at least 40%, at least 50%, at least 60%, at least 70%, at least 80%, at least 90%, or 100% reduction, or any value and range therebetween. Each possibility represents a separate embodiment of the invention. In some embodiments, reducing is 1-5%, 4-10%, 8-20%, 15-30%, 25-40%, 35-55%, 50-70%, 60-80%, 75-90%, 90-99%, or 95-100% reduction. Each possibility represents a separate embodiment of the invention. In some embodiments, reducing is at least 2-fold, at least 3-fold, at least 5-fold, at least 10-fold, at least 15-fold, at least 20-fold, at least 40-fold, at least 75-fold, at least 100-fold, at least 150-fold, at least 200-fold, at least 500-fold, or at least 1,000-fold reduction, or any value and range therebetween. Each possibility represents a separate embodiment of the invention.

The terms “inhibiting”, “reducing” and “decreasing” are interchangeable.

In some embodiments, the term “increase” or “increasing” used in the abovementioned embodiments (such as for pro-apoptotic activity, cell apoptosis rate, or others), is by at least 10%, by at least 20%, by at least 30%, by at least 40%, by at least 50%, by at least 60%, by at least 70%, by at least 80%, by at least 90%, or by at least 100% compared to control, or any value and range therebetween. Each possibility represents a separate embodiment of the invention. In some embodiments, increasing is by 1-5%, 4-10%, 8-20%, 15-30%, 25-40%, 35-55%, 50-70%, 60-80%, 75-90%, 90-99%, or 95-100% compared to control. Each possibility represents a separate embodiment of the invention. In some embodiments, increasing is by at least 2-fold, by at least 3-fold, by at least 5-fold, by at least 10-fold, by at least 15-fold, by at least 20-fold, by at least 40-fold, by at least 75-fold, by at least 100-fold, by at least 150-fold, by at least 200-fold, by at least 500-fold, or by at least 1,000-fold compared to control, or any value and range therebetween. Each possibility represents a separate embodiment of the invention.

In some embodiments, ubiquitination/deubiquitination kinetics or dynamics, are detected by any assay known to a person of ordinary skill in the art, including immune-assays, western-blot, immune-histochemistry, and the like, such as for detecting Lys63 Ub. In some embodiments, protein degradation and proteasomal activity are detected by any acceptable method, including immune-assays, western-blot, immune-histochemistry, pulse-chase assay, and the like, all of which are well known to one of ordinary skill in the art.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with and/or at risk of being afflicted with a condition associated with increased cell proliferation, deubiquitination activity, or combination thereof.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

In some embodiments, the method further comprising administering to the subject any one of: the peptide of the invention; the dimeric peptide of the invention; and the pharmaceutical composition of the invention, in conjunction with a therapeutically effective amount of anti-cancer therapy, including, but not limited to: a surgical therapy, chemotherapy, radiation therapy, cryotherapy, hormone therapy, immunotherapy, cytokine therapy, or any combination thereof.

In some embodiments, in conjunction comprises a single pharmaceutical composition comprising the peptide of the invention; the dimeric peptide of the invention; and the pharmaceutical composition of the invention, and the anti-cancer therapy. In some embodiments, in conjunction comprises separate pharmaceutical compositions, a first comprising the peptide of the invention; the dimeric peptide of the invention; and the pharmaceutical composition of the invention, and a second comprising the anti-cancer therapy.

In some embodiments, first and second pharmaceutical compositions as described herein. Are provided concomitantly or separately.

As used herein, the term “high binding affinity” refers to binding with a dissociation constant in the nanomolar scale. In some embodiments, high binding affinity is with a KD ranging from 0.1 nM to 10 nM, 1 nM to 100 nM, 50 nM to 250 nM, 15 nM to 1,500 nM, or 20 nM to 300 nM. Each possibility represents a separate embodiment of the invention.

### General

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Peptides are synthesized by solid-phase peptide synthesis (SPPS) approach using an automated peptide synthesizer (CS336X, CSBIO) or manually in teflon filter equipped syringes, purchased from Torviq. All used chemicals are analytical grade unless specified. Palladium (II) chloride (PdCl2), 4-bromomethyl-7-methoxycoumarin, Dimethyl sulfoxide (DMSO), HEPES (4-(2-Hydroxyethyl) piperazine-1-ethanesulfonic acid, Iodoacetamide, Decafluorobiphenyl (Reagent grade), Hexafluorobenzene (Reagent grade), Benzyl bromide (Reagent grade) were purchased from Sigma-Aldrich. Trifluoroacetic acid (TFA), dichloromethane (DCM), Diisopropylethylamine (DIEA), and N, N-dimethylformamide (DMF) were purchased from Biolab. Tetramethylrhodamine-5-maleimide (TAMRA) and Fluorescein-5-Maleimide (FITC) were purchased from Thermo Fisher Scientific. 3-(chloromethyl)benzoic acid,2-Chloroacetic acid were purchased from Acros Organics. Tert-Butyloxycarbonyl (Boc), 9-fluorenylmethoxycarbonyl (Fmoc) protected amino acids were purchased from GL Biochem. Resins were purchased from CreoSalus. Dithiothreitol (DTT) and triisopropylsilane (TIPS) were purchased from Alfa Aesar. All coupling reagents [(6-chlorobenzotriazol-1-yl)oxy-(dimethylamino)methylidene]-dimethylazanium hexafluorophosphate (HCTU),1-[Bis(dimethylamino)methylene]-1H-1,2,3-triazolo[4,5-b]pyridinium 3-oxid hexafluorophosphate (HATU)] and hydroxybenzotriazole (HOBt) were purchased from GL Biochem and Luxembourg Bio Technologies. A Thermo instrument (Dionex Ultimate 3000) using Xbridge (4.6×150 mm, 3.5 μm, BEH300 C4, waters) column was used for analytical high-performance liquid chromatography (HPLC) with 1.2 ml/min flow rate. Thermo Scientific instrument (Dionex Ultimate 3000) used Jupiter C4 (250×10 mm, 10 μm, 300 Å, Phenomenex) for semi-preparative hulk with 4.0 ml/min flow rate. Thermo Scientific instrument (Dionex Ultimate 3000) used Jupiter C4 (250×22.4 mm, 10 μm, 300 Å, Phenomenex) for preparative HPLC with 15.0 ml/min flow rate. All the peptides were purified by HPLC and characterized by mass spectrometry.

Dulbecco's modified eagle's medium (DMEM), Fetal bovine serum (FBS), L-Glu, antibiotics, (penicillin/streptomycin), trypsin/EDTA and phosphate-buffered saline (PBS) were purchased from biological industries. Trans-blot turbo (0.2 μm PVDF) membrane for blotting and electrophoresis set-up were purchased from Bio-Rad. Streptavidin Agarose resin, Hoechst 33342 solution (20 mM), imperial blue strain, cell culture plates, and high-capacity streptavidin agarose resin were purchased from Thermo-fisher. A non-protein Instant Block buffer for western blotting application was purchased from Gene Bio-Application L.T.D. Immobilon Crescendo Western HRP substrate was purchased from Millipore. The comet assay kit was purchased from Abcam. MEBCYTO Apoptosis kit or Annexin V-FITC kit was purchased from medical and biological laboratories co., LTD., μ-Slide 8 well for live-cell confocal microscopy was purchased from ibidi and poly-lysine hydrobromide was purchased from Sigma. Recombinant monoclonal gamma H2A.X (phospho S139) antibody, rabbit monoclonal Histone H2A.X [EPR895], recombinant monoclonal Ubiquitin (linkage-specific Lys63) antibody, Ubiquitin (linkage-specific Lys48) antibody, the secondary goat anti-rabbit or anti-mouse IgG (HRP) antibody were purchased from Abcam, recombinant monoclonal FLAG (R) M2 antibody was purchased from Sigma, and recombinant mouse monoclonal Ubiquitin (P4D1) antibody was purchased from Santa Cruz Biotechnology.

### Synthesis of Biotinylated-Lys63 Linked Di-Ub

For the synthesis of the biotinylated-Lys63 linked Di-Ub chain, we prepared Ub building blocks 1 and 2 (biotin-Ub-MMP) using standard Fmoc-solid-phase peptide synthesis (Fmoc-SPPS) with the previously mentioned modifications (Kumar K S A et al., 2010). The building blocks 1 and 2 are ligated to give 3. Then 3 is subjected to radical-mediated desulfurization following HPLC (employing C4 column and gradient flow of 0-60% B) and FPLC purification steps gave the desired native Lys63-linked Di-Ub chain 4 with high purity. Other Ub chains such as Lys11-linked Di-Ub, Lys29-linked Di-Ub, Lys48-linked Di-Ub, Linear Di-Ub, and Lys48-linked Tetra-Ub were prepared as described previously (Kumar K S A et al., 2011).

### RaPID Method for the Identification of Cyclic Peptide 1

For aminoacylated tRNA synthesis, the initiator tRNA was charged with mCIBz using enhanced flexible ribosome (eFX), and this is termed as tRNAfMetCAU. Reaction conditions for the preparation of tRNA solution were employed as in (Suga H et al., 2020). Next, a microhelix RNA charging experiment was performed as previously described (Suga H et al., 2020). For flexible in vitro translation (FIT), the components of the translation system were employed as in (Jongkees S A K et al., 2017) were used.

RaPID selection was performed as described in (Nawatha M at al., 2019), using the buffer selection=1×TBS-T (50 mM Tris, 150 mM NaCl, 0.1% Tween 20, pH 7.60) and 33 μM mCIBz-tRNAfMetCAU. Positive and negative readouts were collected through clone assay, where clones with P/N>20 were chosen as candidate peptides.

PCR amplification was performed following the extraction of the resulting sequence and categorized based on Python script, which gives motif corresponding to NNK library region with the correct length.

### Screening of Peptides Using a Fluorescence-Based Competitive Assay

Streptavidin-coated microplate (96 well plate) was washed with HEPES buffer (50 Mm HEPES, 150 Mm NaCl, pH 7.30) and then in each well incubated 1 μg of biotinylated-Lys63-linked Di-Ub in 100 μl of the same buffer at RT. for 30 min. Additional wells were kept without biotinylated-Lys63-Di-Ub for blank subtraction. After the washing step, peptide standard (CP1) and unlabeled peptides were incubated (5.0 molar equivalent relative to biotin-Lys63-linked Di-Ub) at RT for 30 minutes for saturation binding to the target. Then 1 molar equivalent of FITC labeled peptide-standard (CP1-FITC) relative to biotinylated-Lys63-linked Di-Ub was incubated to compete with unlabeled peptides at RT for 30 min. After releasing bounded peptides on treatment with 6 M Guanidinium chloride (Gnd·HCl), the fluorescence values were measured (λex=480 nm and λem=525 nm). These values were normalized and then calculated the relative change in binding with the following formula:

Y=[1−(a/b)]×100
Where Y=Signal change relative to the standard (CP1) (in terms of %).
a=Measured signal value of each peptide candidate (in Relative Fluorescence Units).
b=Measured signal value of standard peptide (CP1).
   The dissociation constant (KD) of fluorescein-labeled peptide (CP1-FITC) was calculated in accordance with the literature procedure (Vamisetti G B et al., 2021).

### Synthesis of Cyclic Peptide 1

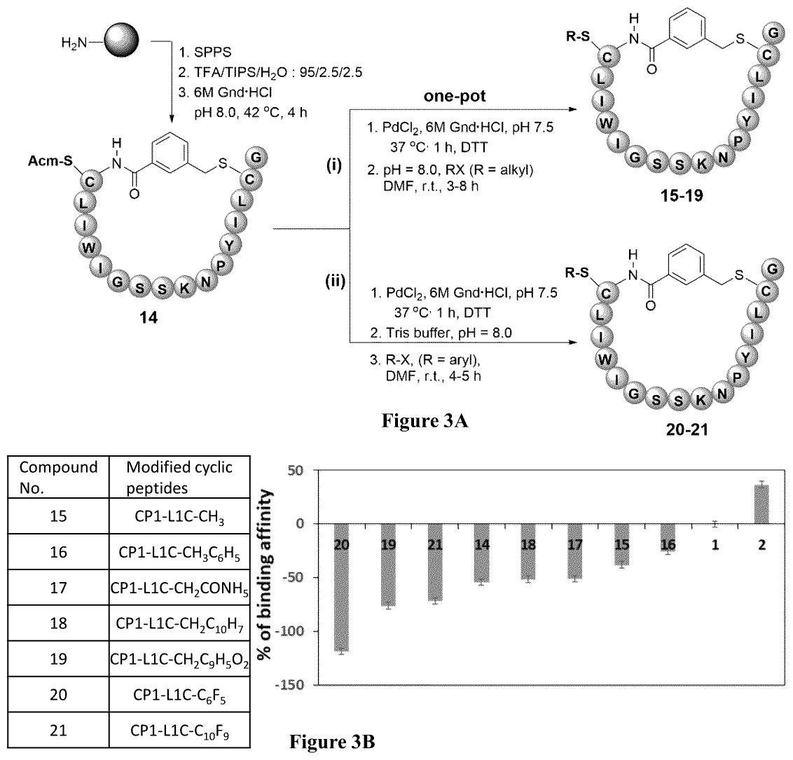

Fmoc-SPPS was performed on a Rink amide resin (0.26 mmol/g, 0.1 mmol scale) for synthesizing peptide 1 as shown in FIG. 3A. The Peptide was synthesized using amino acids (4.0 equiv.), HCTU (4.0 equiv.), and DIEA (8.0 equiv.) at room temperature. Fmoc protecting group was removed by treating the resin with 20% piperidine in DMF containing 0.1 mmol HOBt (3:5:3 min). To the N-terminal of the sequence, 3-(chloromethy)-benzoic acid was coupled as per the literature procedure.4 The peptide was cleaved from the resin using the cocktail TFA/H₂O/TIS (95:2.5:2.5) and then precipitation in cold diethyl ether and lyophilization. The cyclization was performed by dissolving crude peptide in 6 M Gnd. HCl and adjusted to pH 8.0 with NaOH followed by incubation at 42° C. for 4 h. Then crude peptide was purified by using HPLC with a C₄ column by using a gradient flow of 0-60% B in 60 min.

### Synthesis of Cyclic Peptides, 2-14

Fmoc-SPPS was applied for synthesizing the cysteine mutated cyclic peptides. An orthogonally protected Cys (Acm) was incorporated at various positions of the 1 (CP1), for the synthesis of different cyclic peptides with Cys mutation. After cleavage from resin and cyclization, purification was performed. For Acm removal, 5 the peptide CP1-S8C-Acm (10.0 mg, 5.04×10-3 mmol, 1.0 equiv.) was dissolved in 6 M Gnd·HCl/200 mM phosphate buffer (pH 7.50, 2524.8 μl, 2 mM). Then, PdCl2 (8.94 mg, 10.0 equiv.) was dissolved in 100 μl of 6 M Gnd·HCl/200 mM phosphate buffer (pH 7.50) at 37° C. for 10 min and it was added to the peptide solution. The reaction mixture was incubated at 37° C. for 1 h. Next, the reaction mixture was quenched with the dithiothreitol, DTT, (40.0 equiv., 2.05×10⁻¹ mmol). After centrifugation, the supernatant was injected into HPLC using a semi-preparative C4 column with a gradient flow of 0-60% B in 60 min to give free thiol-containing cyclic peptide 9, CP1-L1C (3.85 mg, 40% yield). A similar procedure was applied for synthesizing other cyclic peptides 2-13 and cyclic peptide 14.

The prepared benzyl thioether-linked cyclic peptides are presented in the following table:

TABLE 1
Representing the prepared benzyl thioether-linked cyclic peptides 1-14

| | | Observed | Calculated |
|---|---|---|---|
| | | mass | mass |
| | | [M + H]+ | [M + H]+ |
| S. No. | Cysteine mutated cyclic peptide | (Da) | (Da) |
| 1 | 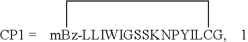 | 1891.5 ± 0.2 | 1891.3 |
| 2 | 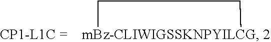 | 1881.5 ± 0.2 | 1881.3 |
| 3 | 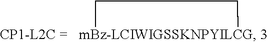 | 1881.5 ± 0.1 | 1881.3 |
| 4 | 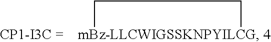 | 1881.5 ± 0.2 | 1881.3 |
| 5 | 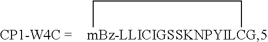 | 1808.5 ± 0.2 | 1808.2 |
| 6 | 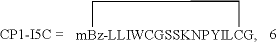 | 1881.5 ± 0.2 | 1881.3 |
| 7 | 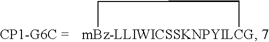 | 1937.5 ± 0.1 | 1937.4 |
| 8 | 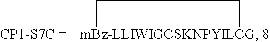 | 1907.5 ± 0.2 | 1907.3 |
| 9 | 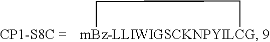 | 1907.5 ± 0.1 | 1907.3 |
| 10 | 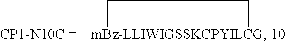 | 1880.5 ± 0.2 | 1880.3 |
| 11 | 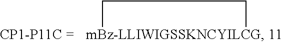 | 1897.5 ± 0.1 | 1897.3 |
| 12 | 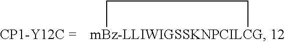 | 1831.2 ± 0.1 | 1831.2 |
| 13 | 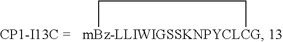 | 1881.4 ± 0.1 | 1881.3 |
| 14 | 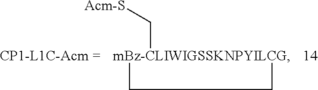 | 1952.1 ± 0.1 | 1952.3 |

### Synthesis of Cysteine-Modified Derivatives of Cyclic Peptide 2

The cyclic peptide 2, CP1-L1C, was further modified with alkylated and arylating reagents for preparing different Cys-modified cyclic peptides as described below:

### (a) Synthesis of Cysteine-Alkylation Derivatives, 15-19

Various cysteine-alkylated derivatives from cyclic peptide 14, CP1-L1C-Acm, were prepared in one pot as previously described (Vamisetti G B et al., 2021). The synthesis of each derivative was performed as follows.

### (i) Synthesis of Cyclic Peptide 15, CP1-L1C-CH3

Acm removal for peptide 14, CP1-L1C-Acm (10.0 mg, 5.11×10⁻³ mmol, 1.0 equiv.) was performed. After quenching and centrifugation steps of the reaction mixture, separate the supernatant and adjust pH to 8.0 with NaOH. Dissolve 500 equiv. of iodomethane (CH₃₁) in 500 μl of DMF and was added to the reaction mixture at room temperature. The progress of the reaction mixture was monitored by HPLC using a C₄ analytical column with a gradient flow of 0-60% B in 30 min. The reaction was completed within 3 h. Then the reaction mixture was purified using a preparative C₄ column with a gradient flow of 0-60% in 60 min to give 15, CP1-L1C-CH3 (4.27 mg, 44% yield).

### (ii) Synthesis of Cyclic Peptide 16, CP1-L1C-CH2C6H5

Next to Acm removal of peptide 14 (10.0 mg, 5.11×10⁻³ mmol, 1.0 equiv.), the aqueous portion was separated and adjusted its pH to 8.0 with NaOH. Subsequently, 500 equiv. benzyl bromide (C6H5CH2Br) dissolved in 500 μl of DMF was added to the reaction mixture at room temperature. The progress of the reaction mixture was monitored by HPLC using a C4 analytical column with a gradient flow of 0-60% B in 30 min. The reaction was completed in 6 h and was purified using a preparative C4 column with a gradient flow of 0-60% in 60 min to give 16, CP1-L1C-CH2C6H5 (4.04 mg, 40% yield).

(iii) Synthesis of Cyclic Peptide 17, CP1-L1C-CH2CONH2

Following the Acm removal of peptide 14 (10.0 mg, 5.11×10⁻³ mmol, 1.0 equiv.), the aqueous portion was separated and adjusted its pH to 8.0 with NaOH. Next, 500 equiv. of 2-iodoacetamide (ICH2CONH2) dissolved in 500 μl of DMF was added to the reaction mixture at room temperature. The progress of the reaction mixture was monitored by HPLC using a C4 analytical column with a gradient flow of 0-60% B in 30 min. The reaction was completed in 5 h and was purified using a preparative C4 column with a gradient flow of 0-60% in 60 min to give 17, CP1-L1C-CH2CONH2 (4.76 mg, 49% yield).

### (iv) Synthesis of Cyclic Peptide 18, CP1-L1C-CH2C10H7

After Acm removal of peptide 14 (10.0 mg, 5.11×10⁻³ mmol, 1.0 equiv.), the aqueous portion was separated and adjusted its pH to 8.0 with NaOH. Then, 500 equiv. of 2-(bromomethyl) naphthalene (C10H7CH2Br) dissolved in 500 μl of DMF was added to the reaction mixture at room temperature. The progress of the reaction mixture was monitored by HPLC using a C4 analytical column with a gradient flow of 0-60% B in 30 min. The reaction was completed in 7 h and was purified using a preparative C4 column with a gradient flow of 0-60% in 60 min to give 18, CP1-L1C-CH2C10H7 (4.14 mg, 40% yield).

### (v) Synthesis of Cyclic Peptide 19, CP1-L1C-CH2C10H7O3

After Acm removal of 14 (10.0 mg, 5.11×10⁻³ mmol, 1.0 equiv.), the aqueous portion was separated and adjusted its pH to 8.0 with NaOH. After quenching and centrifugation steps of the reaction mixture, separate the supernatant and adjust pH to 8.0 with NaOH. Subsequently, 500 equiv. of 4-bromomethyl-7-methoxycoumarin (BrCH2C10H7O3) dissolved in 500 μl of DMF was added to the reaction mixture at room temperature. The progress of the reaction mixture was monitored by HPLC using a C4 analytical column with a gradient flow of 0-60% B in 30 min. The reaction was completed in 8 h and was purified using a preparative C4 column with a gradient flow of 0-60% in 60 min to give 19, CP1-L1C-CH2C10H7O3 (4.23 mg, 40% yield).

### (b) Synthesis of Cysteine-Arylation Derivatives, 20-21

### (vi) Synthesis of Cyclic Peptide 20, CP1-L1C-C6F5

Peptide 2, CP1-L1C (10.0 mg, 5.31×10⁻³ mmol, 1.0 equiv.) was dissolved in 2.7 mL of 50 mM Tris base in DMF. Then a solution of hexafluorobenzene (6.15 μl, 10.0 equiv.) in 100 μl of DMF was added to the peptide solution. Then the reaction mixture was vigorously mixed for 30 seconds and kept at room temperature. The progress of the reaction mixture was monitored by HPLC using a C4 analytical column with a gradient flow of 0-60% B in 30 min. The reaction was completed in 4 h and was purified using a preparative C4 column with a gradient flow of 0-60% in 60 min to give 20, CP1-L1C-C6F5 (4.03 mg, 37% yield).

(vii) Synthesis of Cyclic Peptide 21, CP1-L1C-C10F9

Peptide 2, CP1-L1C (10.0 mg, 5.31×10⁻³ mmol, 1.0 equiv.) was dissolved in 2.7 mL of 50 mM Tris base in DMF. Then a solution of decafluorobiphenyl (5.32 mg, 3.0 equiv.) in 100 μl of DMF was added to the peptide solution. Then the reaction mixture was vigorously mixed for 30 seconds and kept at room temperature. The progress of the reaction mixture was monitored by HPLC using a C4 analytical column with a gradient flow of 0-60% B in 30 min. The reaction was completed in 5 h and was purified using a preparative C4 column with a gradient flow of 0-60% in 60 min to give 21, CP1-L1C-C10F9 (4.08 mg, 35% yield).

### Synthesis of CP1-FITC and CP1-TAMRA

For synthesizing FITC labeled cyclic peptide (CP1-FITC), Fmoc-Cys (Acm) was coupled as the first amino acid for a late-stage modification. After cyclization and Acm removal steps, the peptide with free thiol functional group was dissolved in 6 M Gnd·HCl/200 mM phosphate buffer (pH 7.50, 2 mM). Then fluorescein-5-maleimide (2.0 equiv.)/tetramethylrhodamie-5-maleimide (TAMRA, 1.50 equiv.) dissolved in DMF was added to peptide solution and kept at room temperature under dark conditions. The reaction progress was monitored by HPLC using a C4 analytical column with a gradient flow of 0-60% B in 30 min. The reaction was completed in 2 h and was filtered and purified by injecting into the HPLC using a C4 semi prep-column with a gradient flow of 0-60% in 60 minutes.

### Synthesis of Cyclic Peptide 23

A Fmoc-SPPS procedure was applied for synthesizing peptide 22. All amino acids were coupled on an automated peptide synthesizer. For synthesizing TAMRA-labeled peptide, cysteine at 1 & 18 positions is orthogonally protected with Acm and -StBu respectively for later stage modifications. After coupling 3-(chloromethyl) benzoic acid at N-terminus, the resin was washed with DMF, MeOH, DCM and dried under vacuum. The peptide was cleaved from the resin using the cocktail TFA/H2O/TIS (95:2.5:2.5) and then precipitation in cold diethyl ether and lyophilization to give 22. The cyclization was performed by dissolving crude peptide 22 (4.0 mM) in 6 M Gnd·HCl/200 mM phosphate buffer and adjusted to pH 8.0 with NaOH followed by incubation at 42° C. The reaction progress was monitored by HPLC using C4 analytical column with a gradient flow of 0-60% B in 30 min, showing reaction completion within 4 h and was purified by HPLC using preparative column C4 with a gradient flow of 0-60% in 60 minutes to give peptide 23 in 43% yield.

### Synthesis of Cyclic Peptide 24

Peptide 23 (10.0 mg, 4.52×10⁻³ mmol, 1.0 equiv.) was dissolved in 6 M Gnd. HCl/200 mM phosphate buffer (pH 7.50, 2,258 μl, 2 mM). To this, a solution of TCEP (50.0 equiv.) dissolved in H2O was added following adjusted pH to 2.50 and incubated at 37° C.7 The reaction progress was monitored by HPLC using a C4 analytical column with a gradient flow of 0-60% B in 30 min. The reaction was completed within 4 h and was purified by HPLC using semi-preparative column C4 with a gradient flow of 0-60% in 60 minutes to give peptide 24 (3.84 mg, 40% yield).

### Synthesis of Cyclic Peptide 25

Peptide 24 (10.0 mg, 4.70×10⁻³ mmol, 1.0 equiv.) was dissolved in 6 M Gnd. HCl/200 mM phosphate buffer (pH 7.50, 2,351 μl, 2 mM). To this, add tetramethylrhodamine-5-maleimide (3.40 mg, 1.50 equiv.) dissolved in 50 μl of DMF at room temperature under dark conditions. The reaction progress was monitored by HPLC using a C4 analytical column with a gradient flow of 0-60% B in 30 min. The reaction was completed in 2 h and was purified by HPLC using semi-preparative column C4 with a gradient flow of 0-60% in 60 minutes to give peptide 25 (5.15 mg, 42% yield).

### Synthesis of Cyclic Peptide 2-TAMRA, 26

Peptide 25 (10.0 mg, 3.84×10⁻³ mmol, 1.0 equiv.) was dissolved in 6 M Gnd. HCl/200 mM phosphate buffer (pH 7.50, ˜1,918 μl, 2 mM). Then, PdCl2 (˜1.36 mg, 2.0 equiv.) was dissolved in 50 μl of 6 M Gnd. HCl/200 mM phosphate buffer (pH 7.50) at 37° C. for 10 min and it was added to the peptide solution following pH adjustment to 1.50 and the reaction was kept at room temperature. The reaction progress was monitored by HPLC using a C4 analytical column with a gradient flow of 0-60% B in 30 min showing reaction completion in 30 minutes. Then the reaction mixture was quenched by adding dithiothreitol, DTT, (8.0 equiv., 3.07×10⁻² mmol) followed by centrifugation and injection of the supernatant into HPLC using a semi-preparative C4 column with a gradient flow of 0-60% B in 60 min to give peptide 26 (1.85 mg, 19% yield) with free thiol.

### Synthesis of Cyclic Peptide 27

Peptide 26 (10.0 mg, 3.94×10⁻³ mmol, 1.0 equiv.) was dissolved in ˜ 2 ml of 50 mM Tris base in DMF. Then a solution of hexafluorobenzene (6.15 μl, 10.0 equiv.) in 100 μl of DMF was added to the peptide solution.6 The reaction mixture was vigorously mixed for 30 seconds and kept at room temperature. The progress of the reaction mixture was monitored by HPLC using a C4 analytical column with a gradient flow of 0-60% B in 30 min, showing reaction completion within 4 h. Then the reaction mixture was purified using a preparative C4 column with a gradient flow of 0-60% in 60 min to give 27 (3.30 mg, 31% yield).

Selectivity of Peptides 1 and 2 Against Ub Chains with Different Linkage

The binding of cyclic peptide 1 against Lys11 and Lys29 linked Di-Ub was examined using surface plasmon resonance (SPR) (FIG. 24, i & ii). The SPR analysis was performed according to the reported protocol. The relative binding affinity of cyclic peptides 1 and 2 for linear Di-Ub as well as Lys48 linked Di-Ub and Lys48 linked Tetra-Ub was tested applying the inventors fluorescence-based assay as shown in (iii), (iv), and (v) respectively (FIG. 24).

### Synthesis of FITC Labeled CP1-L1C-Acm Cyclic Peptide 28

A similar procedure mentioned in section 10 was applied to peptide 24 (10.0 mg, 4.70×10⁻³ mmol, 1.0 equiv.). 24 was dissolved in 6 M Gnd·HCl/200 mM phosphate buffer (pH 7.50, 2,351 μl, 2 mM) followed by the addition of fluoresceine-5-maleimide (6.02 mg, 3.0 equiv.) dissolved in 100 μl of DMF. The reaction was kept at room temperature in dark. The reaction progress was monitored by HPLC using a C4 analytical column with a gradient flow of 0-60% B in 30 min. The reaction was completed within 2 h and was purified by HPLC using semi-preparative column C4 with a gradient flow of 0-60% in 60 minutes to give peptide 28 in 44% isolated yield (5.28 mg).

### Synthesis of Cyclic Peptide 2-FITC, 29

A similar procedure to the synthesis of cyclic peptide 25 was applied to peptide 28 (10.0 mg, 3.92×10⁻³ mmol, 1.0 equiv.). 28 was dissolved in 6 M Gnd. HCl/200 mM phosphate buffer (pH 7.50, 1,958 μl, 2 mM). Then, PdCl2 (1.39 mg, 2.0 equiv.) was dissolved in 50 μl of 6 M Gnd. HCl/200 mM phosphate buffer (pH 7.50) at 37° C. for 10 min and it was added to the peptide solution following adjusted to pH 1.50 and kept the reaction at room temperature. After completion of the reaction, 30 mins, the reaction mixture was quenched with DTT following centrifugation and injected supernatant into HPLC using a semi-preparative C4 column with a gradient flow of 0-60% B in 60 min to give peptide 29 (2.04 mg, 21% yield) with free thiol.

### Determination of KD

### (i) Determination of KD of 1 Using SPR

The binding of cyclic peptide 1 (CP1) against the target, Lys63 linked Di-Ub was determined using BIACORE T100 instrument (GE Healthcare) equipped with a biotin CAPture kit series S chip. 50 mM HEPES (pH=7.30, 150 mM NaCl, 0.05% Tween 20, 2.0 mM DTT, 0.2% DMSO) was used as buffer. Biotinylated-Lys63 linked Di-Ub was loaded on the SPR chip. By using various concentrations of peptide.

### (ii) Determination of KD Value of CP1-FITC

As previously described (Vamisetti G B et al., 2021), the KD value of FITC-labeled cyclic peptide 1 (CP1-FITC) was determined. The dissociation constant KD was calculated as 95.8±2.3 nM.

(iii) Determination of KD Value of CP1-TAMRA

The fluorescence values were measured (λex=510 nm and λem=565 nm) similarly to CP1-FITC. The dissociation constant (KD) value of TAMRA-labeled CP1 (CP1-TAMRA) was calculated as 101.9±3.6 nM. All measurements were performed in triplicates.

### (iv) Determination of KD Value of 2-FITC, 29

The dissociation constant (KD) value of FITC-labeled cyclic peptide 29 was calculated in a similar way to CP1-FITC and was found as 43.2±4 nM.

### (v) Determination of KD Value of 2-TAMRA, 26

The fluorescence values were measured (λex=510 nm and λem=565 nm) in a similar way as described for CP1-FOTC. The dissociation constant (KD) value of TAMRA-labeled 26 (2-TAMRA) was calculated as 47.4±5.9 nM. All measurements were performed in triplicates.

### Synthesis of a Biotinylated Labeled Cyclic Peptide, 31

Peptide 24 (10.0 mg, 4.70×10⁻³ mmol, 1.0 equiv.) was dissolved in 6 M Gnd·HCl/200 mM phosphate buffer (pH 7.50, 2,351 μl, 2 mM). Then, biotinylated-PEG6-maleimide (6.60 mg, 9.40×10⁻³ mmol, 2.0 equiv.) was dissolved in 50 μl of DMF and was added to the peptide solution at room temperature. The reaction progress was monitored by HPLC using a C4 analytical column with a gradient flow of 0-60% B in 30 min. The reaction was completed in 45 min and was purified by HPLC using semi-preparative column C4 with a gradient flow of 0-60% in 60 minutes to give peptide 30 (9.04 mg, 68% yield). Next, Cys (Acm) deprotection step was performed according to the mentioned procedure for the synthesis of cyclic peptide 2-TAMRA, 26. The reaction progress was monitored by HPLC using a C4 analytical column with a gradient flow of 0-60% B in 30 min. The reaction was completed within 30 min, quenched with DTT. Following centrifugation, the mixture was injected into HPLC using a semi-preparative C4 column with a gradient flow of 0-60% B in 60 min to give peptide 31 in 22% isolated yield, (1.94 mg).

### Synthesis and Selectivity of a Scrambled Cyclic Peptide, 33

Fmoc-SPPS was applied for synthesizing the scrambled cyclic peptide 33 of cyclic peptide 2, similarly as described in section 6. After cleavage from resin and cyclization, purification was performed. For Acm removal,5 the peptide 32 (10.0 mg, 5.12×10⁻³ mmol, 1.0 equiv.) was dissolved in 6 M Gnd. HCl/200 mM phosphate buffer (pH 7.50, 2561.5 μl, 2 mM). Then, PdCl2 (9.07 mg, 10.0 equiv.) was dissolved in 100 μl of 6 M Gnd. HCl/200 mM phosphate buffer (pH 7.50) at 37° C. for 10 min and it was added to the peptide solution. The reaction mixture was incubated at 37° C. for 1 h. Next, the reaction mixture was quenched with the dithiothreitol, DTT, (40.0 equiv., 2.05×10⁻¹ mmol). After centrifugation, the supernatant was injected into HPLC using a semi-preparative C4 column with a gradient flow of 0-60% B in 60 min to give free thiol-containing cyclic peptide 33 (4.05 mg, 42% yield). Next, the relative binding affinity of cyclic peptide 33 for Lys63 linked Di-Ub was compared with specific Lys48 linked Di-Ub binder, mJ08-L8W in a similar process described for cyclic peptides 1 and 2.

### Synthesis of a Biotinylated Labeled Scrambled Cyclic Peptide, 38

A similar Fmoc-SPPS procedure described in section 4 was applied for synthesizing peptide 34. For synthesizing biotinylated peptides, cysteine at 1 & 18 positions is orthogonally protected with Acm and -StBu respectively for later stage modifications. After coupling 3-(chloromethyl)benzoic acid at N-terminus, the resin was washed with DMF, MeOH, DCM and dried under vacuum. The peptide was cleaved from the resin using the cocktail TFA/H₂O/TIS (95:2.5:2.5) and then precipitation in cold diethyl ether and lyophilization to give 34. The cyclization was performed by dissolving crude peptide 34 (4.0 mM) in 6 M Gnd. HCl/200 mM phosphate buffer and adjusted to pH 8.0 with NaOH followed by incubation at 42° C. The reaction progress was monitored by HPLC using C4 analytical column with a gradient flow of 0-60% B in 30 min, showing reaction completion within 4 h, and was purified by HPLC using preparative column C4 with a gradient flow of 0-60% in 60 minutes to give peptide 35 in 40% yield. A similar procedure to the method described for cyclic peptide 24 synthesis, was applied for the preparation of peptide 36. Then, biotinylated cyclic peptide 37 was prepared as per the mentioned procedure for the synthesis of biotinylated cyclic peptide 31. Next, Cys (Acm) deprotection step was performed according to the mentioned procedure in section 12. The reaction progress was monitored by HPLC using a C4 analytical column with a gradient flow of 0-60% B in 30 min. The reaction was completed within 30 min, quenched with DTT. Following centrifugation, the mixture was injected into HPLC using a semi-preparative C4 column with a gradient flow of 0-60% B in 60 min to give peptide 38 in 20% isolated yield.

### Cell Culture Procedure

HeLa (CCL-2™) and 293T (CCL-2™) cells were cultured in DMEM (high glucose) supplemented with 10% FBS, 0.2 mM L-Gln, and antibiotics (penicillin/streptomycin) in a humidified 37° C. incubator at 5% CO₂. U2OS (HTB-96™) cells were cultured in DMEM (low glucose) supplemented with 10% FBS, 0.2 mM L-Gln and antibiotics (penicillin/streptomycin) in a humidified 37° C. incubator at 5% CO₂. To detach cells from culture flasks, the media was aspirated, and the flask was washed with sterile calcium and magnesium-free PBS before cells were treated with 0.25% trypsin 0.02% EDTA solution and returned to the incubation chamber for 4-5 min. Trypsin was quenched by adding the supplemented media. The cell suspension was collected, and pelleted (2 min at 1,000×g). Media then aspired, and the cell pellet was resuspended in fresh media. The cell density was determined using an automated cell counter (Countess II, Invitrogen) and seeded accordingly.

### Cell Uptake Studies

Cells were seeded for 24 h on ibidi 8 well μ-slides treated with poly-L-lysine to reach ˜90% confluency. Cells were then washed three times with warm PBS followed by incubation for 1 h with warm serum-free medium (DMEM) containing peptides. Thereafter, cells were washed two times with warm PBS. Prior to imaging, cells were washed with an optical culture medium and stained with Hoechst (2 μg/ml). Live cell CLSM images were captured using Confocal Zeiss LSM 710 equipped with 40×NA 1.2 water immersion objective lens using a 1 AU pinhole settings. Different lasers were used for the different tags (Hoechst, TAMRA, FITC), and during analysis, the μ-slide was kept at 37° C. in a humidified chamber.

### Induction of Histone H2AX Phosphorylation

Accumulation of histone H2AX phosphorylated at serine-139 (γ-H2AX) was studied by western blot using standard lysis protocol. Briefly, treated U2OS or HeLa cells (3×10⁶) with samples (2 μM cyclic peptide or DMSO) were harvested and lysed in 100 μl of hot lysis buffer (50 mM Tris-HCl buffer, pH 8.0, 150 mM NaCl, 0.5% Nonidet P-40, and 1 mM phenylmethylsulphonyl fluoride) for 1 h on ice. Samples were then incubated for 15 min at 95° C. and after the solution get into RT, samples were again incubated with benzonase nuclease at 37° C. for 20 min. Solutions were centrifuged at 15,000 rpm for 20 min at 4° C. Cell lysates containing 20 μg of whole-cell protein were separated on 12% SDS-PAGE gels and blotted onto polyvinylidene fluoride (PVDF) transfer membranes. After blocking with a non-protein InstaBlock buffer, the membrane was incubated overnight at 4° C. with anti-γ-H2A.X (phospho S139), a primary antibody against phospho-H2AX (1:1500). After washing with tris-buffered saline tween 20 (TBST) 3 times, secondary anti-rabbit IgG (HRP) antibody for 2 h at RT. Finally, crescendo western HRP solution was added to the membrane, and blots were imaged in the Fusion-400 ECL detection system. The γ-H2AX signal at 15 kDa in the blot was quantified using Fiji software. Anti-H2A.X was used as a loading control.

### DNA Damage Study by Comet Assay

### Sample and Slide Preparation

The Comet Assay was performed following the protocol provided by Abcam Comet Assay Kit (ab238544), with few modifications. In brief, U2OS cells were cultured and treated with peptides similar to previous conditions described for the examination of histone H2AX phosphorylation. After sample treatment for 8 h in serum-free medium, the cells were removed from 60 mm dish by scraping. Thereafter to get the cell pellet, the cell suspension was transferred to a conical tube and centrifuge (2,600 rpm for 3 mins) and was washed twice with ice-cold in ice-cold phosphate-buffered saline (PBS, without Mg2+ and Ca2+). After cell counting, cells were resuspended in ice-cold Mg2+ and Ca2+ free PBS to have 1×10⁵ cells/ml. For slide preparation, the low-melt comet agarose was pipetted onto the supplied 3-well comet slides to obtain a base layer and was incubated for 20 min at 4° C. Then, the cell samples were mixed well with the comet agarose at 1:10 (v/v) at 37° C. and the suspension immediately transfer 75 μL gently onto the top of the base layer without disturbing the base layer. The slides were incubated again for 20 min at 4° C. To avoid ultraviolet light damage to cell samples, all the procedures were performed under minimal light conditions.

### Alkaline Electrophoresis

The immobilized cells in the slide wells were lysed by alkaline lysis solution (Triton X-100 (1:100), DMSO (1:10), 2.5 M NaCl, 100 mM Na3EDTA, 10 mM Tris Base, pH 10). The slides with embedded cells were transferred to a container containing pre-chilled alkaline lysis solution and incubated overnight at 4° C. in dark. Then after, the solution was replaced with the pre-chilled alkaline electrophoresis buffer (300 mM NaOH, 1 mM Na3EDTA, pH>13) and leftover for the next 30 mins at 4° C. in the dark. The slides were directly transferred horizontally to the electrophoresis chamber and filled the chamber with pre-chilled alkaline electrophoresis solution. Then, electrophoresis was done at 1 V/cm for 30 mins with a constant current setting of 300 mA. After completion of electrophoresis, the slides were horizontally transferred to a container containing pre-chilled DI H₂O for 2 mins. The slides were washed similarly twice more. Finally, the slides were horizontally transferred to a container containing cold 70% ethanol and incubated for 5 min. Consequently, slides were horizontally removed from the 70% Ethanol and allowed to air dry for the next 2 h at RT. The DNA was stained with provided vista green DNA dye for 15 min in the dark at RT. The slides were then prepared for microscopy analysis.

### Comet Analysis and Quantification

The images of the cells with comets were taken by a fluorescence microscope (Axio Observer Z1 LSM 700, Zeiss) with a 63× Plan-APOCHROMAT 63×/1.4 oil DTC objective (Zeiss) and a camera (AxioCam MRm, Zeiss). The ‘Tail Moment’ has been suggested to be an appropriate index of induced DNA damage in considering the migration of the genetic material. The tail moment intensity profile was analyzed using the “OpenComet” software plugged-in to Fiji. The fluorescence signals of at least 100 cells per data point were considered for the estimation.

### Transfection of Flag-Tagged-Wt or Mutated RNF168 Genes to Cells and Immunoprecipitation (IP) Against Flag Antibody

pcDNA3-Flag-RNF168 and pcDNA3-Flag-RNF168 deleted MIU1/MIU2 (purchased from Addgene), were overexpressed in Human Embryonic Kidney 293T cells. The transaction proceeded using polyethylenimine (PEI) reagent for twelve hours. Following transfection, cells were treated with 1 μM of cyclic peptide or DMSO for 36 h then exposed to ionizing radiation (IR) of 10 Gy using an X-ray machine (CellRad). After recovery for 6 h, cells were harvested and lysed using IP buffer (50 mM HEPES, pH 7.4, 100 mM NaCl, 0.5% NP-40, 10 mM EDTA, 20 mM beta-glycerophosphate) containing buffer supplemented with protease inhibitors. Finally, proteins were immunoprecipitated using flag antibody and subjected to western blot analysis. In this, protein A/G PLUS-Agarose beads (purchased from Santa Cruz) were washed and blocked at 4° C. for 2 h in IP buffer containing 5% BSA, and whole-cell extracts were prepared using NP-40 lysis buffer and precleared.

### Apoptosis Study Using Annexin V-FITC/PI Double Staining Method

### Sample Preparation

Apoptotic cell death was estimated by using the standard MEBCYTO® Apoptosis Kit (MBL) protocol.3 In brief, HeLa cells, seeded on were treated with samples (1 μM of peptide 2 or DMSO) for 96 h at 37° C. with 5% CO₂. After sample treatment, the cells were harvested from the 60 mm dish by trypsinization and centrifuge at 2,600 rpm for 4 mins. The cells were washed once with phosphate-buffered saline (PBS, without Mg2+ and Ca2+) and were resuspended in supplied binding buffer subsequently stained with Annexin V-FITC and propidium iodide (PI). The annexin V-FITC positive cells were considered as apoptotic cells moreover, the early and late apoptotic cells were distinguished by negative and positive PI signals, respectively. The inventors could not proceed with a higher concentration of the cyclic peptides due to the solubility issues in the buffer medium.

### Flow Cytometry Measurement

The different populations were analyzed using a CYTEK Aurora flow cytometer. The final fluorescence of Annexin V-FITC was obtained in 20,000 cells for the sample analysis in each independent repetition. The unstrained and single strained cells were analyzed as a reference control. All the treated samples were unmixed after measurement. Finally, the unmixed data files were analyzed using FCS Express 6 software.

### Cell Cycle Analysis

Cell cycle analysis was performed using FxCycle™ PI/RNase Staining Solution. HeLa cells were treated similarly as previously (section 23). Following detachment, the cells were washed twice with cold PBS and then fixed with 70% ice-cold ethanol at −20° C. overnight. The fixed cell pellets were washed twice with cold PBS and then incubated with PI/RNase staining solution on ice for 30 min in the dark. Finally, flow cytometry (CYTEK Aurora) analysis of cell cycle was carried out to check the event of cell arrest considering 15,000 cell count for all the samples. The cells at various cell cycle phases were quantified using standard heat plot analysis using FCS Express 6 software.

### Pull Down and Proteomics Analysis

The cell lysate suspension preincubated with biotinylated peptide 31 was subjected to pull down from streptavidin beads by utilizing standard protocol with few modifications. In brief, U2OS cells (3× 10⁶) were collected as cell pellets after trypsinization. Instantly, the lysis buffer (0.5% NP-40, 150 mM NaCl, 50 mM HEPES pH 7.5, 1 μM NMM, and 1 μM IAA) was added to the pellet for 30 min on ice then centrifuges for 15 min at 4° C. The cell lysate suspension was divided equally into two parts and incubated overnight at 4° C. on a rotating wheel with the biotinylated peptide 31 or 38. The streptavidin agarose beads (High-Capacity Streptavidin Agarose Resin, Thermo Scientific) were equilibrated with lysis buffer 3 times under shaking conditions. The washed beads were added to each treated suspension and then incubated for 1 h at 4° C. The beads were washed five times with a wash buffer containing PBS pH 7.5 and the protein complexes were eluted by heating for 5 min at 95° C. with reducing buffer containing DTT. The eluted mixtures were examined by western blot using Anti-Ub (Lys63-specific) or Anti-Ub (Lys48-specific). For positive control, 0.5% v/v of the input was included.

For the Proteomics analysis, the eluted solutions for both the samples (peptide 31 and peptide 38;) with identical lysate concentrations were used. The protein components were cleaved with trypsin and analyzed by the LC/MS using the QE plus (Thermo) mass spectrometer. The data was analyzed using the Proteome Discoverer 2.4 vs the human uniport database, with 1% FDR and at least 2 identified peptides. The ratio between the peptide samples elutes protein abundance was calculated and specific proteins (at least 2-fold more than the control) were taken into consideration.

TABLE 2
Various enriched terms by 31, classified based on their
specific role in cellular processes or functions

| | Enriched terms of proteins |
|---|---|
| Cellular process or functions | (Identified by their gene names) |
| DSB repair | TRIP13, XRCC3, PMS2, MLH1, |
| | SMARCAD1, FANCD2, RAD50, |
| | RAD51, ATM, HUS1, APBB1, |
| | TONSL, MMS19, WDR48 |
| Specific Lys63-linked Ub | UBR5, PCNA, BABAM1, and |
| binding | PSMD14 |
| DSBs responsive and works RNF8, | VCP |
| RNF168 dependent manner | |
| Phosphorylates ‘Ser139’ of histone | PRKDC |
| variant H2AX and regulating DSB | |
| response mechanism | |

### Example 1

### Discovery of Cyclic Peptide 1 for K63 Ub Chains Using RaPID Method

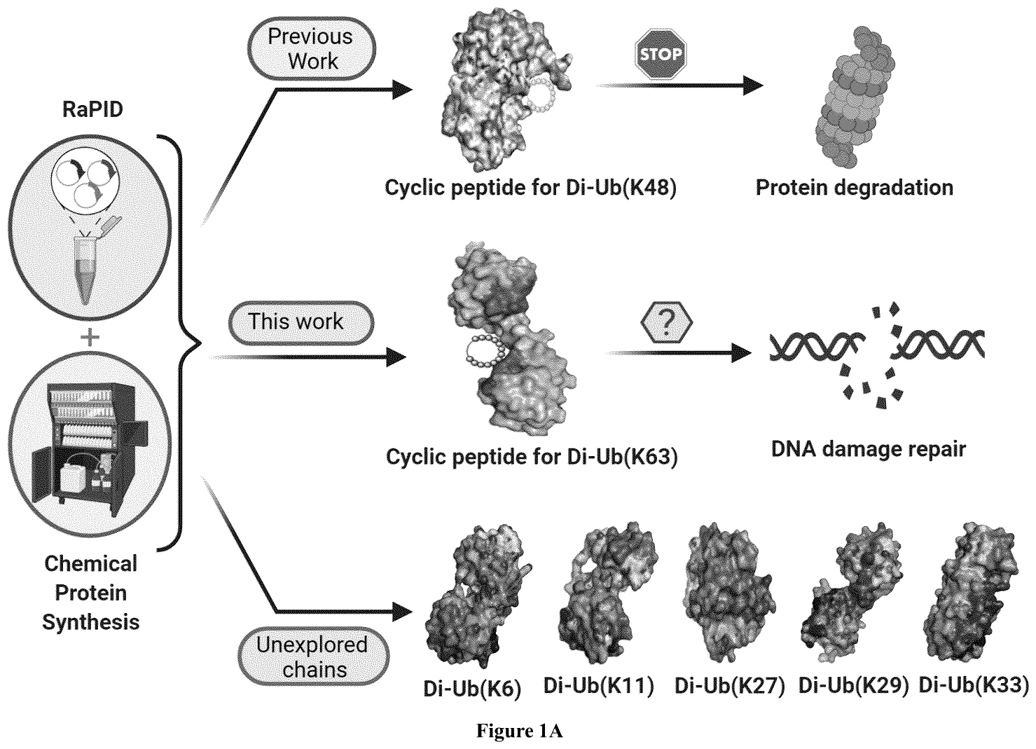

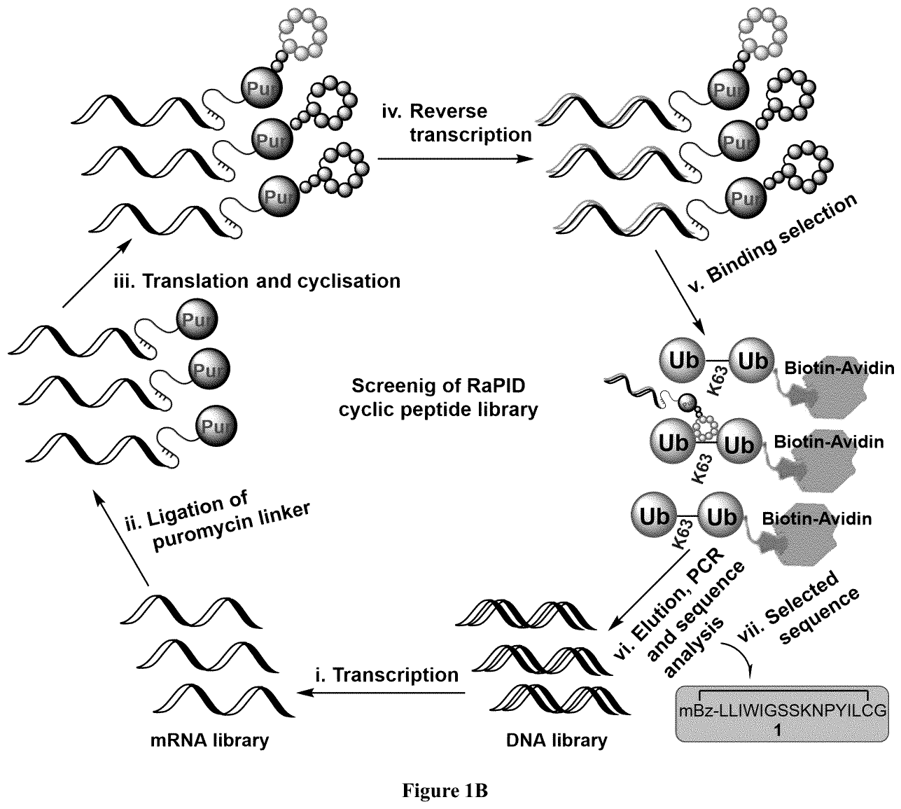

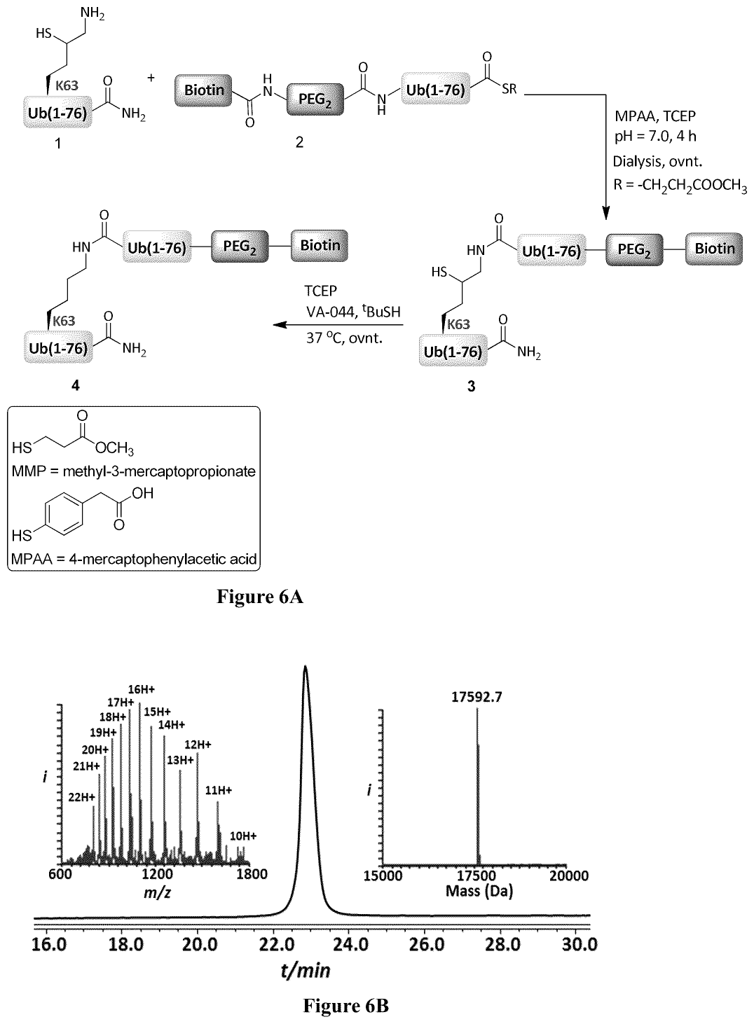

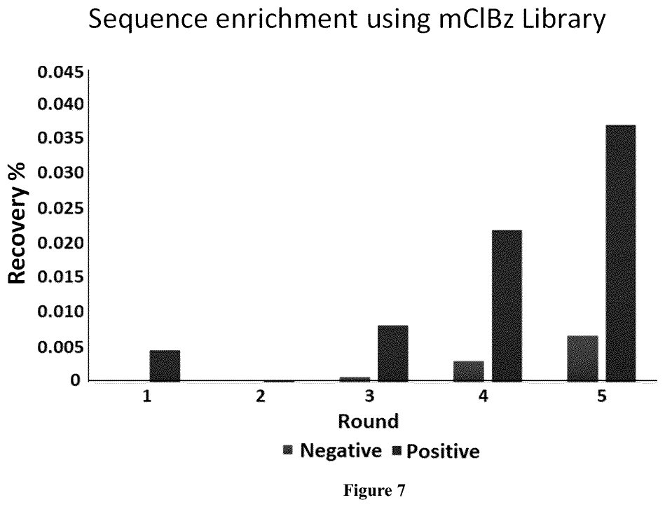

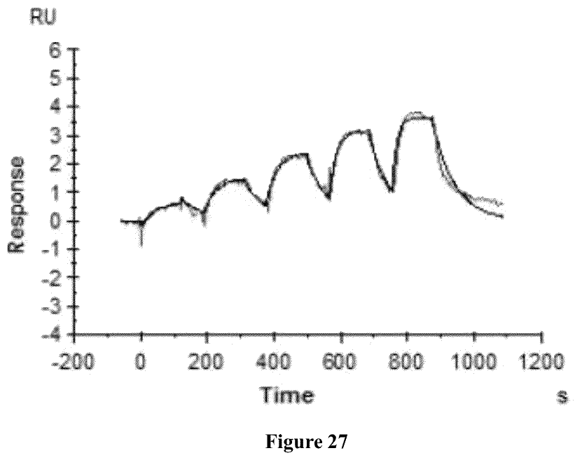

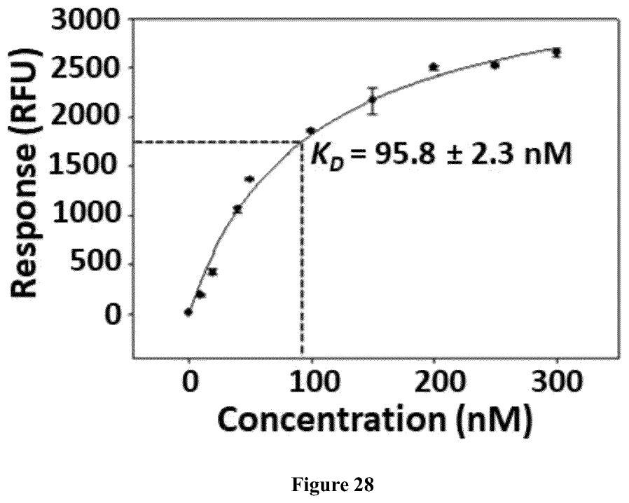

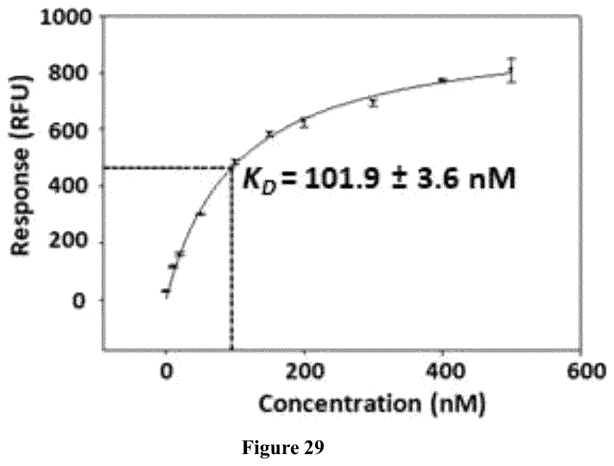

In order to screen for selective Lys63-linked Di-Ub binders, the inventors utilized chemical protein synthesis to prepare Lys63-linked Di-Ub with a biotin tag at the N-terminus of the distal Ub (FIGS. 6A-6B). The inventors then used this chain as bait for the RaPID display to screen a library of trillion mRNA·cDNA-tagged cyclic peptides (FIGS. 1A-1B) against Lys63-linked Di-Ub, providing the first round of selection for cyclic peptides. Mono-ubiquitin (Ub1) binders were then removed by introducing an additional round of selection. After repetition of 2-5 rounds, followed by deep sequencing of the cDNA library, this led to the discovery of a highly specific and tight binding de novo cyclic peptide 1 (CP1) (FIG. 7) with a low-nanomolar dissociation constant (KD) value of 16 nM, as measured by surface plasmon resonance (SPR) (FIG. 27).

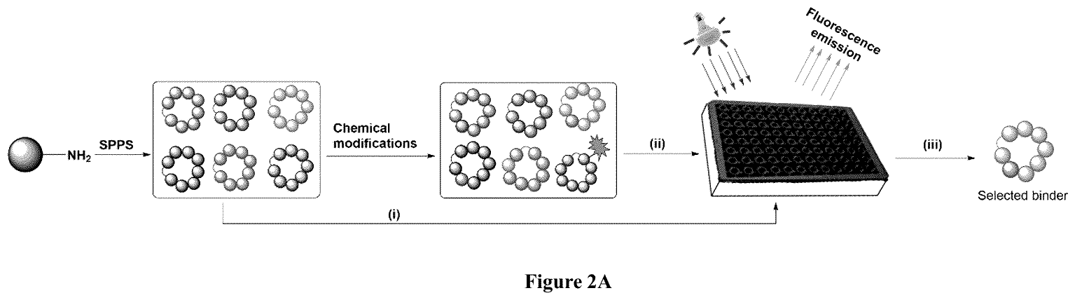

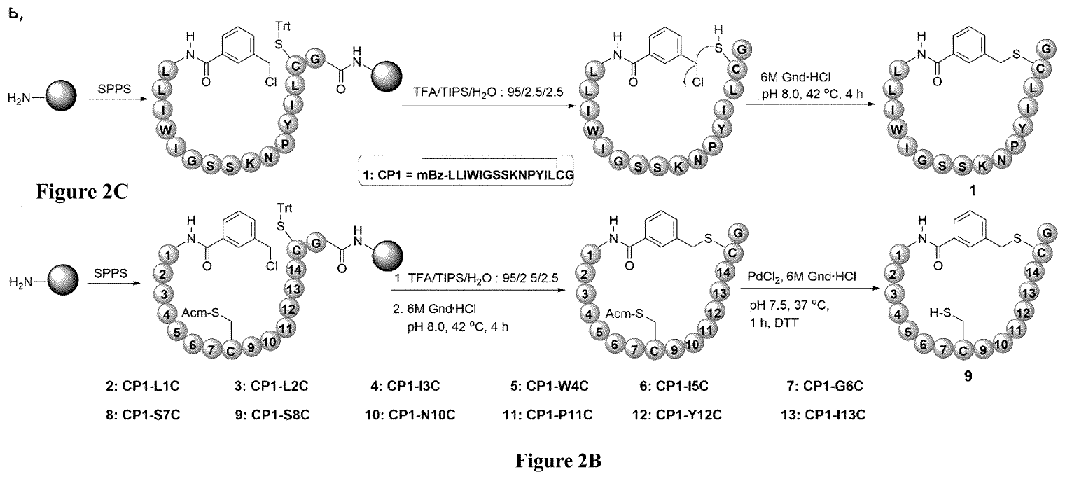

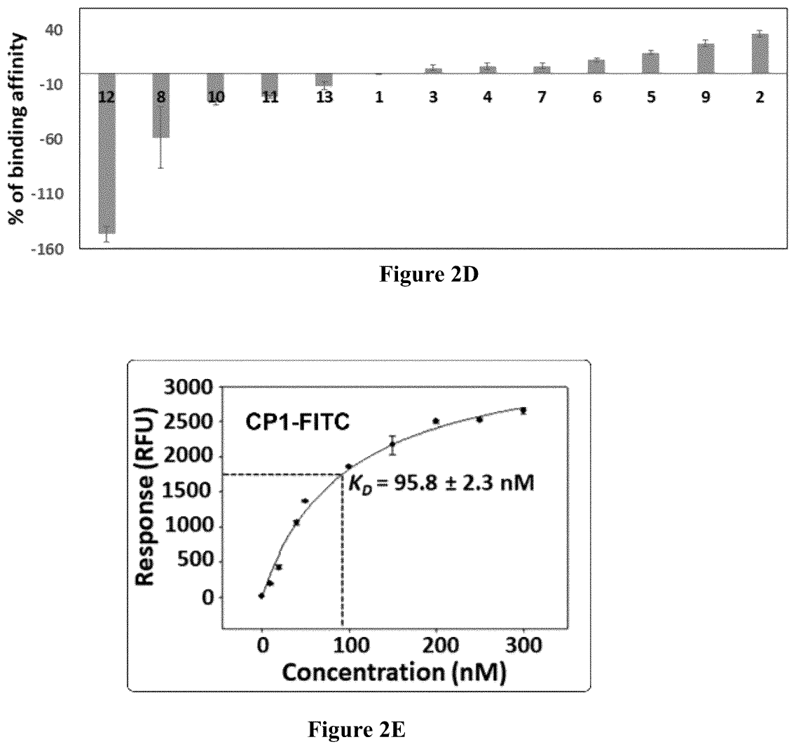

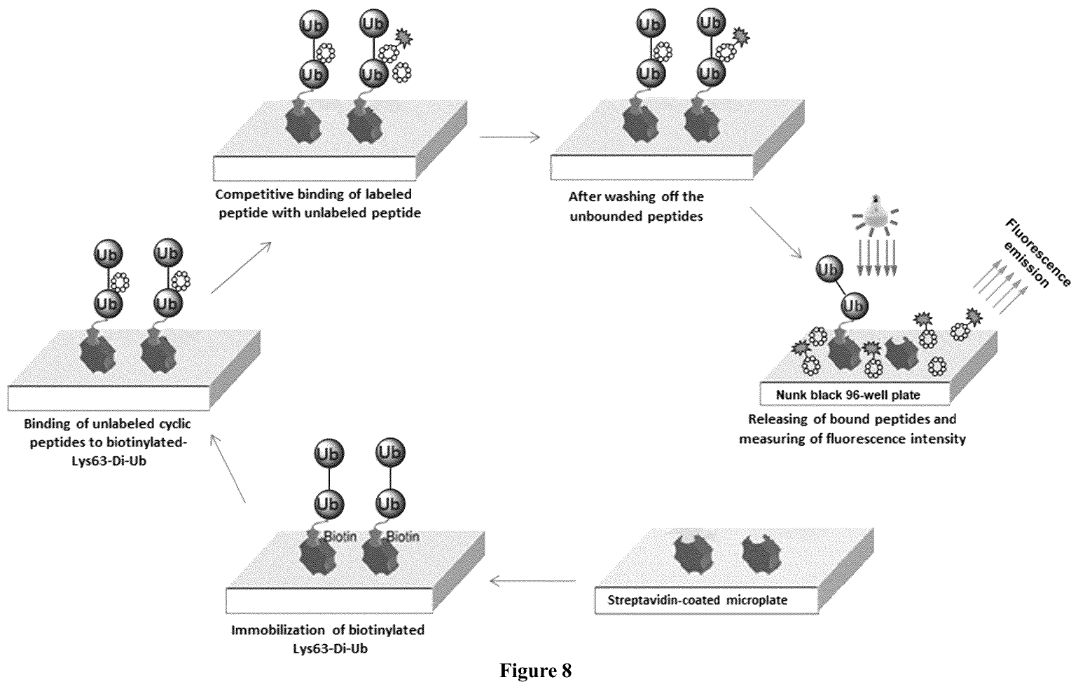

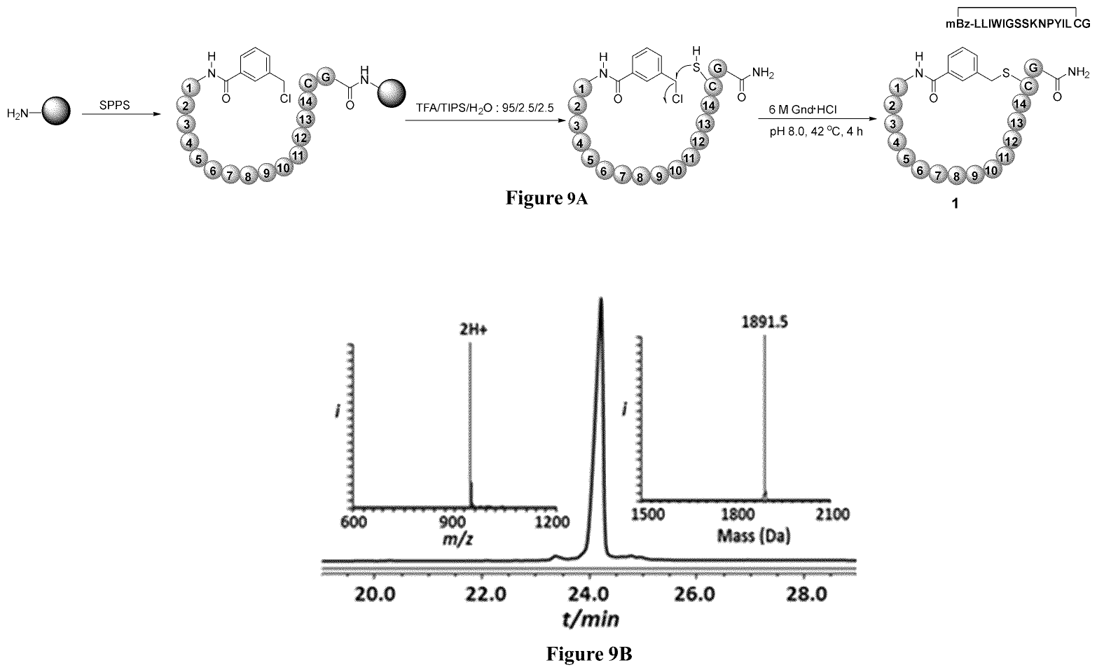

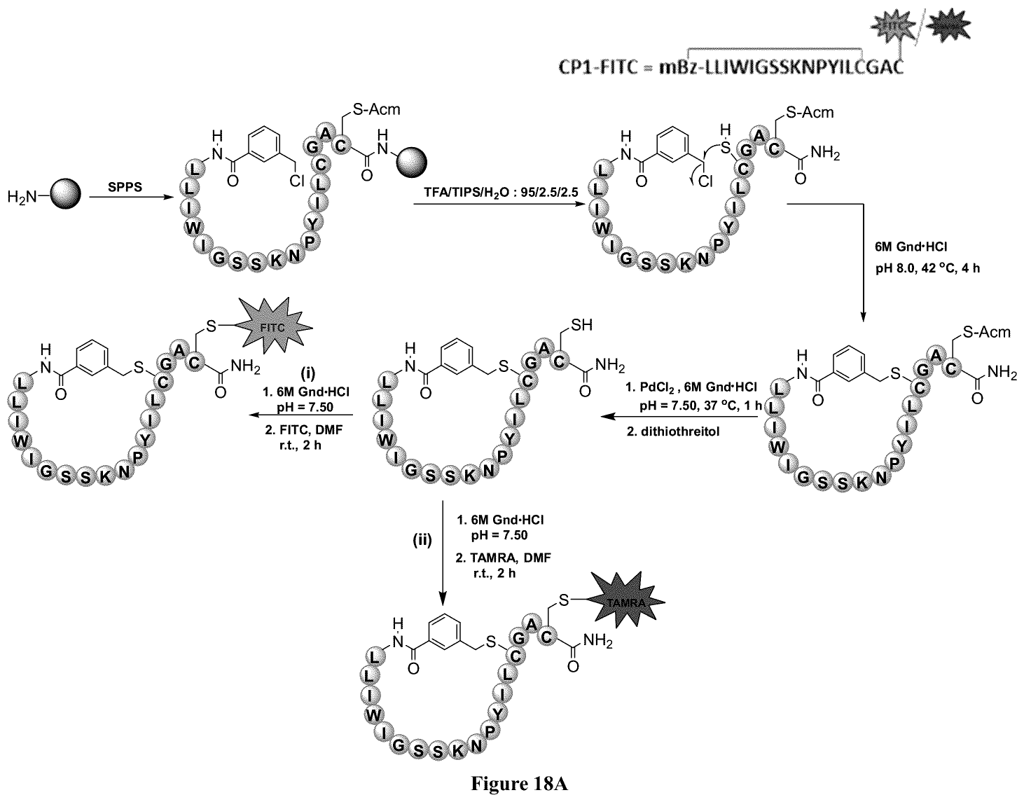

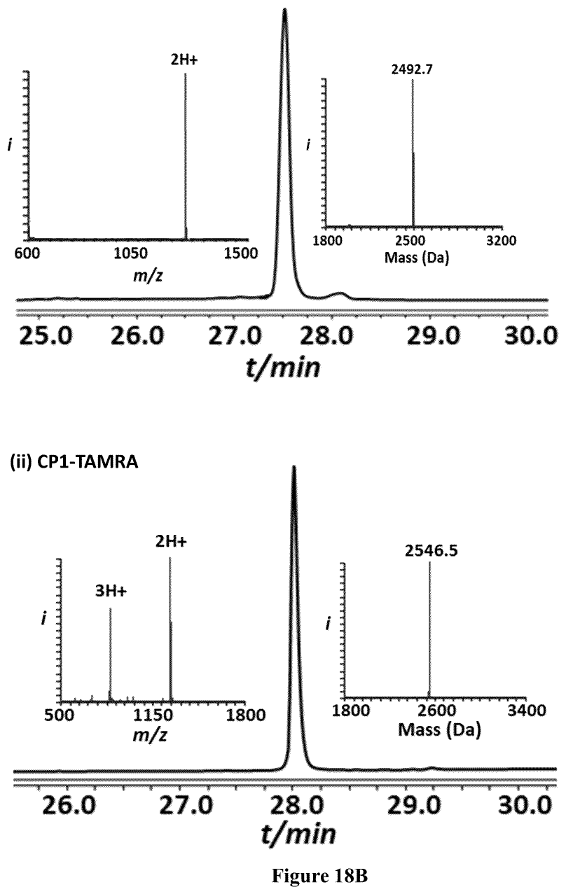

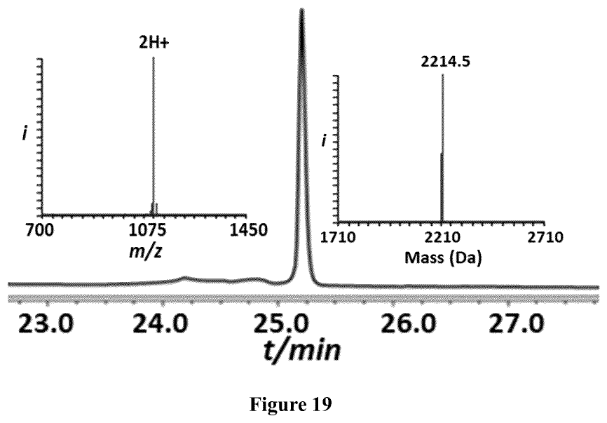

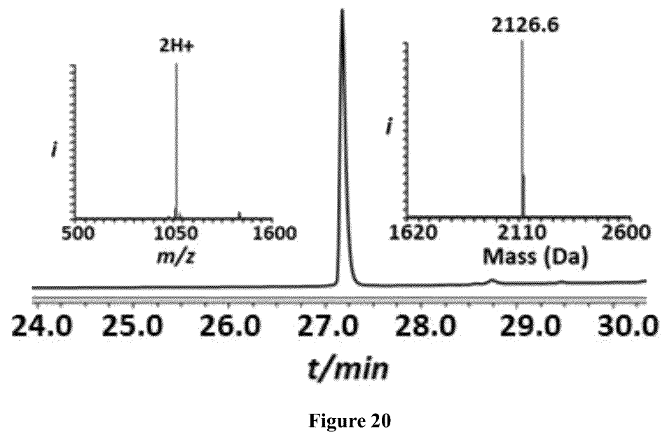

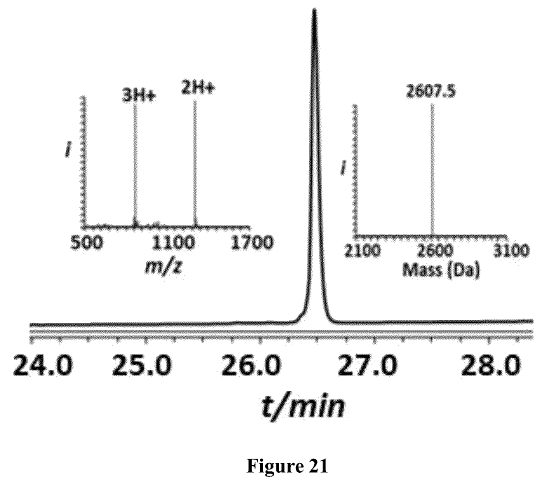

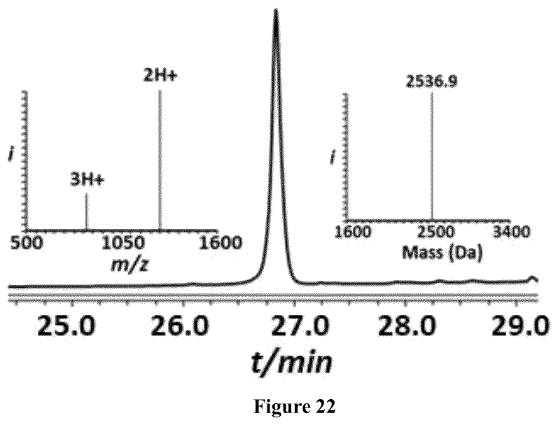

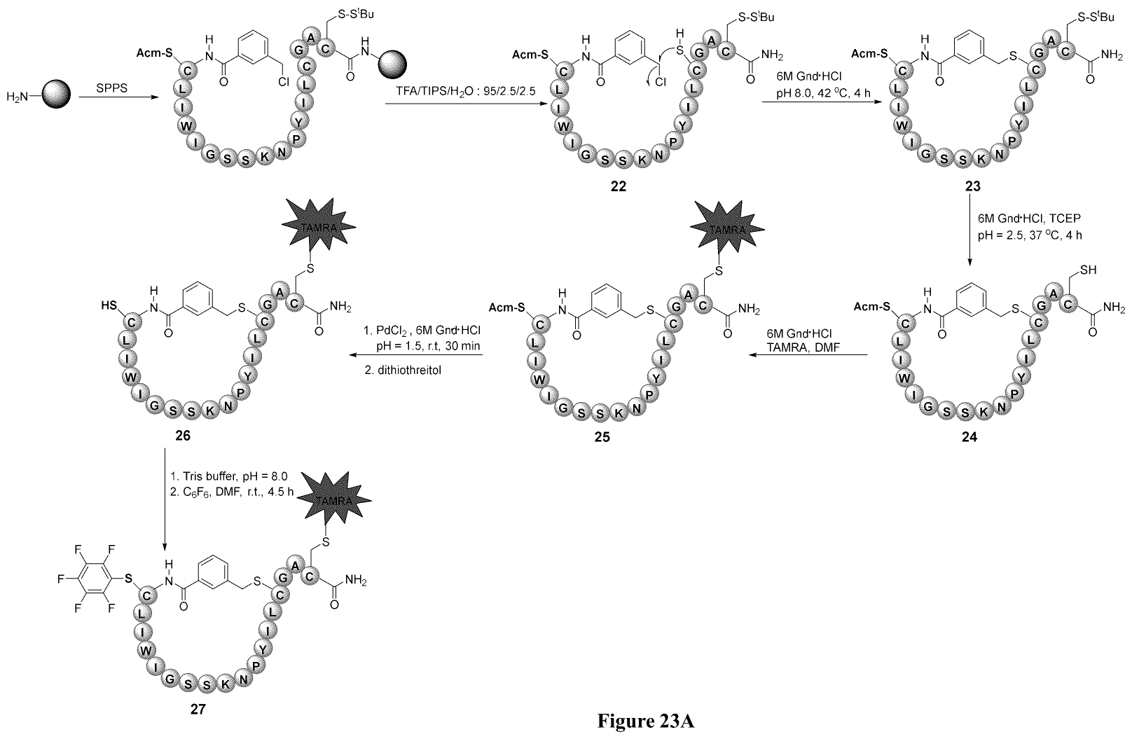

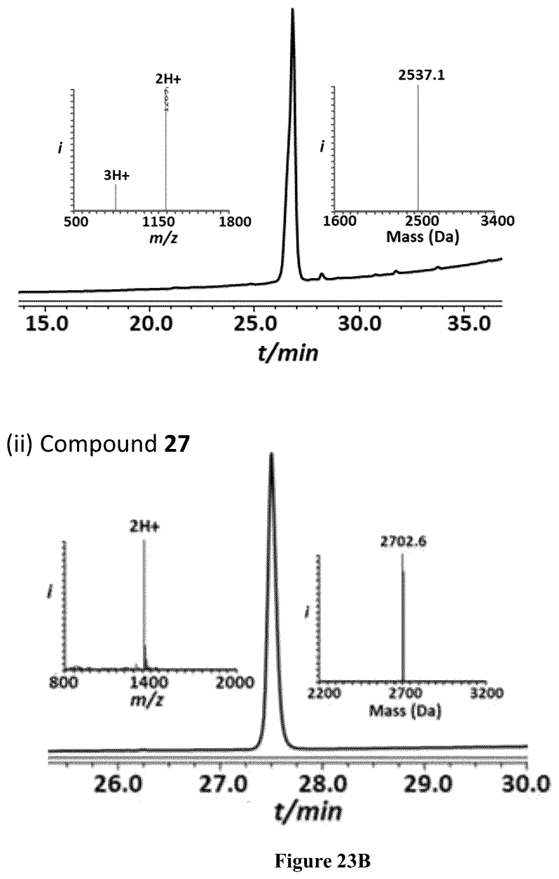

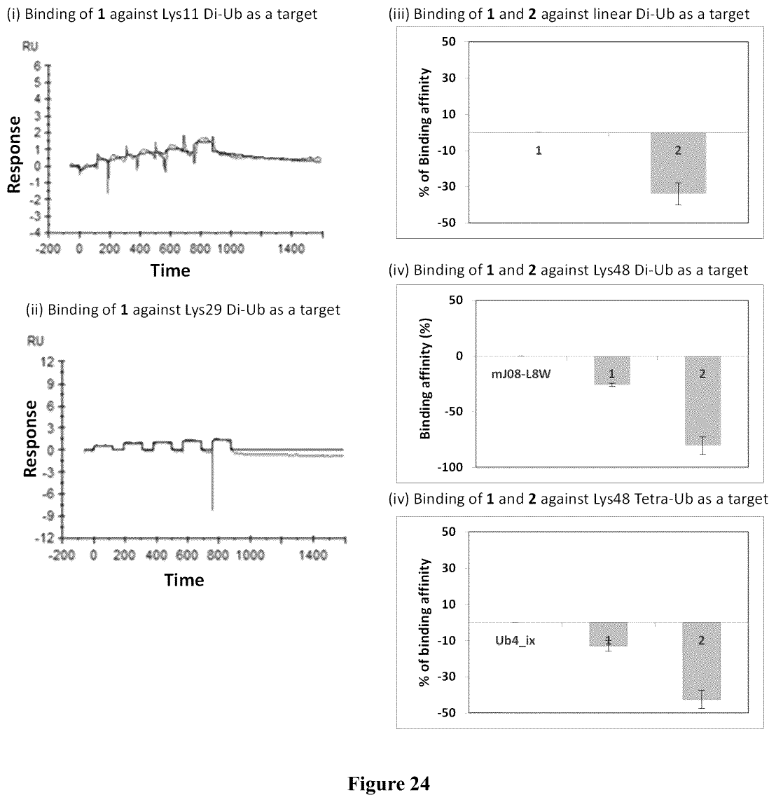

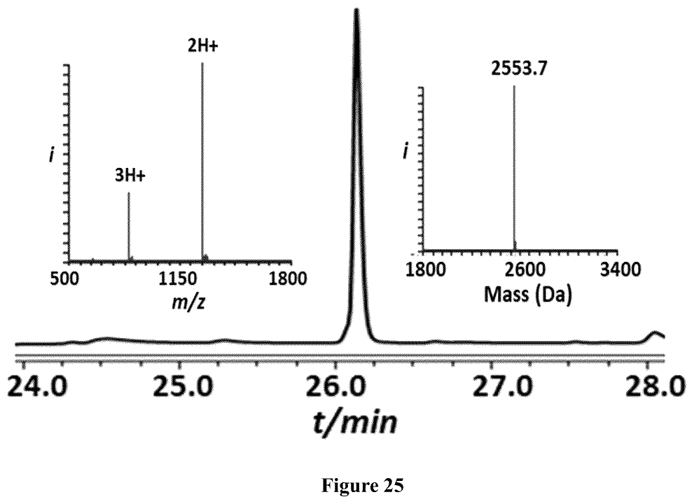

For further screening and determining the relative binding affinity of additional chemically modified cyclic peptides based on the lead compound, the inventors employed their recently developed fluorescence-based assay (FIGS. 2A and 8). Therefore, fluoresceine-tagged cyclic peptide 1 (CP1-FITC; FIG. 18) was prepared, and a KD value of 95.8±2.3 nM was determined (FIG. 2E).

### Example 2

### Preparation and Characterization of Cyclic Peptide 1 Derivations

In the RaPID system, peptide cyclization occurs via the selective nucleophilic attack of the thiol side chain of Cys located at the C-terminal region, on the non-amino acid initiator, such as chloroacetyl (ClAc), 3-(chloromethyl)benzoic acid (mClBz), or 4-(chloromethyl)benzoic acid (pClBz). Notably, adding any additional Cys in the sequence when using this approach for subsequent functionalization is not possible due to the lack of chemoselective cyclization.

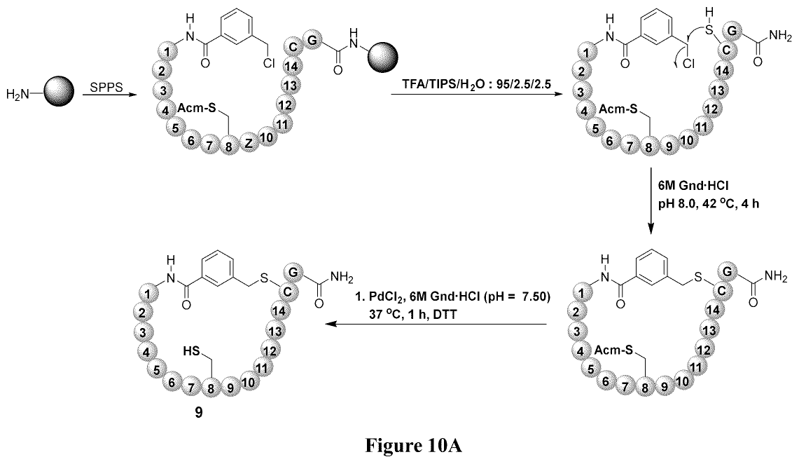

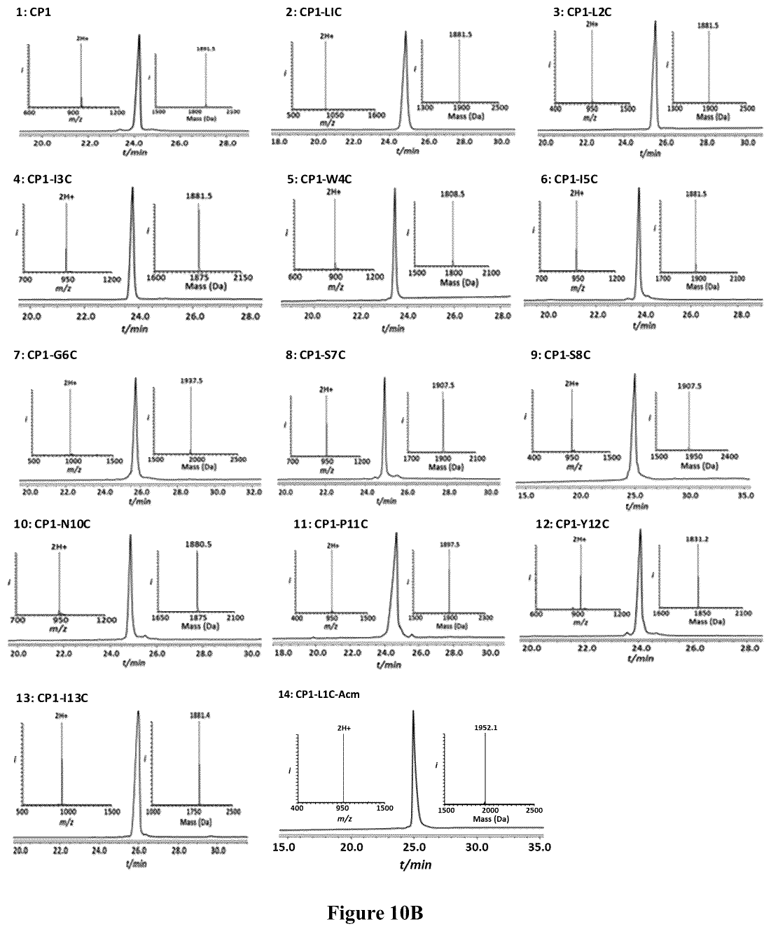

To expand the inventors' library by chemical mutagenesis using chemical Cys modification, without interfering with the cyclization step, they employed Fmoc-solid-phase peptide synthesis (Fmoc-SPPS) to prepare derivatives of cyclic peptide 1 bearing orthogonally protected Cys with acetamidomethyl (Acm) at various positions (FIGS. 2B, 10A-10B, and Table 1). Global deprotection and cleavage leave the trityl protecting Cys residue with a free thiol group, which undergoes cyclization with the mCIBz moiety. In the following step, Acm was removed via PdCl2, to give the free thiol, as shown in the representative example for the synthesis of cyclic peptide 9 (CP1-S8C; FIG. 2C). Using the same strategy, we prepared the additional derivatives of cyclic peptide 1, each having a second Cys residue at different positions (FIGS. 10A-10B, and Table 1).

With cyclic peptides 2-13 in hand, the inventors then examined their peptides affinity for Lys63-linked Di-Ub using their fluorescence-based assay. The relative binding of each cyclic peptide was normalized to cyclic peptide 1, as a reference, where the binding affinity of each cyclic peptide is inversely proportional to the FITC fluorescence of CP1-FITC. Using this assay, the inventors identified that cyclic peptide 2 is the more effective binder of Lys63-linked Di-Ub compared to 1 (FIG. 2D).

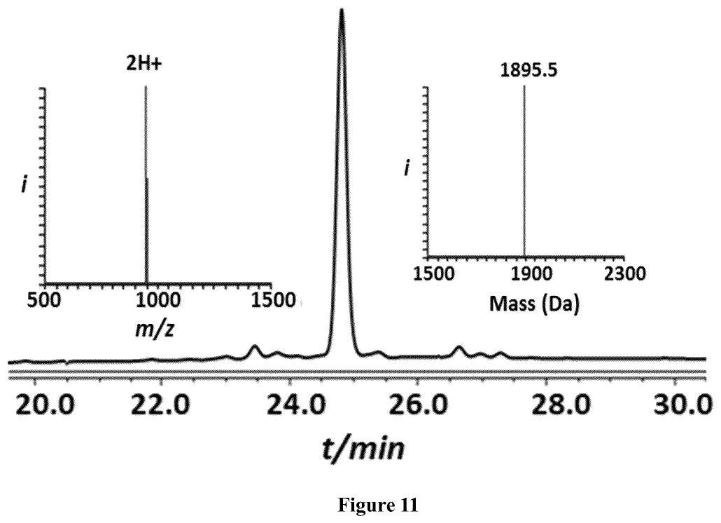

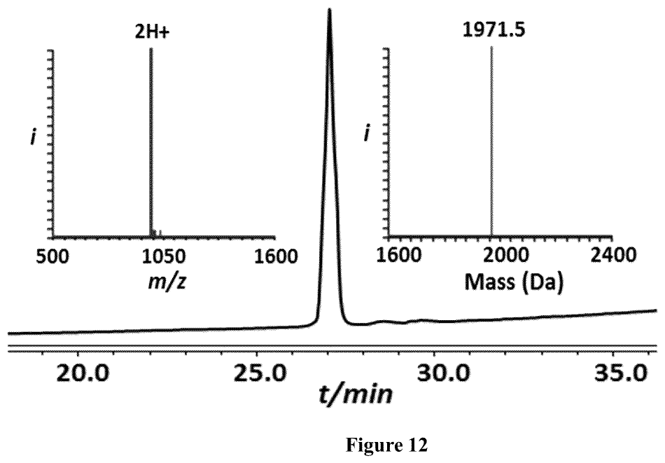

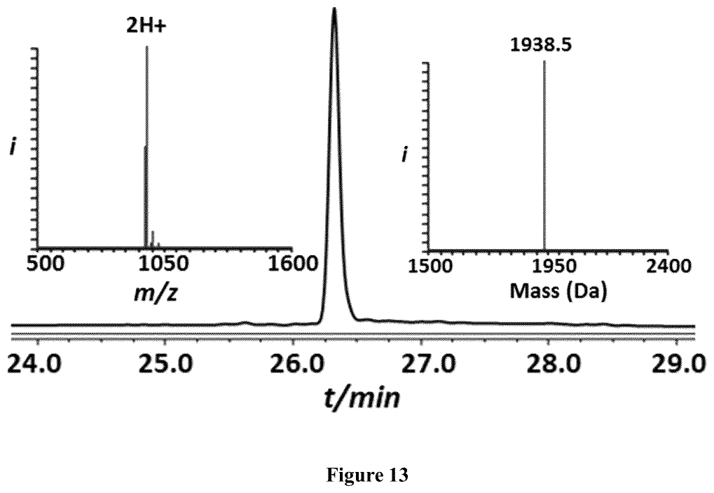

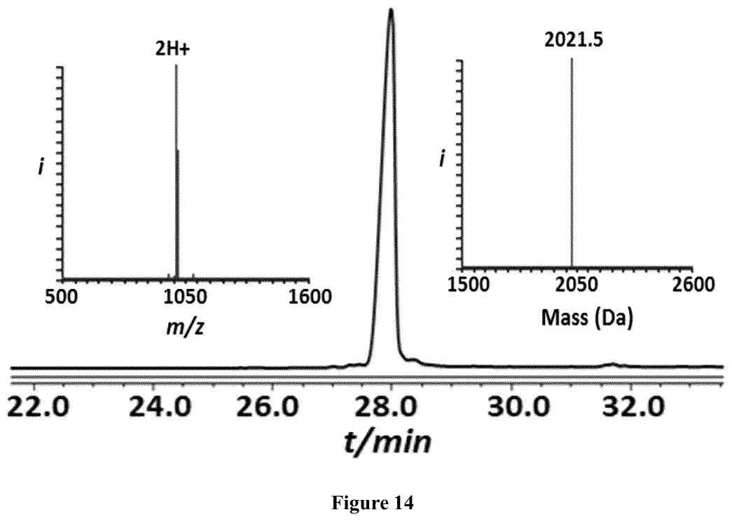

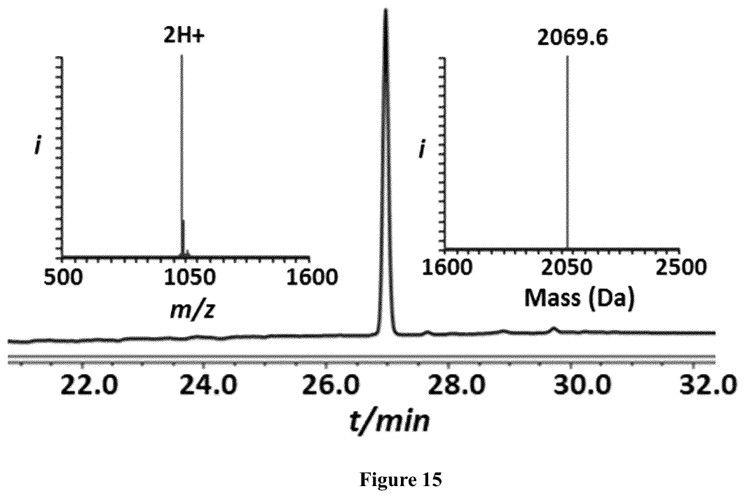

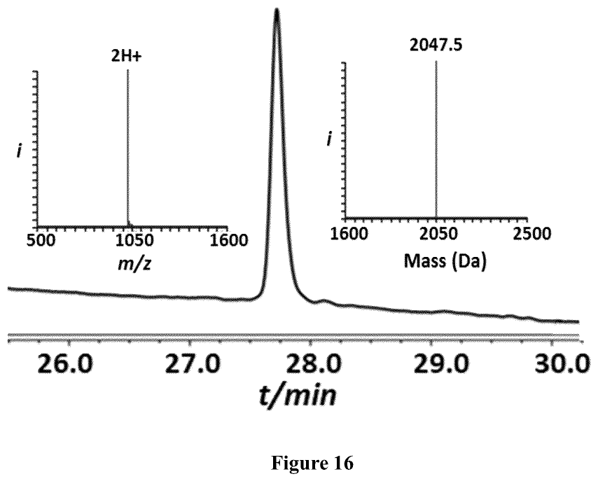

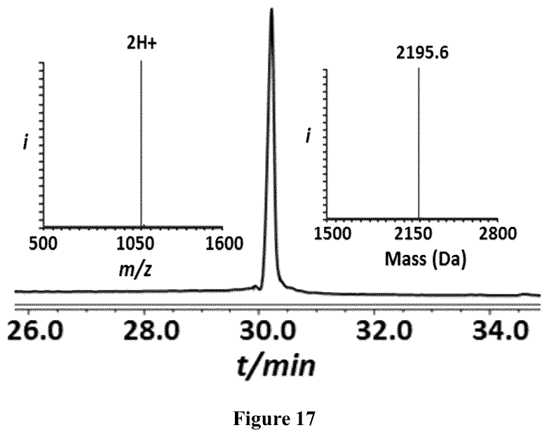

To further attempt the improvement of the binding affinity of cyclic peptide 2, the inventors utilized the advantage of the free thiol in cyclic peptide 2 for chemical modification with different groups. Notably, the alkylation was done in one-pot employing removal of Acm protecting group and treatment with various alkyl halides. This in situ reaction allowed the rapid preparation of five different alkylated (15-19) and arylated (20-21) derivatives of 2 (FIGS. 3A, and 11-17). All the modified peptides showed diminished binding compared to the cyclic peptides 1 and 2 (FIG. 3B).

Next, the inventors investigated the selectivity of cyclic peptide 2 for Lys63-linked Di-Ub over other Ub chain types. Since their lead cyclic peptide 1, had undetectable binding for Lys11 and Lys29-linked Di-Ub chains (FIG. 24), they further investigated the binding affinity of cyclic peptide 2 with the other Ub chains. For this, they compared the binding affinity of 2 and 1 to the reported Lys48-linked Di-Ub and Tetra-Ub binders; mJ08-L8W, and Ub4_ix, respectively, (FIG. 24). The results revealed that 2 has a significantly lower binding affinity for linear Di-Ub, Lys48-linked Di-Ub, and Lys48-linked Tetra-Ub. In all these experiments, cyclic peptide 2 exhibited higher selectivity for the Lys63-linked Di-Ub chain compared to cyclic peptide 1.

### Example 3

### Cellular Effects Examination of Cyclic Peptides Targeting K63 Ub

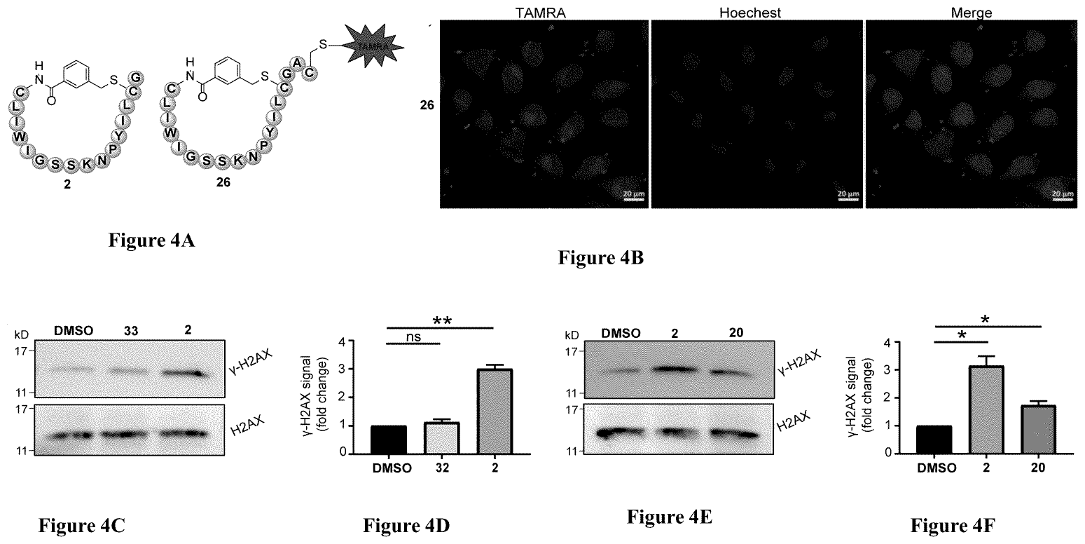

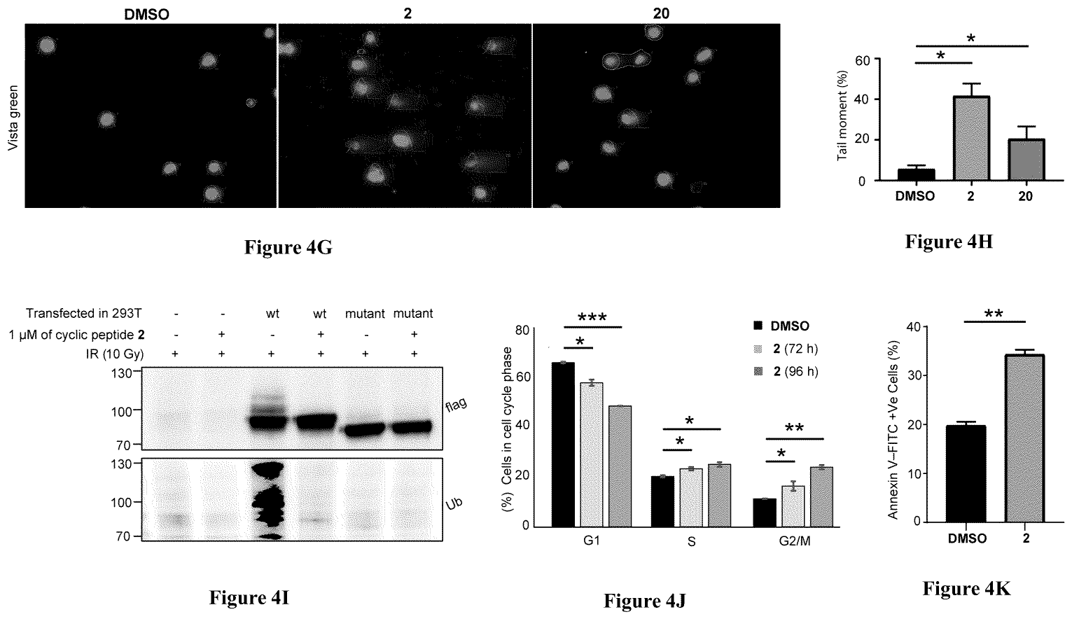

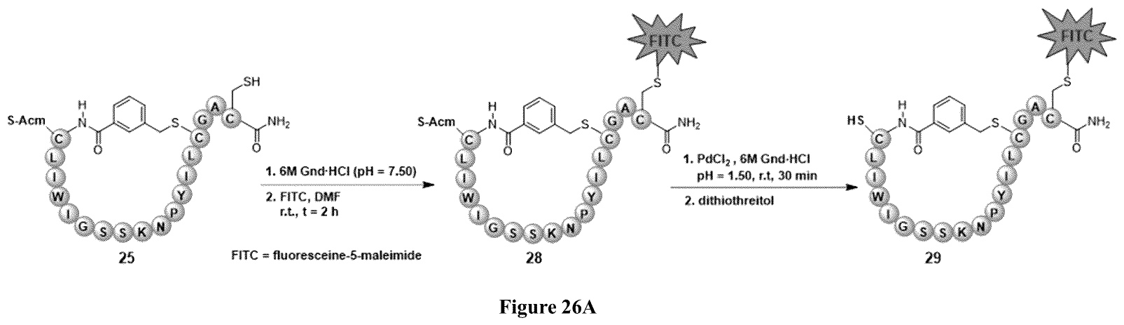

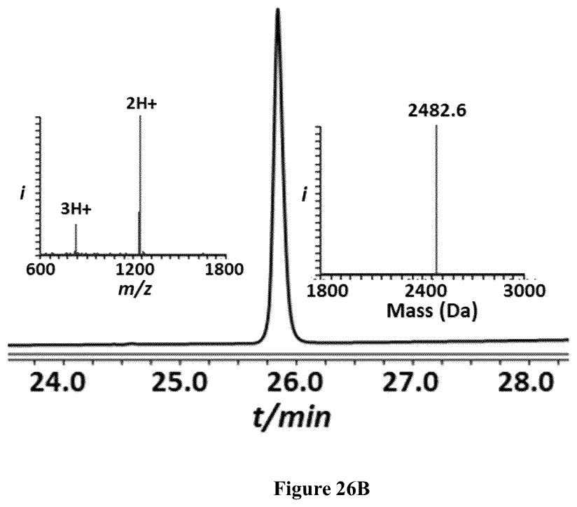

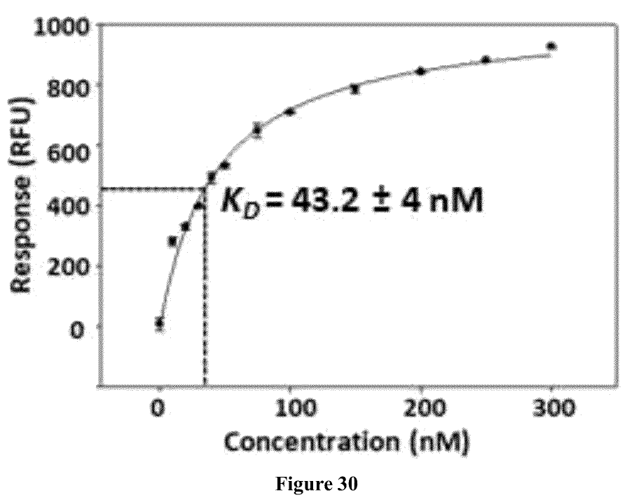

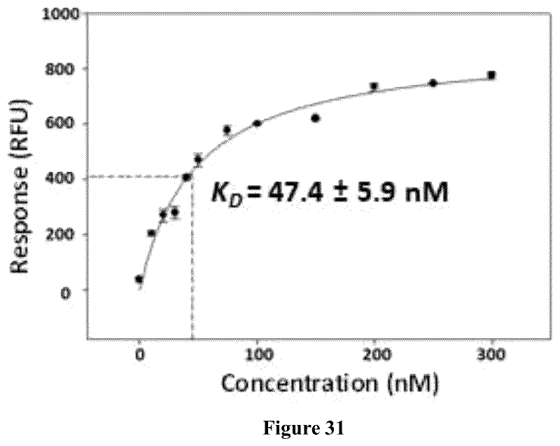

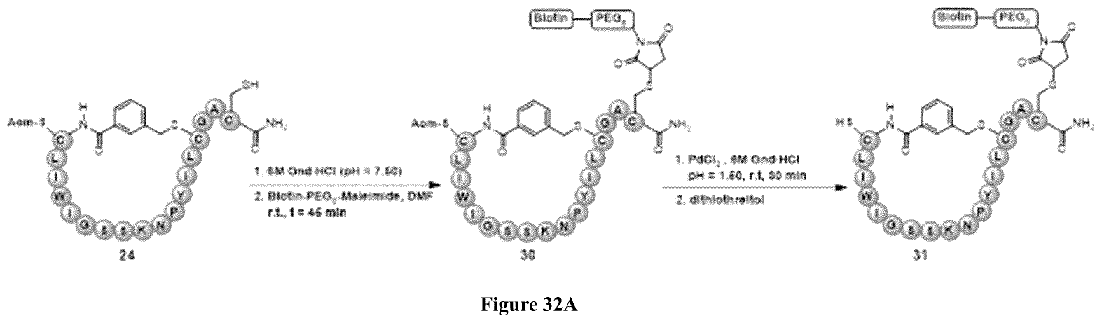

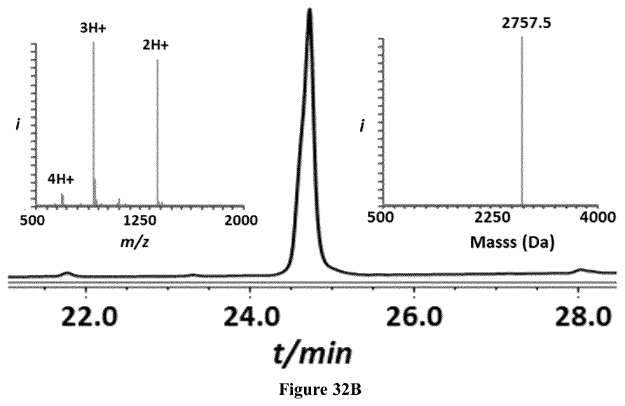

To check the cellular effect of their cyclic peptides, the inventors first aimed to examine the cell-permeability of cyclic peptide 2. For this, they prepared FITC- or TAMRA-labeled peptides 2-FITC (29) (FIG. 26) and 2-TAMRA (26) by incorporating Cys (StBu) to facilitate labeling with the maleimide dyes followed by orthogonal palladium promoted Acm removal to expose Cys1. To examine the fluorophore influence on the binding of cyclic peptide 2, they determined the KD value of cyclic peptides 2-FITC (29) and 2-TAMRA (26) using the fluorescence-based assay as; 43.2±4.0 and 47.4±5.9 nM, respectively, (FIGS. 30 and 31). Their results showed that fluorophore selection (i.e., FITC or TAMRA) does not affect the binding affinity of the cyclic peptides to Lys63-linked Di-Ub. They then measured the delivery of these cyclic peptides to U2OS cells by laser scanning confocal microscopy (LSCM) and confirmed that 2-TAMRA (26) has an efficient cell-permeability (FIGS. 4A-4B, and 35). Notably, they did not observe an efficient delivery for 2-FITC (29) (FIG. 35), suggesting that the choice of fluorescent dye significantly changes the cell-permeability behavior of the labeled cyclic peptides.

After demonstrating the cell-permeability of 2-TAMRA, the inventors aimed to examine if the unlabeled cyclic peptide 2 could modulate cellular pathways where Lys63-linked chains are known to be involved. It has been reported that Lys63-linked Ub chains regulate various cellular pathways, including DNA damage repair (DDR), signal transduction, protein trafficking, and immune response. Mis-regulation of these pathways leads to stress, accumulation of mutations, apoptosis, and cell cycle arrest that can result in various pathological conditions.

Phosphorylation of the Ser-139 residue of the histone variant H2AX, forming γ-H2AX, is the earliest known marker of double-strand breaks (DSBs). Formation of γ-H2AX is followed by the recruitment of DNA repair proteins to DSB sites. Among these proteins, RNF8 and RNF168 ubiquitin E3 ligases generate Lys63-linked Ub conjugates on histone and nonhistone proteins (e.g., 53BP1 and BRCA1) surrounding DSB sites to facilitate repair. The accessibility of Lys63-linked Ub chains in these processes must be influenced by the presence of external modulators. Therefore, the inventors expected that the interaction between their cyclic peptide 2 and Lys63-linked Di-Ub chains could disrupt the interaction between these chains and their endogenous partners and thus inhibit DSB repair, resulting in the accumulation of fragmented DNAs.

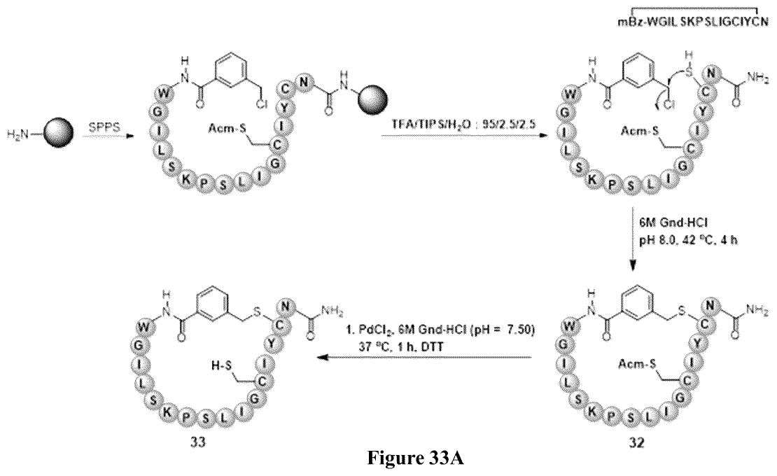

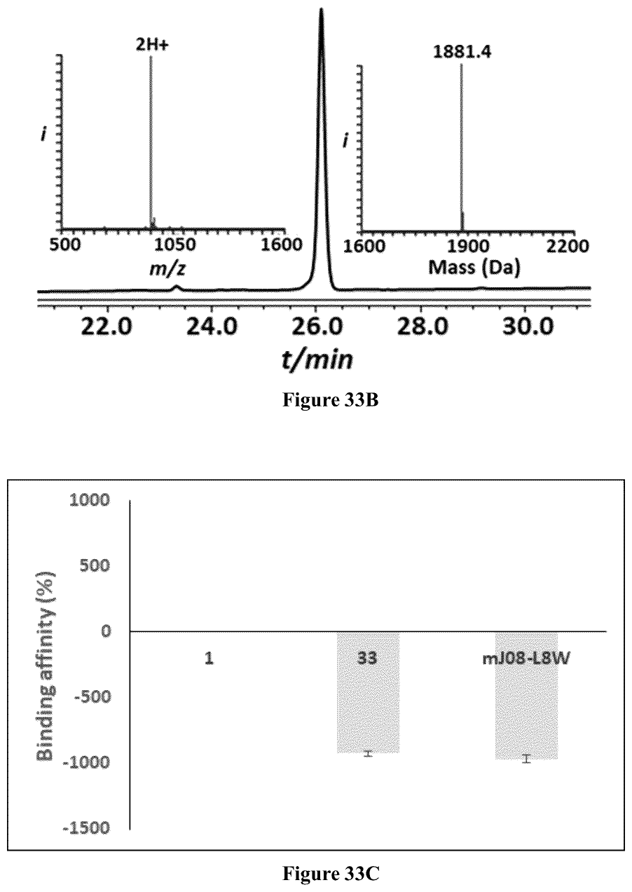

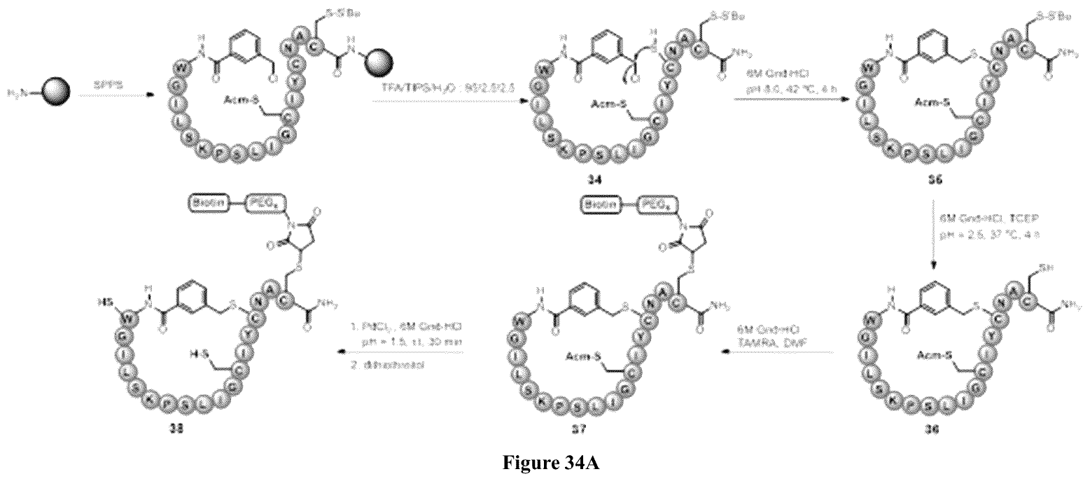

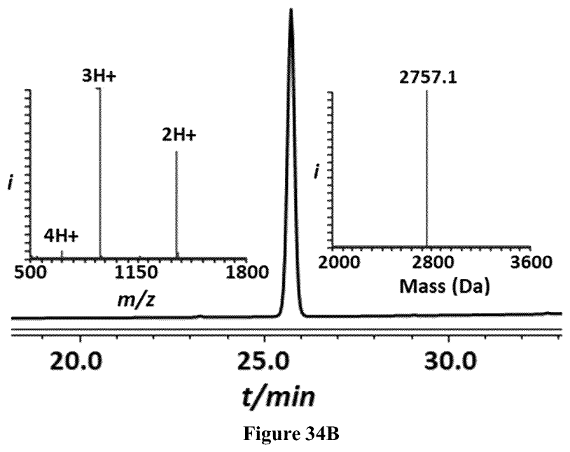

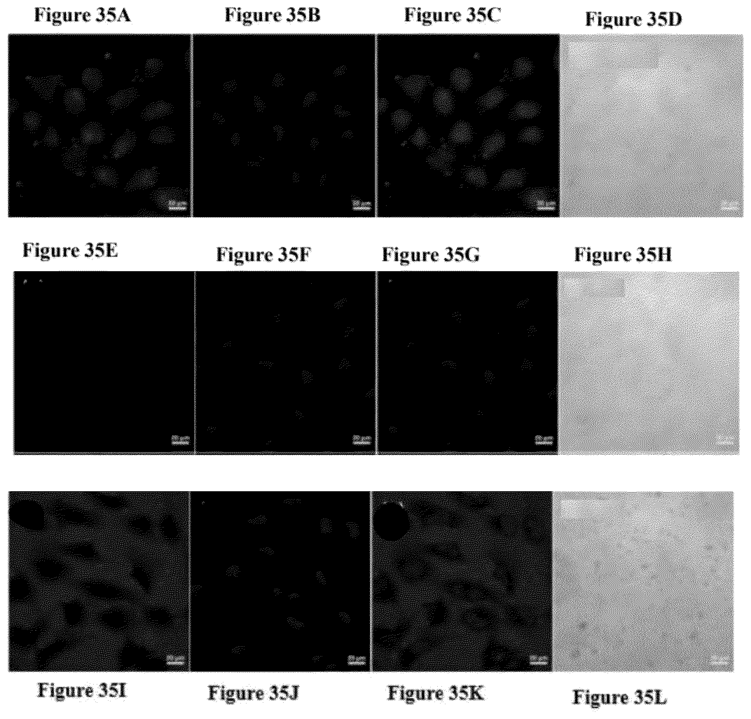

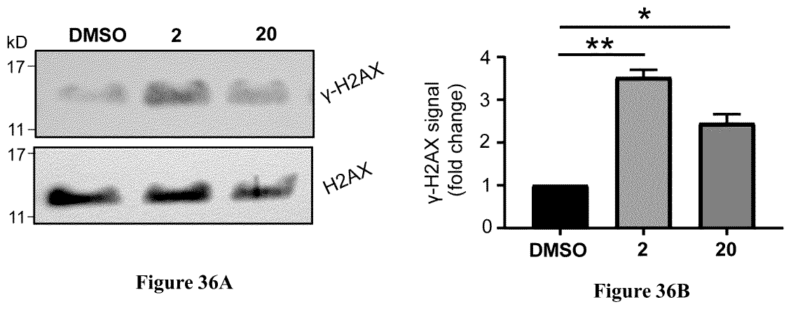

To investigate the effect of the herein disclosed Lys63-linked Di-Ub binder on DSB repair, the investigators treated U2OS and HeLa cells with cyclic peptide 2 and compared the γ-H2AX levels to control cells using western blot. Their results clearly show that the levels of γ-H2AX were increased by 3 and 3.5-fold upon treatment with cyclic peptide 2 in U2OS and HeLa cells, respectively. Importantly, 33 (a scrambled sequence of cyclic peptide 2), which does not have traceable binding affinity to the K63-linked Di-Ub (FIG. 33C), does not alter the levels of γ-H2AX (FIGS. 4C-4D). In addition, when they included cyclic peptide 20 that has a lower binding affinity to the chain compared to 2, which exhibited an increase of γ-H2AX level by 1.8 and 2.4-fold in U2OS and HeLa cells, respectively (FIGS. 4E-4F, and 36). The observed increase in γ-H2AX levels indicates that cyclic peptides 2 and 20 lead to inhibits the repair of DSBs and that this depends on their affinity to Lys63-linked chains.

To further substantiate their findings, the inventors sought to directly measure the integrity of DSB repair at a single cell level using comet assay. The herein disclosed results show that cells treated with cyclic peptide 2 exhibited a significant increase in the amount of fragmented DNA as evident in the “comet-like” vista green signal from the DNA of individual cells (FIG. 4G). Moreover, the relative tail moment, which directly measures the degree of DNA damage, shows a significant enhancement (8-fold) in cells treated with cyclic peptide 2 compared to the control. Similarly, cyclic peptide 20 also induced DNA damage but as in the previous experiment to a lower extent compared to cyclic peptide 2 (FIG. 4H).

At the DSB site, RNF168 accumulates itself as a downstream of RNF8 that interact with ubiquitylated H2A to catalyze the formation of Lys63-linked Ub-conjugates. The MIU motifs (MIU1 and MIU2) in RNF168 mediate its binding to Lys63-linked Ub chains on histones which is necessary to promote DNA damage response. Therefore, to examine the direct binding activity of their developed cyclic peptide 2 to the Lys63-linked Ub chains in cell, the inventors have transfected flag-tagged RNF168 (wt) and MIU1/MIU2 deleted RNF168 (mutant) genes in 293T cells. The transfected cells were treated with cyclic peptide 2 (1 μM) or DMSO for 36 h and exposed to ionizing radiation (IR) (10 Gy) to stimulate the accumulation of DSB with the associated Lys63-linked Ub chains. Immunoprecipitation and western blot analysis using anti-flag antibody showed several additional signals for RNF168 (wt) transfected cells, appears to be RNF168 and its ubiquitinated forms, which further verified by their Ub signals. On the other hand, treatment with cyclic peptide 2, abolished these additional signals of RNF168 and conjugated Ub, which strongly supports the modulation activity of 2 for the Lys63-linked Ub chains in the cells. Importantly, peptide 2's effect was absent in cells that were transfected with mutant RNF168 (FIG. 4I).

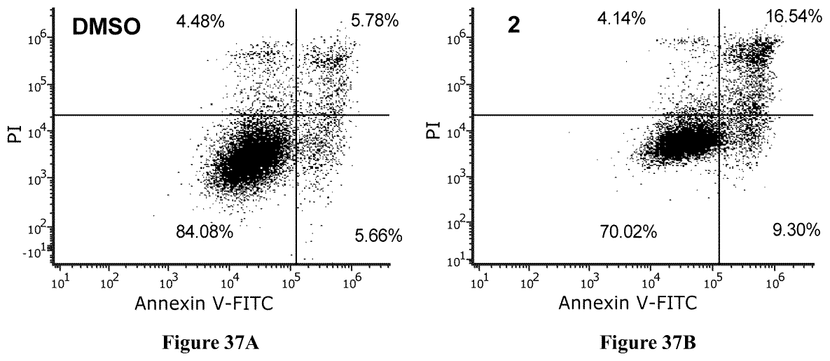

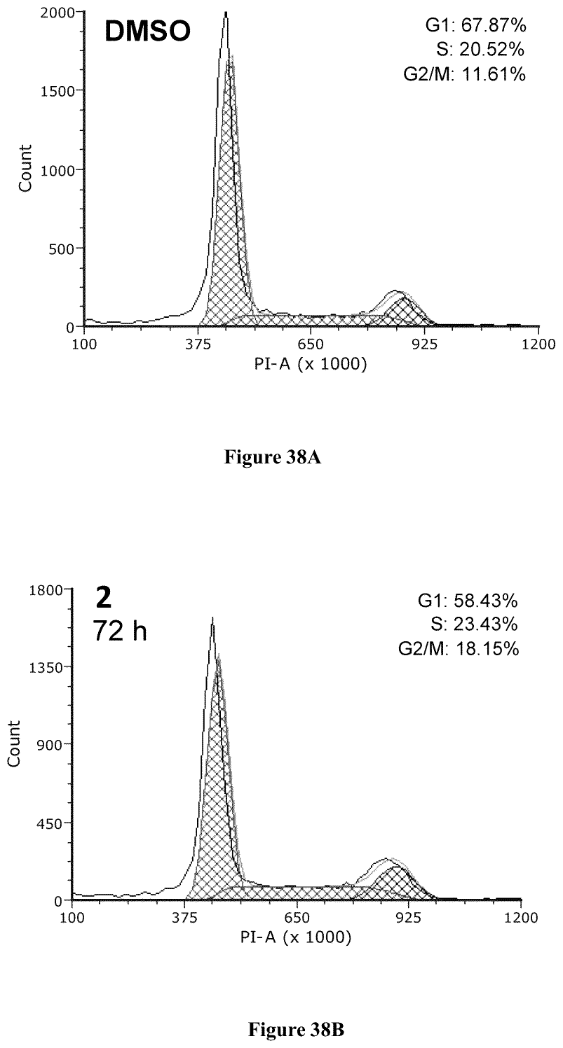

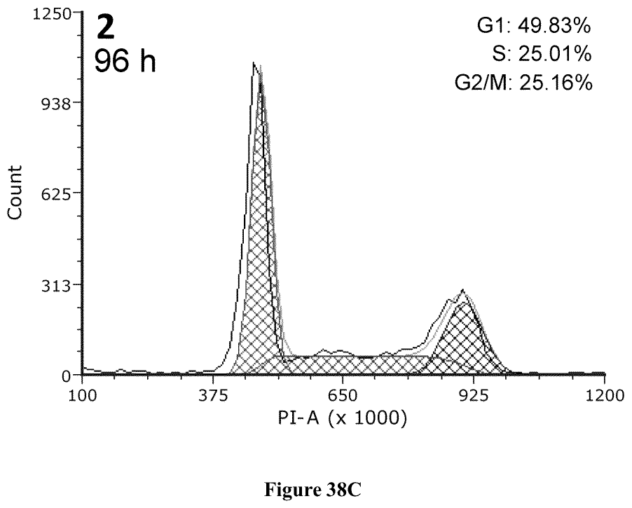

Persistent unrepaired DNA damage mostly results in cell cycle arrest and apoptosis. Therefore, the inventors investigated the effect of cyclic peptide 2 on the cycle progression of HeLa cells that were treated with 1 μM of cyclic peptide 2 for 72 and 96 h and subjected to flow cytometric analysis. They observed that cyclic peptide 2 treatment led to an increase in the population of cells at G2/M phase after 72 h (˜5.2% and ˜13% after 72 and 96 h, respectively), (FIGS. 4J and 38). Subsequently, they examined the rate of apoptosis in HeLa cells, treated with cyclic peptide 2 using Annexin V-FITC/PI double staining kit. The inventor's results show that treating HeLa cells with 1 μM of cyclic peptide 2 for 96 h led to an increase of overall Annexin V positive cells (˜14.5%) (FIGS. 4K and 37). These results suggest that cyclic peptide 2 treatment leads to elevated apoptosis. Altogether, the inventors provide evidence that cyclic peptide 2 inhibits DSB repair through binding to Lys63-linked Ub chains which leads to G2/M cell cycle arrest and apoptosis.

### Example 4

### Identification of Possible Ligands for Cyclic Peptides Targeting K63 Ub

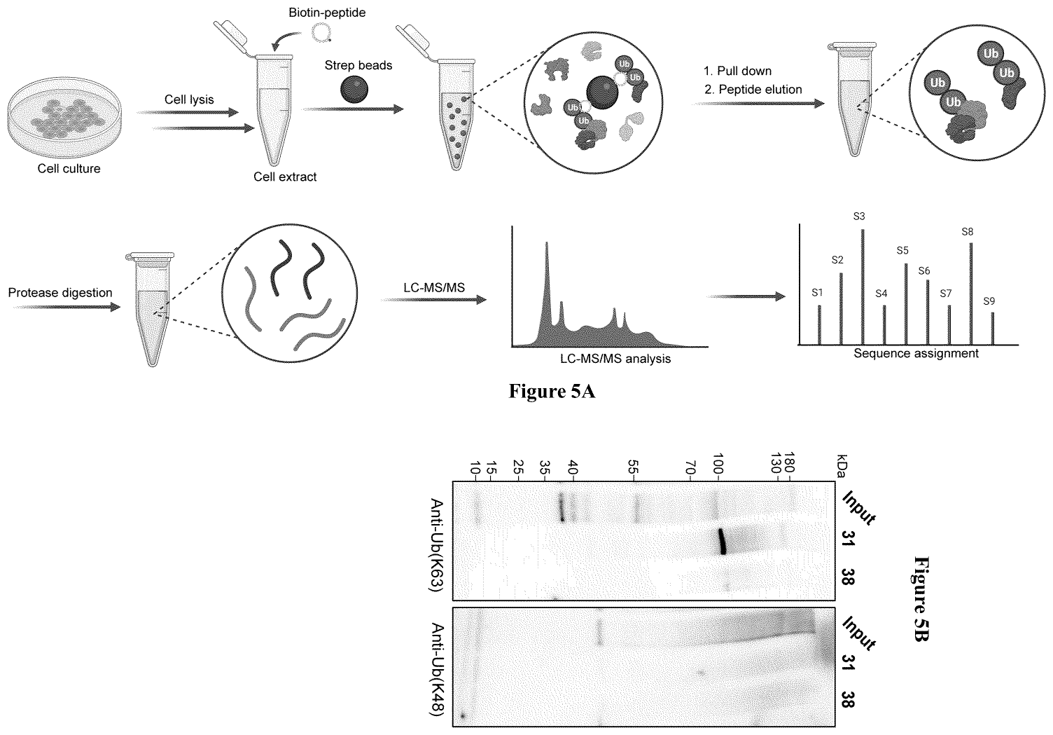

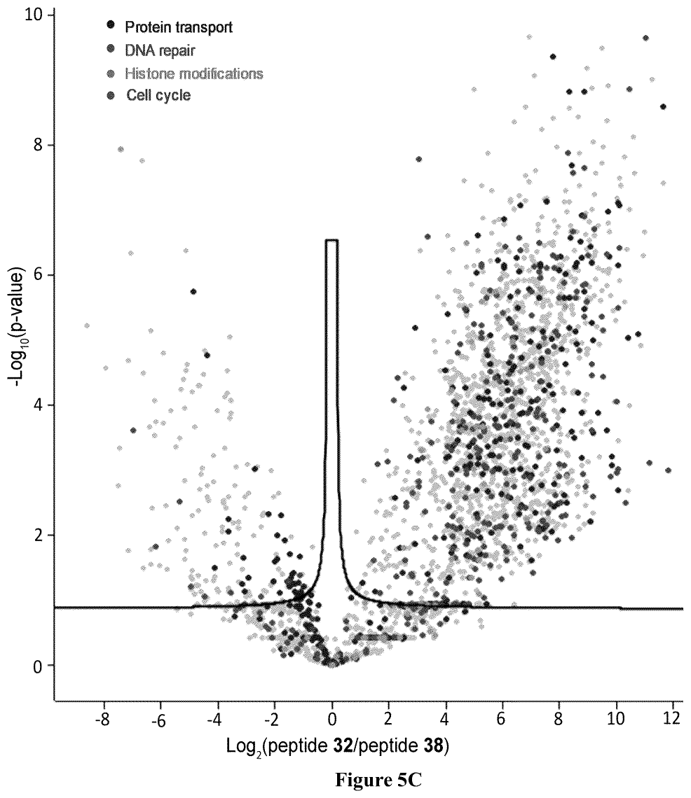

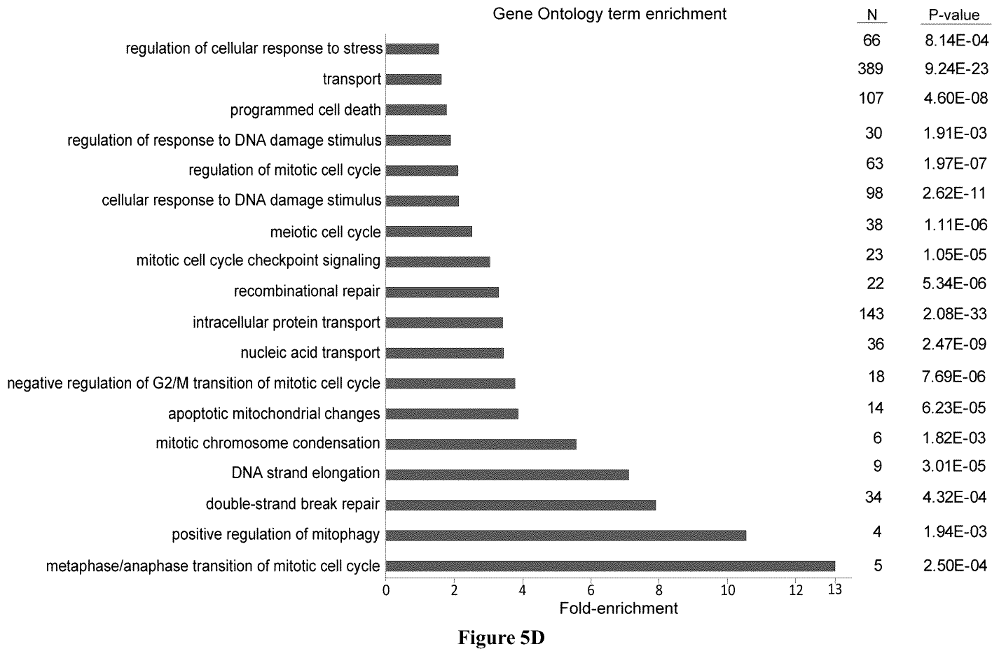

To explore which ubiquitinated proteins modified via Lys63-linked Ub chains could bind to cyclic peptide 2, the inventors modified its structure with an S-biotinylation reagent, generating 31, for the pulldown experiment. They utilized derivative 31 and streptavidin beads to enrich Lys63-linked Ub chains and their binders from U2OS lysate, including the biotinylated scrambled peptide 38 as a nonspecific control. These binders were separated by sodium dodecyl sulfate-polyacrylamide gel electrophoresis (SDS-PAGE), followed by western blot analysis with antibodies for Lys63-linked and Lys48-linked Ub chains (FIG. 5B). Cyclic peptide 31 specifically enriched proteins modified with Lys63-linked Ub chains without Lys48-linked chains, which supports the specificity of Lys63-linked Ub chains binding by cyclic peptide 2.

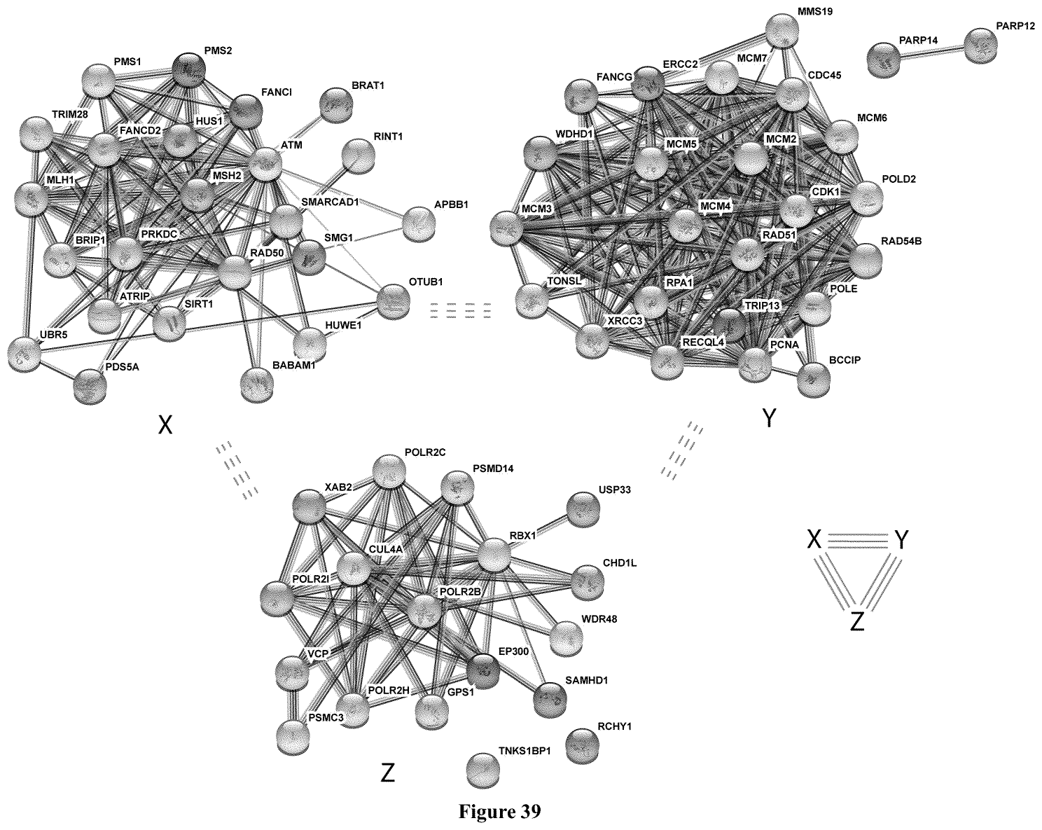

To identify the proteins enriched by 31, the inventors performed on-bead digestion followed by label-free proteomics (FIG. 5A). The inventors enriched a significant amount of proteins (˜1,100), in which a substantial number of proteins (˜450) are involved various cellular processes where Lys63-linked Ub chains are primarily involved (such as DNA repair, transport, cell cycle, and histone modifications), shown by the color dots in the volcano plot (FIG. 5C). The inventors have identified 68 improved terms of DNA repair proteins enriched by 31, showed in a cluster form with string networks (FIG. 39, and Table 2). Importantly, the inventors have enriched a few proteins that has specific affinity to K63-Ub chains, such as UBR5, PCNA, BABAM1, and PSMD14. The gene ontology analysis showed enriched G0 terms for DSB repair, regulation of mitotic cell cycle, mitotic chromosome condensation, transport, and response to stress, and others (FIG. 5D), suggesting that 31 exclusively pull-down proteins attached to K63-linked ubiquitin. All these results together imply that cyclic peptide 2 specifically binds to Lys63-linked Ub chains, hence regulates cellular processes like DDR, cell cycle, etc. in which Lys63 chain type is predominantly involved.

### CONCLUSIONS

Current therapeutic approaches to target components of the ubiquitination machinery focus on inhibiting the activity of a specific enzyme. Modulating DNA damage repair via targeting Lys63-linked Ub chains is a novel approach that has never been tested before. Furthermore, the absence of pharmacological inhibitors of Lys63-linked Ub chains E2-E3 enzymes highlight the urgency for establishing orthogonal approaches for targeting Lys63-linked Ub chains. As the vast majority of human cancers have a defective repair pathway, targeting the remaining functional repair pathways by modulating Lys63-linked Ub chains has great therapeutic potential. While many therapeutic approaches suffer from acquired drug resistance, the inventors believe that targeting the conserved Lys63-linked Ub chains might be able to escape the canonical drug resistance mechanisms. Furthermore, developing cyclic peptides for targeting Lys63-linked Ub chains increases the repertoire of druggable therapeutic targets.

The inventors discovered macrocyclic modulators of Lys63-linked Di-Ub by combining chemical protein synthesis, RaPID selection, and late-stage modifications. They exploited the power of chemical synthesis to produce an additional library of Cys mutants and their modified analogs. This multidisciplinary screening approach produced efficient cyclic peptides binders that distinguish between different Ub chain types and tightly bind to Lys63 chains. This discovery is remarkable considering the flexible and opened structure of the Lys63 chain in solution. Importantly, the effective cyclic peptide does not bind to the linear Di-Ub chain, which has a similar structural feature to that of Lys63-linked Di-Ub. The molecular basis for such selectivity and the mode of interactions of the herein disclosed cyclic peptides with the Lys63-linked chain remain to be determined, which would further enable their modifications for improving their functional properties. The inventors observed that a slight structural modification in cyclic peptide-based Ub-binders extensively affects the binding efficiency. This highlights the strength of the current approach in selecting specific Ub-chain binders, despite subtle differences at the molecular level, for potential drug development.

The inventors discovered cyclic peptide is a cell-permeable modulator of the DDR pathway which leads to DNA damage accumulation, cell cycle arrest in G2/M phases, and apoptosis. Moreover, proteomic analysis of proteins that were enriched with their cyclic peptide revealed crucial elements of the DDR pathway involving the Lys63-linked Ub chain.

The inventors approach provides new opportunities in basic research associated with the Ub system. They envision that this macrocyclic peptide will become a valuable tool to modulate Ub signaling and DNA damage. The selective inhibition of Lys63-linked polyubiquitin chains by cyclic peptides could be a promising strategy for cancer therapy.

### Example 5

### Optimization of Cyclic Peptide Solubilization and Cell Entry Capability

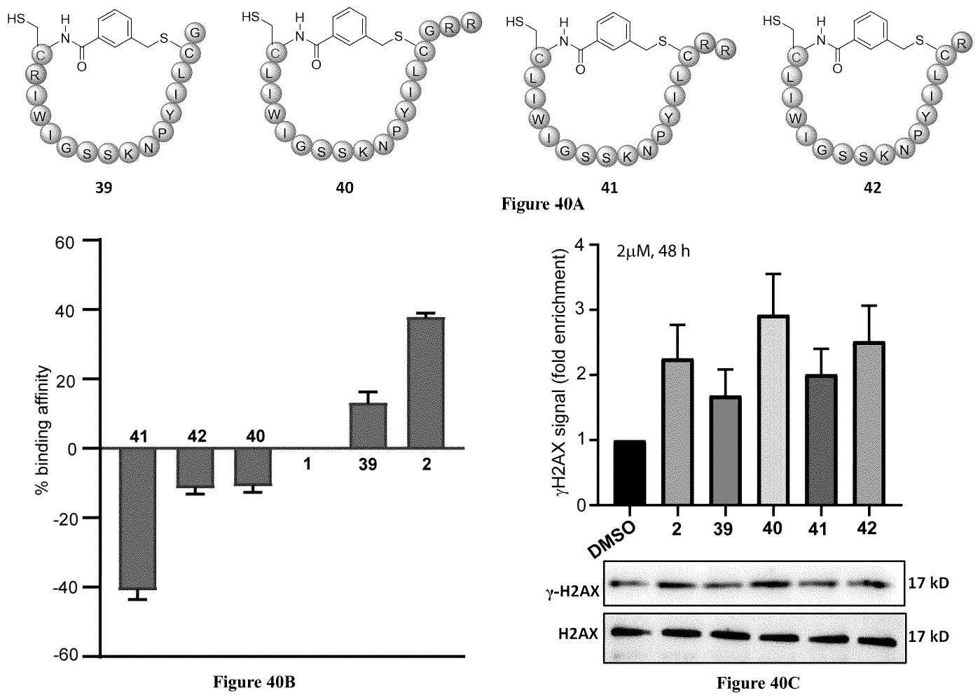

The current cyclic peptide modulator of Lys63-linked Di-Ub by combining chemical protein synthesis, RaPID selection, and late-stage modifications, 2 (CP2), has poor aqueous solubility, which limits its applicability in advance biological studies and other applications. To overcome this challenge, the inventors incorporated Arg (R) at the endo and exocyclic position to generate 39-42 (FIG. 40A), which improved the overall solubility of the cyclic peptide (5- to 10-fold more than 2) in the buffer (aq.) medium. Interestingly, the disruption of DNA damage repair ability of the cyclic peptide with ‘RR’ modification at the exocyclic position (40) was relatively higher compared to the parent cyclic peptide 2, even if, it showed relatively low binding affinity towards Lys63-linked Di-Ub than 2 (FIGS. 40B-40C). This could be reasoned that the modification with RR improved the cellular permeability of the cyclic peptide. Therefore, the inventors concluded that the cellular entry of the cyclic peptide can be further improved by the structural tuning to the cyclic peptide at other position(s).

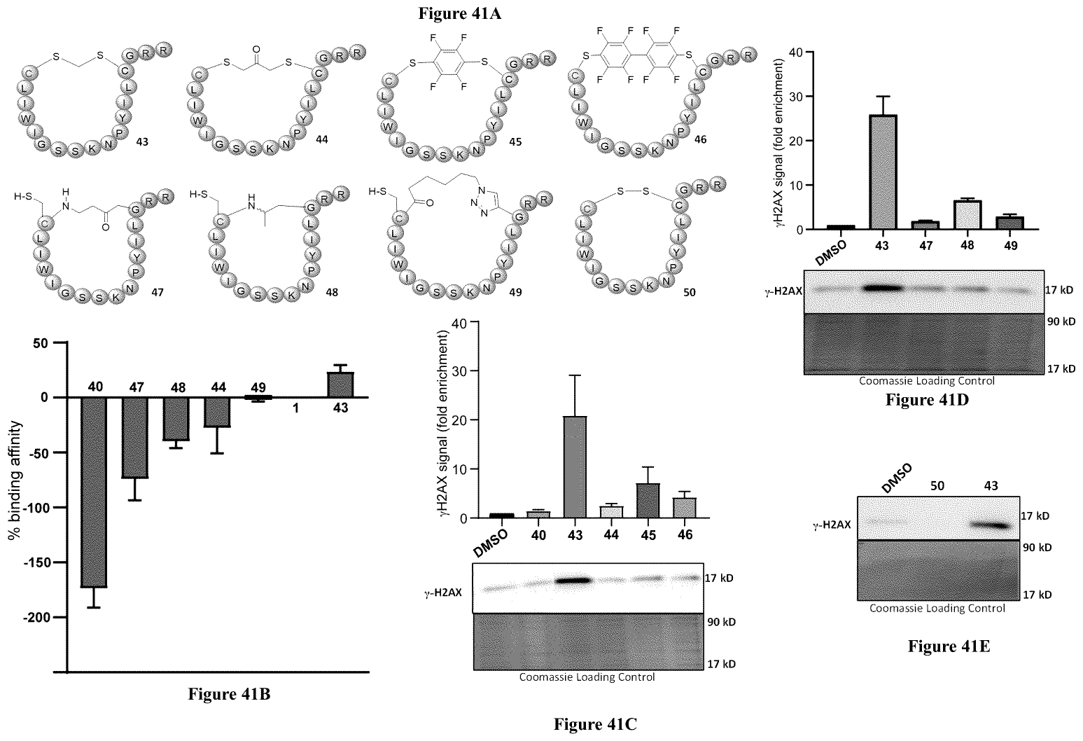

The inventors hypothesized that the linker for cyclization is an important factor to provide unique conformational properties to the cyclic peptide. To investigate this, the inventors have prepared eight different linkages on the RR peptide to generate a library, 43-50 (FIG. 41A). The current in vitro binding affinity and in-cell DNA damage induction screening results showed that 43 has the uppermost binding affinity as well as significantly improved DNA damage induction ability among all the cyclic peptides (FIGS. 41B-41C). This implies that the ‘linker of cyclization’ plays a key role in improving cell entry of the cyclic peptides.

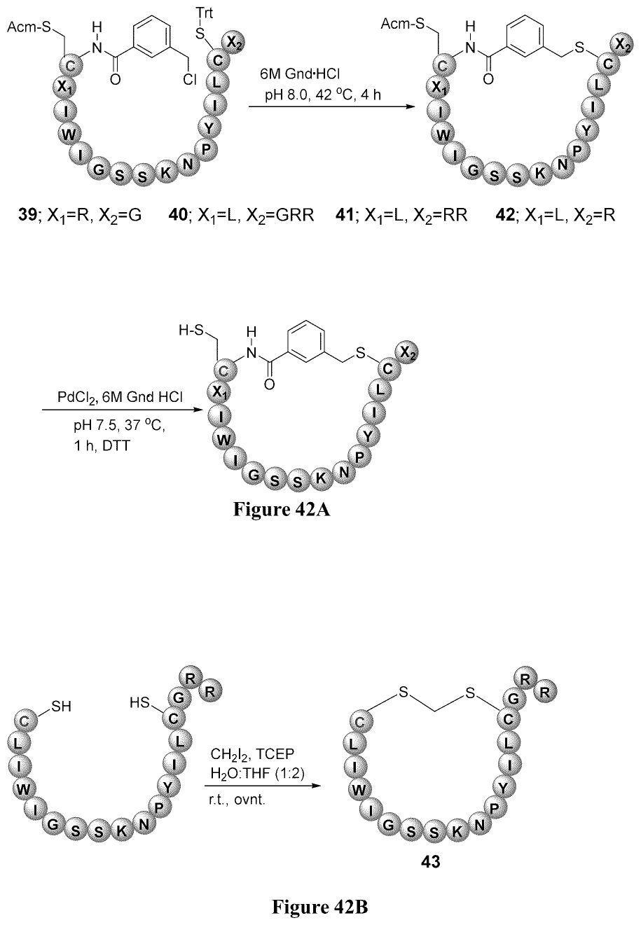

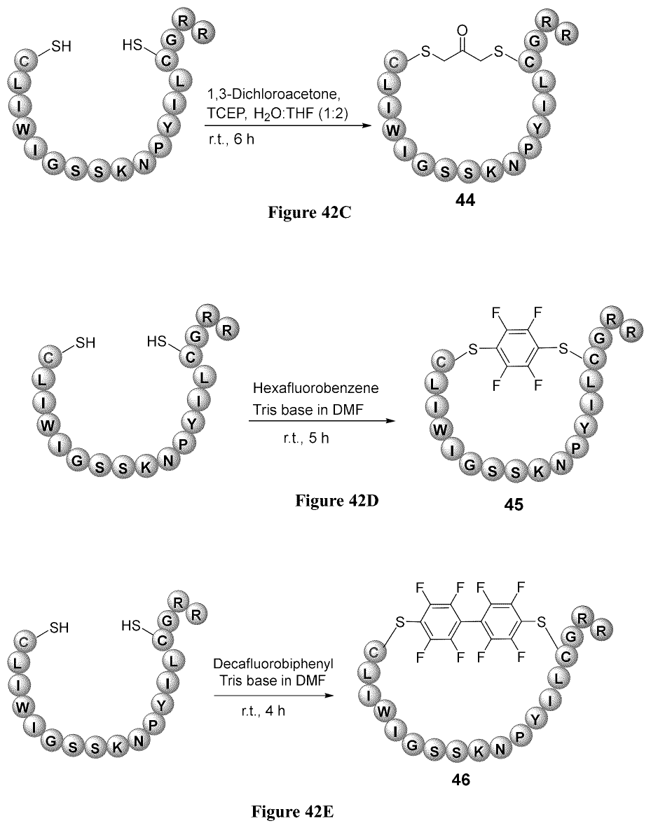

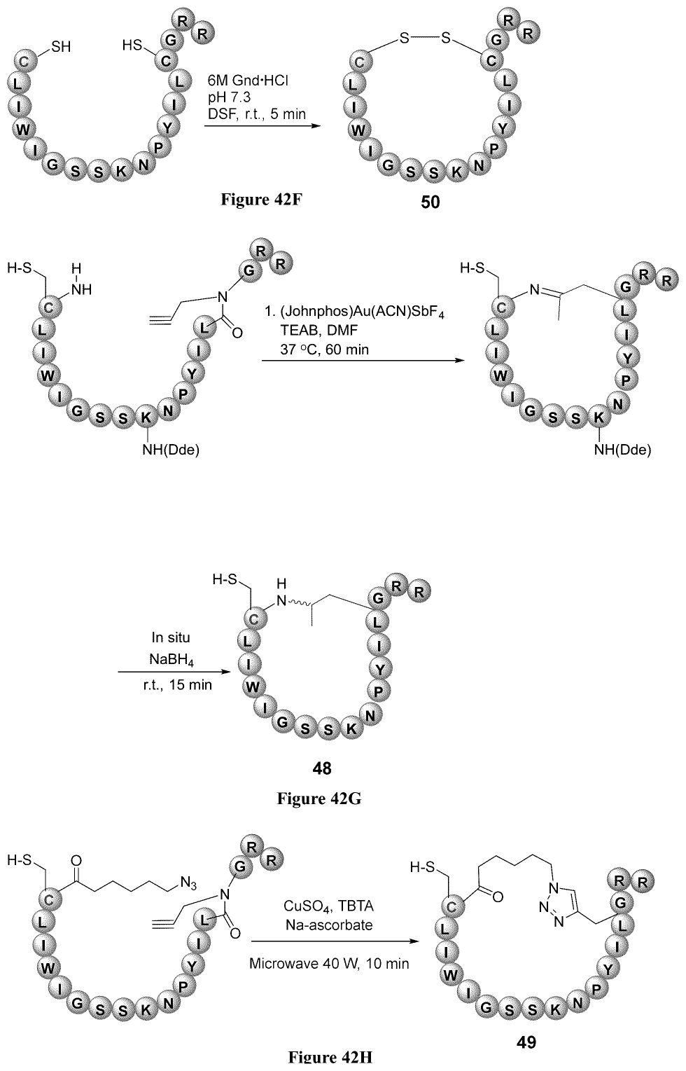

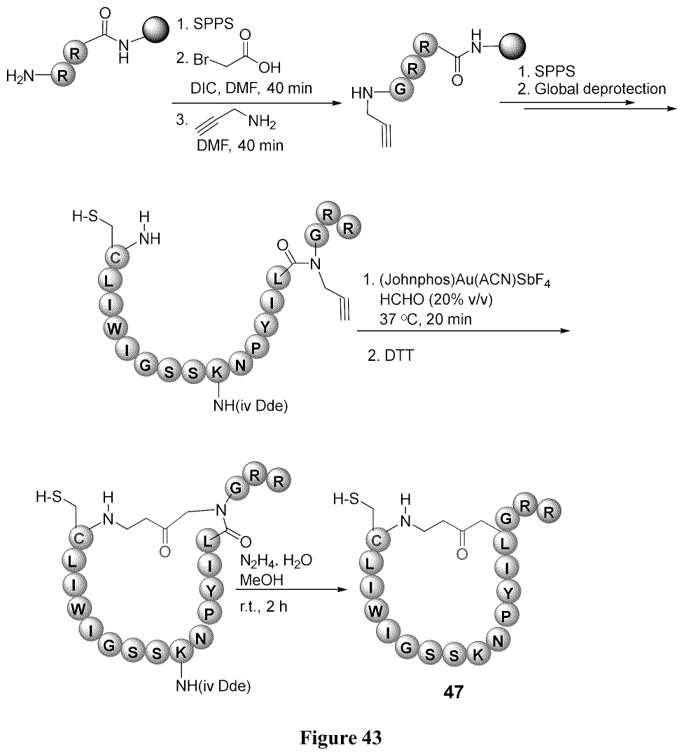

Non-limiting exemplary synthetic schemes of the peptides 39-50 are presented in FIGS. 42-43.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A cyclic peptide comprising the amino acid sequence: LLIWIGSSKNPYILCG (SEQ ID NO: 1) or a functional analog thereof having at least 80% homology thereto.

2. The cyclic peptide of claim 1, comprising at least one cysteine residue substituting an amino acid residue of said SEQ ID NO: 1.

3. The cyclic peptide of claim 1, comprising an amino acid sequence selected from the group consisting of: CLIWIGSSKNPYILCG (SEQ ID NO: 2); LCIWIGSSKNPYILCG (SEQ ID NO: 3); LLCWIGSSKNPYILCG (SEQ ID NO: 4) LLICIGSSKNPYILCG (SEQ ID NO: 5); LLIWCGSSKNPYILCG (SEQ ID NO: 6); LLIWICSSKNPYILCG (SEQ ID NO: 7) LLIWIGCSKNPYILCG (SEQ ID NO: 8); LLIWIGSCKNPYILCG (SEQ ID NO: 9); LLIWIGSSKCPYILCG (SEQ ID NO: 10); LLIWIGSSKNCYILCG (SEQ ID NO: 11); LLIWIGSSKNPCILCG (SEQ ID NO: 12); and LLIWIGSSKNPYCLCG (SEQ ID NO: 13).

4. The cyclic peptide of claim 1, comprising 14 to 20 amino acid residues.

5. The cyclic peptide of claim 1, wherein the amino acid at position one of the N terminus is conjugated to a cyclizing molecule.

6. The cyclic peptide of claim 5, wherein said cyclizing molecule comprises any one of: 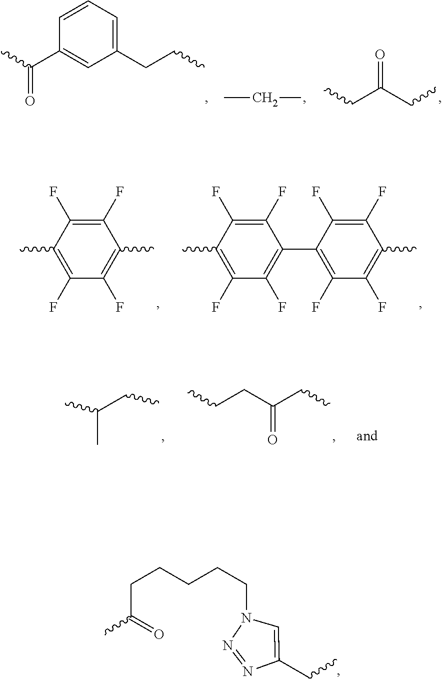
wherein each wavy bond represents an attachment point to the first amino acid residue or to the C-terminal amino acid, respectively.

7. The cyclic peptide of claim 1, wherein said cyclizing molecule is —CH₂—.

8. The cyclic peptide of claim 1, wherein said cyclic peptide is chemically modified, and optionally wherein said chemical modification is selected from the group consisting of: alkylation, arylation, addition of a thiol protecting group, and any combination thereof.

9. (canceled)

10. The cyclic peptide claim 1, being characterized by having: cell penetration capability, ubiquitin (Ub) binding capability, or any combination thereof, optionally wherein said Ub is a polymeric Ub, and optionally wherein said polymeric Ub comprises Ub monomers linked at their Lysine at position 63 (K63Ub).

11.-12. (canceled)

13. The cyclic peptide of claim 1, having increased affinity to Lys63-linked Ub chain, compared to a control Ub chain, and optionally wherein said increased affinity is binding affinity with a dissociation constant (KD) of 0.05-150 nM.

14. (canceled)

15. The cyclic peptide of claim 1, further comprising at least one arginine reside.

16. The cyclic peptide of claim 15, wherein said at least one arginine reside is located at the C-terminus of said cyclic peptide.

17. The cyclic peptide of claim 15, comprising an amino acid sequence selected from the group consisting of: CLIWIGSSKNPYILCGRR (SEQ ID NO: 15); CLIWIGSSKNPYILCRR (SEQ ID NO: 16); and CLIWIGSSKNPYILCR (SEQ ID NO: 17).

18. A dimeric cyclic peptide comprising the cyclic peptide of claim 1.

19. A pharmaceutical composition comprising the cyclic peptide of claim 1 and an acceptable carrier.

20.-22. (canceled)

23. A method for ameliorating or treating a K63Ub-related disease in a subject in need thereof, the method comprising administering to the subject a therapeutically effective amount of the cyclic peptide of claim 1,
thereby ameliorating or treating a K63Ub related disease in the subject.

24. The method of claim 23, wherein said K63Ub-related disease is a cell proliferation-related disease.

25. The method of claim 24, wherein said cell-proliferation related disease is cancer.

26. The method of claim 23, wherein said ameliorating or treating comprises: increasing an amount of fragmented DNA in a cell of said subject, increasing an amount, rate, or both, of cell apoptosis in said subject, or any combination thereof, and optionally wherein said cell is a caner or cancerous cell.

27. (canceled)

28. The method of claim 23, further comprising administering to said subject a therapeutically effective amount of an anticancer agent.


SEQUENCE LISTING (17 sequences)

SEQ ID NO: 1 - protein, 16 aa, synthetic construct
LLIWIGSSKNPYILCG

SEQ ID NO: 2 - protein, 16 aa, synthetic construct
CLIWIGSSKNPYILCG

SEQ ID NO: 3 - protein, 16 aa, synthetic construct
LCIWIGSSKNPYILCG

SEQ ID NO: 4 - protein, 16 aa, synthetic construct
LLCWIGSSKNPYILCG

SEQ ID NO: 5 - protein, 16 aa, synthetic construct
LLICIGSSKNPYILCG

SEQ ID NO: 6 - protein, 16 aa, synthetic construct
LLIWCGSSKNPYILCG

SEQ ID NO: 7 - protein, 16 aa, synthetic construct
LLIWICSSKNPYILCG

SEQ ID NO: 8 - protein, 16 aa, synthetic construct
LLIWIGCSKNPYILCG

SEQ ID NO: 9 - protein, 16 aa, synthetic construct
LLIWIGSCKNPYILCG

SEQ ID NO: 10 - protein, 16 aa, synthetic construct
LLIWIGSSKCPYILCG

SEQ ID NO: 11 - protein, 16 aa, synthetic construct
LLIWIGSSKNCYILCG

SEQ ID NO: 12 - protein, 16 aa, synthetic construct
LLIWIGSSKNPCILCG

SEQ ID NO: 13 - protein, 16 aa, synthetic construct
LLIWIGSSKNPYCLCG

SEQ ID NO: 14 - protein, 16 aa, synthetic construct
CRIWIGSSKNPYILCG

SEQ ID NO: 15 - protein, 18 aa, synthetic construct
CLIWIGSSKNPYILCGRR

SEQ ID NO: 16 - protein, 17 aa, synthetic construct
CLIWIGSSKNPYILCRR

SEQ ID NO: 17 - protein, 16 aa, synthetic construct
CLIWIGSSKNPYILCR